SEQUENCE LISTING (23 sequences)

SEQ ID NO: 1 - DNA, 6368 bp, Zea mays
  source 1..6368: mol_type = genomic DNA
ggagcttctcctcttagaacgtcgatgaacagtgtagacttattcaatacattaatgtcattttgagatccggcagtaccaaaaaaagcatgccatatgcgaagatctcgtgatgcaactgcttcaagaatcatagtaggaacgcctttgtcaccacgtgtaaacatgccggcccaaccttttgggcaattcttccaagcccaatgcatacagtcaatgcttcctatcatccctggaaaaccacgagactcattttgttgaagtatattttctagctcgtcactccttggaggacgcaagtactctggaccaaagcattctatcacccctttggcaaaatttcccaaacactctaaagccgtgcttgtgccaatcttcaaaacttcatcaagttgatcagctgagctgccatatgcaagcatccgaattgccgcagtacacttttggagtggtgagtggccatctttaccagtgacatctgctcgctgtgtaaaataagaggaccactcaccaagtttttccacaatgcgaagaaaaagcggccttctcatacggaacctcctacggaacatctcgtcggtgtagataggatttgcagagaaataatttgctacaagatcatcatgcgcaccttgtcgattccttggaataactctcctgattccggagaggcgtctgcggcgaggtggattcgatgcttctccatcgattgcaaccttcaaattctcttcgatcttccttccaaattcatctaatatttcatcctcgacaaggaaatcttctaacgtgtaaacttcactgggatccaactcgaaaggatcaaactctccctctggttcgtttgacattgtggatggagtgactaacctgctaacaccctgcaacaatttatacaggagcatatcctcatgcacacgcaaaactgatgttgtccacaagacacgcacaggacacgcaaacagtttcagactcatgcacacgcacatcagtttcagactcaggcacacgcacatcaaatcaccttcgcttgtcgatgagtcgcagccgcatcgtacaatggcgattttaccgacgataaggcatgggagcacgagccgtcgtcgtcgccttgcgagacgacgggagcgatctctcccttcatttaatctcttccacgtcaggttattttgctgagatggcagtatacagacggcaaagttaatgccgttgtacatgcccttagactcttccgtcaccaactcacttagatttttacaacggaacataaggttcgcttgcagacttacatataaggtatagttgcataataatcgccttatgctgtacattgcgacacccgtaaatattcgatgaaatattagtacacaatattaaataagaacgaacaatacatatattatcattgatcttagtatctccttttgctcctcgtagaacaattctgtgtaaattatgcgtaaaattcgaggaccaaaacattggctagaaaaatacctaaaatcagttttgcaattgtttctgattttcctcatattttcttgcttataaagttttccaaaagtaccattttggatgaaaaaacggaaaacaacgctggtctacttgtaaatttggtagtgacatttgggaccgtctagacacgacctaaaaatagtagtctaaaacatagtctgacacgatgccttaaaaatagacgacaaagcacaacacgattagatgtgtcgtgttttgaccgacacgacacaaagtaaggcacgatttaaaacccaataaataatattttaatggttattttatgttccaataattttcatctcttcaaaaaaatgttatagaaatcattgatacttagttgaatatcctaacacaatatatatatatattaatatatatatatcaattttaagtcactttgctagacatagtaatatattttaaatattttctctttcttgtatatttttaaaatacacatcagtttttatatgtgtcgtgcttgaaccgacacgatataatcatcggttcgccgtacttctagatcatgatgttcctaggttttaatattaagagacggtctatattaactcaaaactatttcgtgaaaggctaactcgaaaaaaaaatgaatgtaatcacggcccgtcctggattcgagattctaacgtttcattcgtgtccagtgtgcacacttgtggaaaaggaagacgaagaaaaaaaccaacaactaactccggcccgccggatgcgcccacctacttccccctcgcccctctcatggtctctctcgcgcccagatctgctactagacggcaccgctgcagcgcgtcgtgtcgcgggggttggtggcaggcagcgagagcttgccgttcctctctctcagttgtcaggtcctaggctcacctcaccggctcccagcccgcttctatttcttcctccccgaccccgtgcaggtggcagtccagtccacgccaccaaccgcgaggcgaaccaaaccaacccactctccccaaccccgcgcgcccaggccgcccgccctaccaaccatcggcgtcggcaatggcggccatggcgaccaaggccgccgcgggcaccgtgtcgctggacctcgccgcgccgtcgcgccgccaccaccgcccgagctcggcgcgcccgcccgcccgccccgccgtccgcgggctgcgggcgcctgggcgccgcgtgatcgccgcgccgccggcggcggcagcggcggcggcggtgcaggcgggtgccgaggagatcgtgctgcagcccatcaaggagatctccggcaccgtcaagctgccggggtccaagtcgctttccaaccggatcctcctgctcgccgccctgtccgaggtgagcgattttggtgcttgctgcgctgccctgtctcactgctacctaaatgttttgcctgtcgaataccatggattctcggtgtaatccatctcacgatcagatgcaccgcatgtcgcatgcctagctctctctaatttgtctagtagtttgtatacggattaatattgataaatcggtaccgcaaaagctaggtgtaaataaacactagaaaattggatgttcccctatcggcctgtactcggctactcgttcttgtgatggcatgctgtctcttcttggtgtttggtgaacaaccttatgaaatttgggcgcaaagaactcgccctcaagggttgatcttatgccatcgtcatgataaacagtggagcacggacgatcctttacgttgtttttaacaaactttgtcagaaaactagcatcattaacttcttaatgacgatttcacaacaaaaaaaggtaacctcgctactaacataacaaaatacttgttgcttattaattatatgttttttaatctttgatcaggggacaacagtggttgataacctgttgaacagtgaggatgtccactacatgctcggggccttgaggactcttggtctctctgtcgaagcggacaaagctgccaaaagagctgtagttgttggctgtggtggaaagttcccagttgaggattctaaagaggaagtgcagctcttcttggggaatgctggaactgcaatgcggccattgacagcagctgttactgctgctggtggaaatgcaacgtatgtttcctctctttctctctacaatacttgctggagttagtatgaaacccatgggtatgtctagtggcttatggtgtattggtttttgaacttcagttacgtgcttgatggagtaccaagaatgagggagagacccattggcgacttggttgtcggattgaagcagcttggtgcagatgttgattgtttccttggcactgactgcccacctgttcgtgtcaatggaatcggagggctacctggtggcaaggttagctactaagggccacatgttacattcttctgtaaatggtacaactattgtcgagcttttgcatttgtaaggaaagcattgattgatctgaatttgatgctacaccacaaaatatcctacaaatggtcatccctaactagcaaacaatgaagtaatacttggcatgtgtttatcaaattaatttccatcttctggggcattgcctgttttctagtctaatagcatttgtttttagcattaattagctcttacaattgttatgttctacaggtcaagctgtctggctccatcagcagtcagtacttgagtgccttgctgatggctgctcctttggctcttggggatgtggagattgaaatcattgataaattaatctccattccctacgtcgaaatgacattgagattgatggagcgttttggtgtgaaagcagagcattctgatagctgggacagattctacattaagggaggtcaaaaatacaagtaagctctgtaatgtatttcactactttgatgccaatgtttcagttttcagttttccaaacagtcgcatcaatatttgaatagatgcactgtagaaaaaaaatcattgcagggaaaaactagtactgagtattttgactgtaaattattttaccagtcggaatatagtcagtctattggagtcaagagcgtgaaccgaaatagccagttaattatcccattatacagaggacaaccatgtatactattgaaacttggtttataagagaatctaggtagctggactcgtagctgcttggcatggataccttcttatctttaggaaaagacacttgatttttttttttctgtggccctctatgatgtgtgaacctgcttctctattgctttagaaggatatatctatgtcgttatgcaacatgcttcccttagccatttgtactgaaatcagtttcataagttcgttagtggttccctaaacgaaaccttgtttttctttgcaatcaacaggtcccctaaaaatgcctatgttgaaggtgatgcctcaagcgcaagctatttcttggctggtgctgcaattactggagggactgtgactgtggaaggttgtggcaccaccagtttgcaggtaaagatttcttggctggtgctacaataactgcttttgtctttttggtttcagcattgttctcagagtcactaaataacattatcatctgcaaatgtcaaatagacatacttaggtgaattcatgtaaccgtttccttacaaatttgctgaaacctcagggtgatgtgaagtttgctgaggtactggagatgatgggagcgaaggttacatggaccgagactagcgtaactgttactggcccaccgcgggagccatttgggaggaaacacctcaaggcgattgatgtcaacatgaacaagatgcctgatgtcgccatgactcttgctgtggttgccctctttgccgatggcccgacagccatcagagacggtaaaacattctcagccctacaaccatgcctcttctacatcactacttgacaagactaaaaactattggctcgttggcagtggcttcctggagagtaaaggagaccgagaggatggttgcgatccggacggagctaaccaaggtaaggctacatacttcacatgtctcacgtcgtctttccatagctcgctgcctcttagcggcttgcctgcggtcgctccatcctcggttgctgtctgtgttttccacagctgggagcatctgttgaggaagggccggactactgcatcatcacgccgccggagaagctgaacgtgacggcgatcgacacgtacgacgaccacaggatggccatggccttctcccttgccgcctgtgccgaggtccccgtggccatccgggaccctgggtgcacccggaagaccttccccgactacttcgatgtgctgagcactttcgtcaagaattaataaagcgtgcgatactaccacgcagcttgattgaagtgataggcttgtgctgaggaagtacatttcttttgttctgttttttctctttcacgggattaagttttgagtctgtaacgttagttgtttgtagcaagtttctatttcggatcttaagtttgtgcactgcaagccaaatttcatttcaagagtggttcgttggaataataagaataataaattacgtttcagtggctgtcaagcctgctgctacgttttaggagatggcattagacattcatcatcaacaacaataaaaccttttagcctcaaacaataatagtgaagttattttttagtcctaaacaagttgcattaggatatagttaaaacacaaaagaagctaaagttagggtttagacatgtggatattgttttccatgtgtagtatgttctttctttgagtctcatttaactacctctacacataccaactttagttttttttctacctcttcatgttactatggtgccttcttatcccactgagcattgg

SEQ ID NO: 2 - DNA, 3280 bp, Zea mays
  source 1..3280: mol_type = other DNA
atggcggccatggcgaccaaggccgccgcgggcaccgtgtcgctggacctcgccgcgccgtcgcgccgccaccaccgcccgagctcggcgcgcccgcccgcccgccccgccgtccgcgggctgcgggcgcctgggcgccgcgtgatcgccgcgccgccggcggcggcagcggcggcggcggtgcaggcgggtgccgaggagatcgtgctgcagcccatcaaggagatctccggcaccgtcaagctgccggggtccaagtcgctttccaaccggatcctcctgctcgccgccctgtccgaggtgagcgattttggtgcttgctgcgctgccctgtctcactgctacctaaatgttttgcctgtcgaataccatggattctcggtgtaatccatctcacgatcagatgcaccgcatgtcgcatgcctagctctctctaatttgtctagtagtttgtatacggattaatattgataaatcggtaccgcaaaagctaggtgtaaataaacactagaaaattggatgttcccctatcggcctgtactcggctactcgttcttgtgatggcatgctgtctcttcttggtgtttggtgaacaaccttatgaaatttgggcgcaaagaactcgccctcaagggttgatcttatgccatcgtcatgataaacagtggagcacggacgatcctttacgttgtttttaacaaactttgtcagaaaactagcatcattaacttcttaatgacgatttcacaacaaaaaaaggtaacctcgctactaacataacaaaatacttgttgcttattaattatatgttttttaatctttgatcaggggacaacagtggttgataacctgttgaacagtgaggatgtccactacatgctcggggccttgaggactcttggtctctctgtcgaagcggacaaagctgccaaaagagctgtagttgttggctgtggtggaaagttcccagttgaggattctaaagaggaagtgcagctcttcttggggaatgctggaactgcaatgcggccattgacagcagctgttactgctgctggtggaaatgcaacgtatgtttcctctctttctctctacaatacttgctggagttagtatgaaacccatgggtatgtctagtggcttatggtgtattggtttttgaacttcagttacgtgcttgatggagtaccaagaatgagggagagacccattggcgacttggttgtcggattgaagcagcttggtgcagatgttgattgtttccttggcactgactgcccacctgttcgtgtcaatggaatcggagggctacctggtggcaaggttagctactaagggccacatgttacattcttctgtaaatggtacaactattgtcgagcttttgcatttgtaaggaaagcattgattgatctgaatttgatgctacaccacaaaatatcctacaaatggtcatccctaactagcaaacaatgaagtaatacttggcatgtgtttatcaaattaatttccatcttctggggcattgcctgttttctagtctaatagcatttgtttttagcattaattagctcttacaattgttatgttctacaggtcaagctgtctggctccatcagcagtcagtacttgagtgccttgctgatggctgctcctttggctcttggggatgtggagattgaaatcattgataaattaatctccattccctacgtcgaaatgacattgagattgatggagcgttttggtgtgaaagcagagcattctgatagctgggacagattctacattaagggaggtcaaaaatacaagtaagctctgtaatgtatttcactactttgatgccaatgtttcagttttcagttttccaaacagtcgcatcaatatttgaatagatgcactgtagaaaaaaaatcattgcagggaaaaactagtactgagtattttgactgtaaattattttaccagtcggaatatagtcagtctattggagtcaagagcgtgaaccgaaatagccagttaattatcccattatacagaggacaaccatgtatactattgaaacttggtttataagagaatctaggtagctggactcgtagctgcttggcatggataccttcttatctttaggaaaagacacttgatttttttttttctgtggccctctatgatgtgtgaacctgcttctctattgctttagaaggatatatctatgtcgttatgcaacatgcttcccttagccatttgtactgaaatcagtttcataagttcgttagtggttccctaaacgaaaccttgtttttctttgcaatcaacaggtcccctaaaaatgcctatgttgaaggtgatgcctcaagcgcaagctatttcttggctggtgctgcaattactggagggactgtgactgtggaaggttgtggcaccaccagtttgcaggtaaagatttcttggctggtgctacaataactgcttttgtctttttggtttcagcattgttctcagagtcactaaataacattatcatctgcaaatgtcaaatagacatacttaggtgaattcatgtaaccgtttccttacaaatttgctgaaacctcagggtgatgtgaagtttgctgaggtactggagatgatgggagcgaaggttacatggaccgagactagcgtaactgttactggcccaccgcgggagccatttgggaggaaacacctcaaggcgattgatgtcaacatgaacaagatgcctgatgtcgccatgactcttgctgtggttgccctctttgccgatggcccgacagccatcagagacggtaaaacattctcagccctacaaccatgcctcttctacatcactacttgacaagactaaaaactattggctcgttggcagtggcttcctggagagtaaaggagaccgagaggatggttgcgatccggacggagctaaccaaggtaaggctacatacttcacatgtctcacgtcgtctttccatagctcgctgcctcttagcggcttgcctgcggtcgctccatcctcggttgctgtctgtgttttccacagctgggagcatctgttgaggaagggccggactactgcatcatcacgccgccggagaagctgaacgtgacggcgatcgacacgtacgacgaccacaggatggccatggccttctcccttgccgcctgtgccgaggtccccgtggccatccgggaccctgggtgcacccggaagaccttccccgactacttcgatgtgctgagcactttcgtcaagaattaa

SEQ ID NO: 3 - DNA, 1521 bp, Zea mays
  source 1..1521: mol_type = other DNA
atggcggccatggcgaccaaggccgccgcgggcaccgtgtcgctggacctcgccgcgccgtcgcgccgccaccaccgcccgagctcggcgcgcccgcccgcccgccccgccgtccgcgggctgcgggcgcctgggcgccgcgtgatcgccgcgccgccggcggcggcagcggcggcggcggtgcaggcgggtgccgaggagatcgtgctgcagcccatcaaggagatctccggcaccgtcaagctgccggggtccaagtcgctttccaaccggatcctcctgctcgccgccctgtccgaggggacaacagtggttgataacctgttgaacagtgaggatgtccactacatgctcggggccttgaggactcttggtctctctgtcgaagcggacaaagctgccaaaagagctgtagttgttggctgtggtggaaagttcccagttgaggattctaaagaggaagtgcagctcttcttggggaatgctggaactgcaatgcggccattgacagcagctgttactgctgctggtggaaatgcaacttacgtgcttgatggagtaccaagaatgagggagagacccattggcgacttggttgtcggattgaagcagcttggtgcagatgttgattgtttccttggcactgactgcccacctgttcgtgtcaatggaatcggagggctacctggtggcaaggtcaagctgtctggctccatcagcagtcagtacttgagtgccttgctgatggctgctcctttggctcttggggatgtggagattgaaatcattgataaattaatctccattccctacgtcgaaatgacattgagattgatggagcgttttggtgtgaaagcagagcattctgatagctgggacagattctacattaagggaggtcaaaaatacaagtcccctaaaaatgcctatgttgaaggtgatgcctcaagcgcaagctatttcttggctggtgctgcaattactggagggactgtgactgtggaaggttgtggcaccaccagtttgcagggtgatgtgaagtttgctgaggtactggagatgatgggagcgaaggttacatggaccgagactagcgtaactgttactggcccaccgcgggagccatttgggaggaaacacctcaaggcgattgatgtcaacatgaacaagatgcctgatgtcgccatgactcttgctgtggttgccctctttgccgatggcccgacagccatcagagacgtggcttcctggagagtaaaggagaccgagaggatggttgcgatccggacggagctaaccaagctgggagcatctgttgaggaagggccggactactgcatcatcacgccgccggagaagctgaacgtgacggcgatcgacacgtacgacgaccacaggatggccatggccttctcccttgccgcctgtgccgaggtccccgtggccatccgggaccctgggtgcacccggaagaccttccccgactacttcgatgtgctgagcactttcgtcaagaattaa

SEQ ID NO: 4 - DNA, 1335 bp, Zea mays
  source 1..1335: mol_type = other DNA
gcgggtgccgaggagatcgtgctgcagcccatcaaggagatctccggcaccgtcaagctgccggggtccaagtcgctttccaaccggatcctcctgctcgccgccctgtccgaggggacaacagtggttgataacctgttgaacagtgaggatgtccactacatgctcggggccttgaggactcttggtctctctgtcgaagcggacaaagctgccaaaagagctgtagttgttggctgtggtggaaagttcccagttgaggattctaaagaggaagtgcagctcttcttggggaatgctggaactgcaatgcggccattgacagcagctgttactgctgctggtggaaatgcaacttacgtgcttgatggagtaccaagaatgagggagagacccattggcgacttggttgtcggattgaagcagcttggtgcagatgttgattgtttccttggcactgactgcccacctgttcgtgtcaatggaatcggagggctacctggtggcaaggtcaagctgtctggctccatcagcagtcagtacttgagtgccttgctgatggctgctcctttggctcttggggatgtggagattgaaatcattgataaattaatctccattccctacgtcgaaatgacattgagattgatggagcgttttggtgtgaaagcagagcattctgatagctgggacagattctacattaagggaggtcaaaaatacaagtcccctaaaaatgcctatgttgaaggtgatgcctcaagcgcaagctatttcttggctggtgctgcaattactggagggactgtgactgtggaaggttgtggcaccaccagtttgcagggtgatgtgaagtttgctgaggtactggagatgatgggagcgaaggttacatggaccgagactagcgtaactgttactggcccaccgcgggagccatttgggaggaaacacctcaaggcgattgatgtcaacatgaacaagatgcctgatgtcgccatgactcttgctgtggttgccctctttgccgatggcccgacagccatcagagacgtggcttcctggagagtaaaggagaccgagaggatggttgcgatccggacggagctaaccaagctgggagcatctgttgaggaagggccggactactgcatcatcacgccgccggagaagctgaacgtgacggcgatcgacacgtacgacgaccacaggatggccatggccttctcccttgccgcctgtgccgaggtccccgtggccatccgggaccctgggtgcacccggaagaccttccccgactacttcgatgtgctgagcactttcgtcaagaattaa

SEQ ID NO: 5 - protein, 506 aa, Zea mays
MAAMATKAAAGTVSLDLAAPSRRHHRPSSARPPARPAVRGLRAPGRRVIAAPPAAAAAAAVQAGAEEIVLQPIKEISGTVKLPGSKSLSNRILLLAALSEGTTVVDNLLNSEDVHYMLGALRTLGLSVEADKAAKRAVVVGCGGKFPVEDSKEEVQLFLGNAGTAMRPLTAAVTAAGGNATYVLDGVPRMRERPIGDLVVGLKQLGADVDCFLGTDCPPVRVNGIGGLPGGKVKLSGSISSQYLSALLMAAPLALGDVEIEIIDKLISIPYVEMTLRLMERFGVKAEHSDSWDRFYIKGGQKYKSPKNAYVEGDASSASYFLAGAAITGGTVTVEGCGTTSLQGDVKFAEVLEMMGAKVTWTETSVTVTGPPREPFGRKHLKAIDVNMNKMPDVAMTLAVVALFADGPTAIRDVASWRVKETERMVAIRTELTKLGASVEEGPDYCIITPPEKLNVTAIDTYDDHRMAMAFSLAACAEVPVAIRDPGCTRKTFPDYFDVLSTFVKN

SEQ ID NO: 6 - protein, 444 aa, Zea mays [sequence omitted]

SEQ ID NO: 7 - DNA, 1335 bp, Zea mays
  source 1..1335: mol_type = other DNA
gcgggtgccgaggagatcgtgctgcagcccatcaaggagatctccggcaccgtcaagctgccggggtccaagtcgctttccaaccggatcctcctgctcgccgccctgtccgaggggacaacagtggttgataacctgttgaacagtgaggatgtccactacatgctcggggccttgaggactcttggtctctctgtcgaagcggacaaagctgccaaaagagctgtagttgttggctgtggtggaaagttcccagttgaggattctaaagaggaagtgcagctcttcttggggaatgctggaattgcaatgcggtcattgacagcagctgttactgctgctggtggaaatgcaacttacgtgcttgatggagtaccaagaatgagggagagacccattggcgacttggttgtcggattgaagcagcttggtgcagatgttgattgtttccttggcactgactgcccacctgttcgtgtcaatggaatcggagggctacctggtggcaaggtcaagctgtctggctccatcagcagtcagtacttgagtgccttgctgatggctgctcctttggctcttggggatgtggagattgaaatcattgataaattaatctccattccctacgtcgaaatgacattgagattgatggagcgttttggtgtgaaagcagagcattctgatagctgggacagattctacattaagggaggtcaaaaatacaagtcccctaaaaatgcctatgttgaaggtgatgcctcaagcgcaagctatttcttggctggtgctgcaattactggagggactgtgactgtggaaggttgtggcaccaccagtttgcagggtgatgtgaagtttgctgaggtactggagatgatgggagcgaaggttacatggaccgagactagcgtaactgttactggcccaccgcgggagccatttgggaggaaacacctcaaggcgattgatgtcaacatgaacaagatgcctgatgtcgccatgactcttgctgtggttgccctctttgccgatggcccgacagccatcagagacgtggcttcctggagagtaaaggagaccgagaggatggttgcgatccggacggagctaaccaagctgggagcatctgttgaggaagggccggactactgcatcatcacgccgccggagaagctgaacgtgacggcgatcgacacgtacgacgaccacaggatggccatggccttctcccttgccgcctgtgccgaggtccccgtggccatccgggaccctgggtgcacccggaagaccttccccgactacttcgatgtgctgagcactttcgtcaagaattaa

SEQ ID NO: 8 - protein, 506 aa, Zea mays
MAAMATKAAAGTVSLDLAAPSRRHHRPSSARPPARPAVRGLRAPGRRVIAAPPAAAAAAAVQAGAEEIVLQPIKEISGTVKLPGSKSLSNRILLLAALSEGTTVVDNLLNSEDVHYMLGALRTLGLSVEADKAAKRAVVVGCGGKFPVEDSKEEVQLFLGNAGIAMRSLTAAVTAAGGNATYVLDGVPRMRERPIGDLVVGLKQLGADVDCFLGTDCPPVRVNGIGGLPGGKVKLSGSISSQYLSALLMAAPLALGDVEIEIIDKLISIPYVEMTLRLMERFGVKAEHSDSWDRFYIKGGQKYKSPKNAYVEGDASSASYFLAGAAITGGTVTVEGCGTTSLQGDVKFAEVLEMMGAKVTWTETSVTVTGPPREPFGRKHLKAIDVNMNKMPDVAMTLAVVALFADGPTAIRDVASWRVKETERMVAIRTELTKLGASVEEGPDYCIITPPEKLNVTAIDTYDDHRMAMAFSLAACAEVPVAIRDPGCTRKTFPDYFDVLSTFVKN

SEQ ID NO: 9 - protein, 444 aa, Zea mays
AGAEEIVLQPIKEISGTVKLPGSKSLSNRILLLAALSEGTTVVDNLLNSEDVHYMLGALRTLGLSVEADKAAKRAVVVGCGGKFPVEDSKEEVQLFLGNAGIAMRSLTAAVTAAGGNATYVLDGVPRMRERPIGDLVVGLKQLGADVDCFLGTDCPPVRVNGIGGLPGGKVKLSGSISSQYLSALLMAAPLALGDVEIEIIDKLISIPYVEMTLRLMERFGVKAEHSDSWDRFYIKGGQKYKSPKNAYVEGDASSASYFLAGAAITGGTVTVEGCGTTSLQGDVKFAEVLEMMGAKVTWTETSVTVTGPPREPFGRKHLKAIDVNMNKMPDVAMTLAVVALFADGPTAIRDVASWRVKETERMVAIRTELTKLGASVEEGPDYCIITPPEKLNVTAIDTYDDHRMAMAFSLAACAEVPVAIRDPGCTRKTFPDYFDVLSTFVKN

SEQ ID NO: 10 - protein, 444 aa, Zea mays
AGAEEIVLQPIKEISGTVKLPGSKSLSNRILLLAALSEGTTVVDNLLNSEDVHYMLGALRTLGLSVEADKAAKRAVVVGCGGKFPVEDSKEEVQLFLGNAGIAMRSLTAAVTAAGGNATYVLDGVSRMRERPIGDLVVGLKQLGADVDCFLGTDCPPVRVNGIGGLPGGKVKLSGSISSQYLSALLMAAPLALGDVEIEIIDKLISIPYVEMTLRLLERFGVKAEHSDSWDRFYIKGGQKYKSPKNAYVEGDASSASYFLAGAAITGGTVTVEGCGTTSLQGDVKFAEVLEMMGARVTWTETSVTVTGPPREPFGRKHLKAIDVNMNKMPDVAMTLAVVALFADGPTAIRDVASWRVKETERMVAIRTELTKLGASVEEGPDYCIITPPEKLNVTAIDTYDDHRMAMAFSLAACAEVPVAIRDPGCTRKTFPDYFDVLSTFVKN

SEQ ID NO: 11 - protein, 444 aa, Zea mays
AGAEEIVLQPIKEISGTVKLPGSKSLSNRILLLAALSEGTTVVDNLLNSEDVHYMLGALRTLGLSVEADKAAKRAVVVGCGGKFPVEDSKEEVQLFLGNAGIAMRSLTAAVTAAGGNATYVLDGVSRMRERPIGDLVVGLKQLGADVDCFLGTDCPPVRVNGIGGLPGGKVKLSGSISSQYLSALLMAAPLALGDVEIEIIDKLISIPYVEMTLRLMERFGVKAEHSDSWDRFYIKGGQKYKSPKNAYVEGDASSASYFLAGAAITGGTVTVEGCGTTSLQGDVKFAEVLEMMGARVTWTETSVTVTGPPREPFGRKHLKAIDVNMNKMPDVAMTLAVVALFADGPTAIRDVASWRVKETERMVAIRTELTKLGASVEEGPDYCIITPPEKLNVTAIDTYDDHRMAMAFSLAACAEVPVAIRDPGCTRKTFPDYFDVLSTFVKN

SEQ ID NO: 12 - protein, 444 aa, Zea mays
AGAEEIVLQPIKEISGTVKLPGSKSLSNRILLLAALSEGTTVVDNLLNSEDVHYMLGALRTLGLSVEADKAAKRAVVVGCGGKFPVEDSKEEVQLFLGNAGIAMRPLTAAVTAAGGNATYVLDGVSRMRERPIGDLVVGLKQLGADVDCFLGTDCPPVRVNGIGGLPGGKVKLSGSISSQYLSALLMAAPLALGDVEIEIIDKLISIPYVEMTLRLMERFGVKAEHSDSWDRFYIKGGQKYKSPKNAYVEGDASSASYFLAGAAITGGTVTVEGCGTTSLQGDVKFAEVLEMMGARVTWTETSVTVTGPPREPFGRKHLKAIDVNMNKMPDVAMTLAVVALFADGPTAIRDVASWRVKETERMVAIRTELTKLGASVEEGPDYCIITPPEKLNVTAIDTYDDHRMAMAFSLAACAEVPVAIRDPGCTRKTFPDYFDVLSTFVKN

SEQ ID NO: 13 - protein, 444 aa, Zea mays
AGAEEIVLQPIKEISGTVKLPGSKSLSNRILLLAALSEGTTVVDNLLNSEDVHYMLGALRTLGLSVEADKAAKRAVVVGCGGKFPVEDSKEEVQLFLGNAGTAMRSLTAAVTAAGGNATYVLDGVSRMRERPIGDLVVGLKQLGADVDCFLGTDCPPVRVNGIGGLPGGKVKLSGSISSQYLSALLMAAPLALGDVEIEIIDKLISIPYVEMTLRLMERFGVKAEHSDSWDRFYIKGGQKYKSPKNAYVEGDASSASYFLAGAAITGGTVTVEGCGTTSLQGDVKFAEVLEMMGARVTWTETSVTVTGPPREPFGRKHLKAIDVNMNKMPDVAMTLAVVALFADGPTAIRDVASWRVKETERMVAIRTELTKLGASVEEGPDYCIITPPEKLNVTAIDTYDDHRMAMAFSLAACAEVPVAIRDPGCTRKTFPDYFDVLSTFVKN

SEQ ID NO: 14 - protein, 444 aa, Zea mays
AGAEEIVLQPIKEISGTVKLPGSKSLSNRILLLAALSEGTTVVDNLLNSEDVHYMLGALRTLGLSVEADKAAKRAVVVGCGGKFPVEDSKEEVQLFLGNAGTAMRPLTAAVTAAGGNATYVLDGVSRMRERPIGDLVVGLKQLGADVDCFLGTDCPPVRVNGIGGLPGGKVKLSGSISSQYLSALLMAAPLALGDVEIEIIDKLISIPYVEMTLRLMERFGVKAEHSDSWDRFYIKGGQKYKSPKNAYVEGDASSASYFLAGAAITGGTVTVEGCGTTSLQGDVKFAEVLEMMGARVTWTETSVTVTGPPREPFGRKHLKAIDVNMNKMPDVAMTLAVVALFADGPTAIRDVASWRVKETERMVAIRTELTKLGASVEEGPDYCIITPPEKLNVTAIDTYDDHRMAMAFSLAACAEVPVAIRDPGCTRKTFPDYFDVLSTFVKN

SEQ ID NO: 15 - protein, 444 aa, Zea mays
AGAEEIVLQPIKEISGTVKLPGSKSLSNRILLLAALSEGTTVVDNLLNSEDVHYMLGALRTLGLSVEADKAAKRAVVVGCGGKFPVEDSKEEVQLFLGNAGIAMRSLTAAVTAAGGNATYVLDGVSRMRERPIGDLVVGLKQLGADVDCFLGTDCPPVRVNGIGGLPGGKVKLSGSISSQYLSALLMAAPLALGDVEIEIIDKLISIPYVEMTLRLMERFGVKAEHSDSWDRFYIKGGQKYKSPKNAYVEGDASSASYFLAGAAITGGTVTVEGCGTTSLQGDVKFAEVLEMMGAKVTWTETSVTVTGPPREPFGRKHLKAIDVNMNKMPDVAMTLAVVALFADGPTAIRDVASWRVKETERMVAIRTELTKLGASVEEGPDYCIITPPEKLNVTAIDTYDDHRMAMAFSLAACAEVPVAIRDPGCTRKTFPDYFDVLSTFVKN

SEQ ID NO: 16 - protein, 444 aa, Zea mays
AGAEEIVLQPIKEISGTVKLPGSKSLSNRILLLAALSEGTTVVDNLLNSEDVHYMLGALRTLGLSVEADKAAKRAVVVGCGGKFPVEDSKEEVQLFLGNAGIAMRSLTAAVTAAGGNATYVLDGVPRMRERPIGDLVVGLKQLGADVDCFLGTDCPPVRVNGIGGLPGGKVKLSGSISSQYLSALLMAAPLALGDVEIEIIDKLISIPYVEMTLRLMERFGVKAEHSDSWDRFYIKGGQKYKSPKNAYVEGDASSASYFLAGAAITGGTVTVEGCGTTSLQGDVKFAEVLEMMGARVTWTETSVTVTGPPREPFGRKHLKAIDVNMNKMPDVAMTLAVVALFADGPTAIRDVASWRVKETERMVAIRTELTKLGASVEEGPDYCIITPPEKLNVTAIDTYDDHRMAMAFSLAACAEVPVAIRDPGCTRKTFPDYFDVLSTFVKN

SEQ ID NO: 17 - protein, 444 aa, Zea mays
AGAEEIVLQPIKEISGTVKLPGSKSLSNRILLLAALSEGTTVVDNLLNSEDVHYMLGALRTLGLSLEADKAAKRAVVVGCGGKFPVEDSKEEVQLFLGNAGIALRSLTAAVTAAGGNATYVLDGVPRMRERPIGDLVVGLKQLGADVDCFLGTDCPPVRVNGIGGLPGGKVKLSGSISSQYLSALLMAAPLALGDVEIEIIDKLISIPYVEMTLRLMERFGVKAEHSDSWDRFYIKGGQKYKSPKNAYVEGDASSASYFLAGAAITGGTVTVEGCGTTSLQGDVKFAEVLEMMGAKVTWTETSVTVTGPPREPFGRKHLKAIDVNMNKMPDVAMTLAVVALFADGPTAIRDVASWRVKETERMVAIRTELTKLGASVEEGPDYCIITPPEKLNVTAIDTYDDHRMAMAFSLAACAEVPVAIRDPGCTRKTFPDYFDVLSTFVKN

SEQ ID NO: 18 - protein, 444 aa, Zea mays
AGAEEIVLQPIKEISGTVKLPGSKSLSNRILLLAALSEGTTVVDNLLNSEDVHYMLGALRTLGLSVEADKAAKRAVVVGCGGKFPVEDSKEEVQLFLGNAGIAMRSLSAAVTAAGGNATYVLDGVPRMRERPIGDLVVGLKQLGADVDCFLGTDCPPVRVNGIGGLPGGKVKLSGSISSQYLSALLMAAPLALGDVEIEIIDKLISIPYVEMTLRLMERFGVKAEHSDSWDRFYIKGGQKYKSPKNAYVEGDASSASYFLAGAAITGGTVTVEGCGTTSLQGDVKFAEVLEMMGAKVTWTETSVTVTGPPREPFGRKHLKAIDVNMNKMPDVAMTLAVVALFADGPTAIRDVASWRVKETERMVAIRTELTKLGASVEEGPDYCIITPPEKLNVTAIDTYDDHRMAMAFSLAACAEVPVAIRDPGCTRKTFPDYFDVLSTFVKN

SEQ ID NO: 19 - protein, 444 aa, Zea mays
AGAEEIVLQPIKEISGTVKLPGSKSLSNRILLLAALSEGTTVVDNLLNSEDVHYMLGALRTLGLSVEADKAAKRAVVVGCGGKFPVEDSKEEVQLFLGNAGIAMRSLTAAVTAAGGNATYVLDGVPRMRERPIGDLVVGLKQLGADVDCVLGTDCPPVRVNGIGGLPGGKVKLSGSISSQYLSALLMAAPLALGDVEIEIIDKLISIPYVEMTLRLMERFGVKAEHSDSWDRFYIKGGQKYKSPKNAYVEGDASSASYFLAGAAITGGTVTVEGCGTTSLQGDVKFAEVLEMMGAKVTWTETSVTVTGPPREPFGRKYLKAIDVNMNKMPDVAMTLAVVALFADGPTAIRDVASWRVKETERMVAIRTELTKLGASVEEGPDYCIITPPEKLNVTAIDTYDDHRMAMAFSLAACAEVPVAIRDPGCTRKTFPDYFDVLSTFVKN

SEQ ID NO: 20 - protein, 444 aa, Zea mays
AGAEEIVLQPIKEISGTVKLPGSKSLSNRILLLAALSEGTTVVDNLLNSEDVHYMLGALRTLGLSVEADKAAKRAVVVGCGGKFPVEDSKEEVQLFLGNAGIAMRSLTAAVTAAGVNATYVLDGVPRMRERPIGDLVVGLKQLGADVDCFLGTDCPPVRVNGIGGLPGGKVKLSGSISSQYLSALLMAAPLALGDVEIEIIDKLISIPYVEMTLRLMERFGVKAEHSDSWDRFYIKGGQKYKSPKNAYVEGDASSASYFLAGAAITGGTVTVEGCGTTSLQGDVKFAEVLEMMGAKVTWTETSVTVTGPPREPFGRKHLKAIDVNMNKMPDVAMTLAVVALFADGPTAIRDVASWRVKETERMVAIRTELTKLGASVEEGPDYCIITPPEKLNVTAIDTYDDHRMAMAFSLAACAEVPVAIRDPGCTRKTFPDYFDVLSTFVKN

SEQ ID NO: 21 - protein, 444 aa, Zea mays
AGAEEIVLQPIKEISGTVKLPGSKSLSNRILLLAALSEGTTVVDNLLNSEDVHYMLGALRTLGLSVEADKAAKRAVVVGCGGKFPVEDSKEEVQLFLGNAGIAMRSLTAAVTAAGGNATYVLDGVPRMRERPIGDLVVGLKQLGADVDCFLGTDCPPVRVNGIGGLPGGKVKLSGSVSSQYLSALLMAAPLALGDVEIEIIDKLISIPYVEMTLRLMERFGVKAEHSDSWDRFYIKGGQKYKSPKNAYVEGDASSASYFLAGAAITGGTVTVEGCGTTSLQGDVKFAEVLEMMGAKVTWTETSVTVTGPPREPFGRKHLKAIDVNMNKMPDVAMTLAVVALFADGPTAIRDVASWRVKETERMVAIRTELTKLGASVEEGPDYCIITPPEKLNVTAIDTYDDHRMAMAFSLAACAEVPVAIRDPGCTRKTFPDYFDVLSTFVKN

SEQ ID NO: 22 - protein, 444 aa, Zea mays
AGAEEIVLQPIKEISGTVKLPGSKSLSNRILLLAALSEGTTVVDNLLNSEDVHYMLGALRTLGLSVEADKAAKRAVVVGCGGKFPVEDSKEEVQLFLGNAGIAMRSLTAAVTAAGGNATYVLDGVLRMRERPIGDLVVGLKQLGADVDCFLGTDCPPVRVNGIGGLPGGKVKLSGSISSQYLSALLMAAPLALGDVEIEIIDKLISIPYVEMTLRLMERFGVKAEHSDSWDRFYIKGGQKYKSPKNAYVEGDASSASYFLAGAAITGGTVTVEGCGTTSLQGDVKFAEVLEMMGAKVTWTETSVTVTGPPREPFGRKHLKAIDVNMNKMPDVAMTLAVVALFADGPTAIRDVASWRVKETERMVAIRTELTKLGASVEEGPDYCIITPPEKLNVTAIDTYDDHRMAMAFSLAACAEVPVAIRDPGCTRKTFPDYFDVLSTFVKN

SEQ ID NO: 23 - protein, 444 aa, Zea mays
AGAEEIVLQPIKEISGTVKLPGSKSLSNRILLLAALSEGTTVVDNLLNSEDVHYMLGALRTLGLSVEADKAAKRAVVVGCGGKFPVEDSKEEVQLFLGNAGTAMRSLTVAVTAAGGNATYVLDGVPRMRERPIGDLVVGLKQLGADVDCFLGTDCPPVRVNGKGGLPGGKVKLSGSISSQYLSALLMAAPLALGDVEIEIIDKLISIPYVEMTLRLMERFGVKAEHSDSWDRFYIKGGQKYKSPKNAYVEGDASSASYFLAGAAITGGTVTVEGCGTTSLQGDVKFAEVLEMMGAKVTWTETSVTVTGPPREPFGRKHLKAIDVNMNKMPDVAMTLAVVALFADGPTAIRDVASWRVKETERMVAIRTELTKLGASVEEGPDYCIITPPEKLNVTAIDTYDDHRMAMAFSLAACAEVPVAIRDPGCTRKTFPDYFDVLSTFVKN

(19) United States
(12) Patent Application Publication
(10) Pub. No.: US 2026/0275304 A1
(43) Pub. Date: Sep. 17, 2026

(54) METHODS AND COMPOSITIONS FOR GLYPHOSATE TOLERANCE IN PLANTS

(71) Applicants: Monsanto Technology LLC, St. Louis, MO (US); Board of Regents, The University of Texas System, Austin, TX (US)

(72) Inventors: Hal Alper, Austin, TX (US); Shirley X. Guo, Chesterfield, MO (US); Sergey Ivashuta, Ballwin, MO (US); Clayton T. Larue, Chesterfield, MO (US); Kevin B. Reed, Austin, TX (US)

(21) Appl. No.: 19/473,909

(22) PCT Filed: May 29, 2024
(86) PCT No.: PCT/US2024/031423

§ 371 Date: Oct. 9, 2025

Related U.S. Application Data
(60) Provisional application No. 63/511,073, filed on Jun. 29, 2023.

(51) Int. Cl. C12N 9/10; A01N 57/20; A01P 13/02; C12N 15/81; C12N 15/82; C12R 1/865

(52) U.S. Cl. CPC C12N 9/1092; A01N 57/20; A01P 13/02; C12N 15/81; C12N 15/8275; C12R 2001/865; C12Y 205/01019

(57) ABSTRACT

The present disclosure relates to novel methods and compositions for conferring tolerance to glyphosate to plants. The present disclosure also provides glyphosate-tolerant plants, seeds, tissues, cells, and plant parts comprising modified EPSP synthases and recombinant DNA molecules encoding modified EPSP synthases, as well as methods of producing the same and the use thereof.

### CROSS-REFERENCE TO RELATED APPLICATIONS

This application claims the benefit under 35 U.S.C. § 119 (e) of U.S. provisional application Ser. No. 63/511,073, filed on Jun. 29, 2023, which is herein incorporated by reference in its entirety.

### FIELD OF THE INVENTION

The present disclosure relates to the field of biotechnology. More specifically, the disclosure relates to recombinant DNA molecules encoding engineered 5-enolpyruvylshikimate-3-phosphate synthase (EPSPS) enzymes that provide tolerance to the herbicide glyphosate. The present disclosure also provides methods of engineering enzymes with improved herbicide tolerance and enzymatic efficiency.

### INCORPORATION OF SEQUENCE LISTING

The sequence listing that is contained in the file named MONS540WO_ST26.xml, which is 41.3 kilobytes (measured in MS-WINDOWS) and created on May 22, 2024, is filed herewith by electronic submission and incorporated herein by reference.

### BACKGROUND OF THE INVENTION

Glyphosate, or N-phosphonomethylglycine, is a broad-spectrum, foliar-applied herbicide that inhibits 5-enolpyruvylshikimate-3-phosphate synthase (EPSP synthase or EPSPS) in plants. EPSPS is part of the shikimate pathway used in plants for the biosynthesis of folates and aromatic amino acids. EPSP synthases from different organisms have been divided into two classes based on glyphosate sensitivity. All plants have class I EPSP synthases, which are glyphosate-sensitive. Glyphosate tolerant crops have been primarily produced using the glyphosate-insensitive class II EPSPS from Agrobacterium sp. strain CP4. Glyphosate tolerance in crops permits the use of glyphosate to control weeds while maintaining crop yield. The T102I-P106S double mutation of the class I EPSPS from maize has been shown to confer insensitivity to glyphosate; however, these resistance-conferring mutations also reduce the catalytic efficiency of this essential enzyme and thus lead to reduced plant growth under native expression. Thus, there is a need for novel plant EPSPS variants having improved tolerance to glyphosate and high catalytic efficiency as well as methods of producing and using the same.

### SUMMARY OF THE INVENTION

The present disclosure provides a recombinant DNA molecule encoding a glyphosate-tolerant 5-enol-pyruvyl-shikimate-3-phosphate synthase (EPSPS), wherein the EPSPS comprises at least a first amino acid substitution combination selected from the group consisting of: T102I-P106S-P126S-K296R, T102I-P106S-P126S, T102I-P106S-K296R, T102I-P106S-P126S-M217L-K296R, T102I-P106S-V66L-M104L, T102I-P106S-T108S, T102I-P106S-F150V-H318Y, T102I-P106S-P126L, T102I-P106S-G116V, T102I-P106S-1177V, and P106S-A109V-I163K, wherein the position of the amino acid substitution is relative to the position of the amino acid sequence provided as SEQ ID NO:6. In certain embodiments, the EPSPS is a maize EPSPS. In some embodiments, the EPSPS confers increased tolerance to glyphosate or increased enzymatic efficiency as compared to an EPSPS lacking the combination. In other embodiments, the EPSPS comprises at least two of the substitution combinations.

The present disclosure also provides a glyphosate-tolerant EPSPS comprising at least a first amino acid substitution combination selected from the group consisting of: T102I-P106S-P126S-K296R, T102I-P106S-P126S, T102I-P106S-K296R, T102I-P106S-P126S-M217L-K296R, T102I-P106S-V66L-M104L, T102I-P106S-T108S, T102I-P106S-F150V-H318Y, T102I-P106S-P126L, T102I-P106S-G116V, T102I-P106S-1177V, and P106S-A109V-1163K, wherein the position of the amino acid substitution is relative to the position of the amino acid sequence provided as SEQ ID NO:6. In particular embodiments, the EPSPS confers increased tolerance to glyphosate or increased enzymatic efficiency as compared to an EPSPS lacking the combination, or as compared to an EPSPS containing only T102I-P106S. In further embodiments, the EPSPS comprises at least two of the substitution combinations.

The present disclosure additionally provides a plant, seed, cell, plant part, or commodity product comprising a recombinant DNA molecule encoding a glyphosate-tolerant 5-enol-pyruvyl-shikimate-3-phosphate synthase (EPSPS), wherein the EPSPS comprises at least a first amino acid substitution combination selected from the group consisting of: T102I-P106S-P126S-K296R, T102I-P106S-P126S, T102I-P106S-K296R, T102I-P106S-P126S-M217L-K296R, T102I-P106S-V66L-M104L, T102I-P106S-T108S, T102I-P106S-F150V-H318Y, T102I-P106S-P126L, T102I-P106S-G116V, T102I-P106S-1177V, and P106S-A109V-1163K, wherein the position of the amino acid substitution is relative to the position of the amino acid sequence provided as SEQ ID NO:6. In certain embodiments, the plant exhibits increased glyphosate tolerance when compared to a plant lacking the combination. In some embodiments, the plant is a corn, soy, cotton, canola, wheat, rice, alfalfa, sugar beet, oilseed rape or sugar cane plant.

The present disclosure further provides a transgenic maize plant comprising a recombinant DNA molecule encoding a glyphosate-tolerant 5-enol-pyruvyl-shikimate-3-phosphate synthase (EPSPS), wherein the EPSPS comprises at least a first amino acid substitution combination selected from the group consisting of: T102I-P106S-P126S-K296R, T102I-P106S-P126S, T102I-P106S-K296R, T102I-P106S-P126S-M217L-K296R, T102I-P106S-V66L-M104L, T102I-P106S-T108S, T102I-P106S-F150V-H318Y, T102I-P106S-P126L, T102I-P106S-G116V, T102I-P106S-1177V, and P106S-A109V-1163K, wherein the position of the amino acid substitution is relative to the position of the amino acid sequence provided as SEQ ID NO: 6, and wherein the EPSPS confers glyphosate tolerance to the transgenic maize plant. In additional embodiments, the glyphosate tolerance is greater than the glyphosate tolerance conferred by an EPSPS that comprises only a T102I-P106S substitution.

The present disclosure also provides a seed, cell or plant part of a transgenic maize plant comprising a recombinant DNA molecule encoding a glyphosate-tolerant 5-enol-pyruvyl-shikimate-3-phosphate synthase (EPSPS), wherein the EPSPS comprises at least a first amino acid substitution combination selected from the group consisting of: T102I-P106S-P126S-K296R, T102I-P106S-P126S, T102I-P106S-K296R, T102I-P106S-P126S-M217L-K296R, T102I-P106S-V66L-M104L, T102I-P106S-T108S, T102I-P106S-F150V-H318Y, T102I-P106S-P126L, T102I-P106S-G116V, T102I-P106S-1177V, and P106S-A109V-1163K, wherein the position of the amino acid substitution is relative to the position of the amino acid sequence provided as SEQ ID NO: 6, and wherein the EPSPS confers glyphosate tolerance to the transgenic maize plant, wherein the seed, cell or plant part comprises the recombinant DNA molecule.

The present disclosure additionally provides a method for conferring glyphosate tolerance to a plant comprising expressing in the plant a glyphosate-tolerant EPSPS comprising at least a first amino acid substitution combination selected from the group consisting of: T102I-P106S-P126S-K296R, T102I-P106S-P126S, T102I-P106S-K296R, T102I-P106S-P126S-M217L-K296R, T102I-P106S-V66L-M104L, T102I-P106S-T108S, T102I-P106S-F150V-H318Y, T102I-P106S-P126L, T102I-P106S-G116V, T102I-P106S-1177V, and P106S-A109V-I163K, wherein the position of the amino acid substitution is relative to the position of the amino acid sequence provided as SEQ ID NO:6. In particular embodiments, the method comprises introducing a recombinant DNA molecule encoding a glyphosate-tolerant 5-enol-pyruvyl-shikimate-3-phosphate synthase (EPSPS), wherein the EPSPS comprises at least a first amino acid substitution combination selected from the group consisting of: T102I-P106S-P126S-K296R, T102I-P106S-P126S, T102I-P106S-K296R, T102I-P106S-P126S-M217L-K296R, T102I-P106S-V66L-M104L, T102I-P106S-T108S, T102I-P106S-F150V-H318Y, T102I-P106S-P126L, T102I-P106S-G116V, T102I-P106S-1177V, and P106S-A109V-1163K, wherein the position of the amino acid substitution is relative to the position of the amino acid sequence provided as SEQ ID NO: 6, into the genome of the plant or a progenitor thereof.

Furthermore, the present disclosure provides a method for producing a glyphosate-tolerant EPSPS comprising introducing into a nucleic acid molecule encoding a plant EPSPS at least a first amino acid substitution combination selected from the group consisting of: T102I-P106S-P126S-K296R, T102I-P106S-P126S, T102I-P106S-K296R, T102I-P106S-P126S-M217L-K296R, T102I-P106S-V66L-M104L, T102I-P106S-T108S, T102I-P106S-F150V-H318Y, T102I-P106S-P126L, T102I-P106S-G116V, T102I-P106S-1177V, and P106S-A109V-I163K, wherein the position of the amino acid substitution is relative to the position of the amino acid sequence provided as SEQ ID NO:6. In certain embodiments, the introducing is carried out in vitro. In other embodiments, the introducing is carried out in planta. In some embodiments, the introducing comprises use of at least a first site-specific endonuclease. In additional embodiments, the method comprises introducing at least two of the substitution combinations into the EPSPS.

The present disclosure also provides a method for controlling weeds in a plant growth area, comprising contacting a plant growth area comprising a transgenic maize plant comprising a recombinant DNA molecule encoding a glyphosate-tolerant 5-enol-pyruvyl-shikimate-3-phosphate synthase (EPSPS), wherein the EPSPS comprises at least a first amino acid substitution combination selected from the group consisting of: T102I-P106S-P126S-K296R, T102I-P106S-P126S, T102I-P106S-K296R, T102I-P106S-P126S-M217L-K296R, T102I-P106S-V66L-M104L, T102I-P106S-T108S, T102I-P106S-F150V-H318Y, T102I-P106S-P126L, T102I-P106S-G116V, T102I-P106S-1177V, and P106S-A109V-1163K, wherein the position of the amino acid substitution is relative to the position of the amino acid sequence provided as SEQ ID NO: 6, and wherein the EPSPS confers glyphosate tolerance to the transgenic maize plant, or a seed of a transgenic maize plant comprising a recombinant DNA molecule encoding a glyphosate-tolerant 5-enol-pyruvyl-shikimate-3-phosphate synthase (EPSPS), wherein the EPSPS comprises at least a first amino acid substitution combination selected from the group consisting of: T102I-P106S-P126S-K296R, T102I-P106S-P126S, T102I-P106S-K296R, T102I-P106S-P126S-M217L-K296R, T102I-P106S-V66L-M104L, T102I-P106S-T108S, T102I-P106S-F150V-H318Y, T102I-P106S-P126L, T102I-P106S-G116V, T102I-P106S-1177V, and P106S-A109V-I163K, wherein the position of the amino acid substitution is relative to the position of the amino acid sequence provided as SEQ ID NO:6, and wherein the EPSPS confers glyphosate tolerance to the transgenic maize plant, wherein the seed, cell or plant part comprises the recombinant DNA molecule, with glyphosate, wherein the plant or seed is tolerant to glyphosate, and wherein weeds are controlled in the plant growth area.

Additionally, the present disclosure provides a yeast cell comprising a knockout of native aro1 function, and further comprising a recombinant DNA molecule comprising a heterologous promoter operably linked to an aro1 coding sequence comprising a D731A mutation, wherein the yeast cell is not able to grow on media without aromatic amino acids. In certain embodiments, the yeast cell further comprises a heterologous nucleic acid molecule encoding an EPSPS, wherein the yeast cell is able to grow on media without aromatic amino acids. In some embodiments, the EPSPS is a glyphosate-tolerant maize EPSPS. In other embodiments, the yeast cell is a Saccharomyces cerevisiae cell.

The present disclosure further provides a method for identifying a glyphosate tolerant EPSPS having improved growth, improved glyphosate tolerance or improved enzymatic efficiency when compared to wild-type EPSPS, comprising the steps of: a) obtaining a yeast cell comprising a knockout of native aro1 function, and further comprising a recombinant DNA molecule comprising a heterologous promoter operably linked to an aro1 coding sequence comprising a D731A mutation, wherein the yeast cell is not able to grow on media without aromatic amino acids; and b) identifying the yeast cell as capable of growing in the presence of glyphosate and therefore comprising a glyphosate tolerant EPSPS having improved growth, improved glyphosate tolerance or improved enzymatic efficiency when compared to wild-type EPSPS. In further embodiments, the method comprises obtaining a population of yeast cells collectively comprising a plurality of heterologous nucleic acid molecules encoding mutant EPSPS proteins and identifying at least one of the yeast cells as having improved growth, improved glyphosatc tolerance or improved enzymatic efficiency. In some embodiments, the method further comprises cloning the nucleic acid molecule encoding the glyphosate tolerant EPSPS from the yeast cell or a progeny thereof.

The present disclosure also provides a method of identifying a plant, seed, cell, or plant part as comprising glyphosate tolerance, the method comprising applying glyphosate to the plant, seed, cell, or plant part that comprises a recombinant DNA molecule encoding a glyphosate-tolerant 5-enol-pyruvyl-shikimate-3-phosphate synthase (EPSPS), wherein the EPSPS comprises at least a first amino acid substitution combination selected from the group consisting of: T102I-P106S-P126S-K296R, T102I-P106S-P126S, T102I-P106S-K296R, T102I-P106S-P126S-M217L-K296R, T102I-P106S-V66L-M104L, T102I-P106S-T108S, T102I-P106S-F150V-H318Y, T102I-P106S-P126L, T102I-P106S-G116V, T102I-P106S-1177V, and P106S-A109V-I163K, wherein the position of the amino acid substitution is relative to the position of the amino acid sequence provided as SEQ ID NO:6. In some embodiments, the method further comprises applying the glyphosate to a population of plants, seeds, cells, or plant parts. In other embodiments, the method comprises applying the glyphosate to a culture of cells.

### BRIEF DESCRIPTION OF THE DRAWINGS

FIG. 1: Growth of wild-type S. cerevisiae BY4741 (Panel A) and S. cerevisiae strain sKR-024-Ptef-EPSPS (Panel B) in 0, 5 mM and 10 mM glyphosate, and growth of S. cerevisiae with Zea mays EPSPS, Zea mays EPSPS-P102S, Zea mays EPSPS-T106I and Zea mays EPSPS-T102I-P106S in 5 mM glyphosate (Panel C).

FIG. 2: Schematic of generation of a tetrafunctional ScARO1 by removing EPSPS function. Panel A: Overview of the shikimate pathway in plants. Glyphosate competitively inhibits EPSP synthase to prevent the creation of aromatic amino acids (AAAs). Panel B: Overview of the wild-type pentafunctional ARO1 in yeast. Mutation D731A successfully abolished catalytic function of EPSPS portion while keeping other domains intact and functional.

FIG. 3: Construction of the Δaro1, aro1 D731A mutant and aro1 D731A mutant-ZmEPSPS strains, and growth of the resulting strains on solid media with and without aromatic amino acids.

FIG. 4: Development of a synthetic yeast model enables growth-dependence of a heterologous EPSPS. Panel A: Schematic of synthetic yeast model with two plasmids. Panel B: Synthetic yeast host either expressing ZmEPSPS (green curve) or lacking ZmEPSPS (grey curve). Data represents the average of 3 biological triplicates picked from individual colonies at random and the shaded error bar region represents the S.E.

FIG. 5: Schematic of S. cerevisiae strain sKR-024 containing a red fluorescent protein (RFP) cassette and TIPS cultured in yeast minimal media containing 0, 2.5, 5, 7.5, or 10 mM glyphosate, and resultant flow cytometry analysis of RFP fluorescence intensity.

FIG. 6: Tuning the selection pressure of synthetic yeast host via expression optimization. Panel A: Specific Growth Rate for EPSPS and TIPS when expressed under varying promoter strengths (promoter series) in 0 mM glyphosate. Panel B: Resulting growth rates in 5 mM glyphosate. Differences in growth rate between EPSPS and TIPS enable a selection window. Core1p promoter was selected due to more consistent growth characteristics and differences between wild-type and mutant EPSP synthases. Panel C: Growth curves of well-studied mutants P106S and TIPS compared to wild-type EPSPS variants grown in 0.5 mM glyphosate in the synthetic yeast model, near the maximum value of what is seen in plant tissue during field application. Glyphosate tolerance is highest in TIPS. Panel D: Variants grown in 0 mM glyphosate in the synthetic yeast model. The growth deficit caused by TIPS is evident. Absorbance at 600 nm was measured on a Tecan plate reader over time. Each column represents the average of 3 biological replicates, where specific growth rate was calculated individually for each replicate. Error bars represent the standard deviation.

FIG. 7: Growth of novel glyphosate resistance mutants starting from corn EPSPS and corn EPSPS TIPS placed under selection separately to observe their independent outcomes towards the goal(s) of improved glyphosate-resistance and/or improved overall catalytic activity.

FIG. 8: Growth of EPSPS low and medium mutation frequency mutagenesis libraries in 0 mM, 2.5 mM and 5 mM glyphosate.

FIG. 9: Growth of EPSPS TIPS low and medium mutation frequency mutagenesis libraries in 0 mM, 2.5 mM and 5 mM glyphosate.

FIG. 10: Mutational outcomes from EPSPS and TIPS background low and medium mutation frequency libraries.

FIG. 11: Mutational outcomes from EPSPS and TIPS background low and medium mutation frequency libraries.

FIG. 12: Schematic of multiple outgrowths of a TIPS (med) culture, with no exposure to any strong selections, a TIPS (med) culture that had a single outgrowth in 2.5 mM glyphosate, and a TIPS (med) culture with oscillating selection ending on 2.5 mM glyphosate.

FIG. 13: Mutational outcomes from TIPS (med) culture with no exposure to any strong selections.

FIG. 14: Mutational outcomes from TIPS (med) culture that had a single outgrowth in 2.5 mM glyphosate.

FIG. 15: Mutational outcomes from TIPS (med) culture with oscillating selection ending on 2.5 mM glyphosate.

FIG. 16: Outcomes of the optimized dual-trait selection pressure selections starting from EPSPS background (Panel A) and TIPS background (Panel B). Top hits from each selection stage are shown.

FIG. 17: A comparison of example variants resulting from either mono- or dual-trait selection pressure. Panel A: EPSPS evolution entry point mutation pools. Each point is the average growth rate of biological triplicates grown in either 0 mM or 0.25 mM glyphosate, the level at which wild-type EPSPS cannot grow. Panel B: TIPS evolution entry point mutation pools. Each point is the average growth rate of biological triplicates grown in either 0 mM or 5 mM glyphosate, a level at which high glyphosate tolerance can be evaluated.

FIG. 18: Growth of EPSPS, TIPS and each combination of mutant TIPS-P126S-M217L-K296R in 0 mM, 2.5 mM and 5 mM glyphosate.

FIG. 19: Growth of EPSPS, TIPS, TIPS-P126S, TIPS-K296R and TIPS-P126S-K296R in 0 mM, 2.5 mM and 5 mM glyphosate.

FIG. 20: Growth of EPSPS, G101A, G101A-P126S, G101A-K296R and G101A-P126S-K296R in 0 mM and 5 mM glyphosate.

FIG. 21: Growth of EPSPS, T102I, T102I-P126S and T102I-K296R in 0 mM and 5 mM glyphosate.

FIG. 22: Growth of EPSPS, P106S, P106S-P126S and P106S-P126S-K296R in 0 mM and 5 mM glyphosate.

FIG. 23: Specific growth rate of each EPSPS variant in increasing levels of glyphosate. Each heatmap square represents the average of 3 biological replicates.

FIG. 24: Glyphosate and S3P densities in EPSPS X-ray crystallography.

FIG. 25: Crystal structure of ZmEPSPS TIPS (PSKR) with mutations highlighted. The original TIPS mutations are circled (dotted yellow). Complete X-ray crystallography Data Collection and Refinement Statistics are provided in Table 3.

FIG. 26: Location and identity of T102, P106, 1102 and S106 residues in X-ray crystal structure of ZmEPSPS and indicated variants.

FIG. 27: Location and identity of P126, K296, S126 and R296 residues in X-ray crystal structure of ZmEPSPS and indicated variants.

FIG. 28. Panel A) Wild type maize plants were transformed with the indicated variants and with TIPS as a positive control. Plants were regenerated plantlets and on media which included glyphosate to select only those plants that were transformed with the indicated EPSPS variant and demonstrated glyphosate tolerance. Following regeneration of plants, the plants were allowed to recover and were screened for molecular quality and plant health. The resulting advanced plants were moved to the greenhouse for seed production and surplus plants were spray challenged with glyphosate. Panel B) Only two EPSPS variants resulted in enough plants for a glyphosate spray challenge, the TIPS positive control and TIPS-P126S-K296R. Plants were challenged with a glyphosate spray and injury observed 1 week following treatment. Three of the ten TIPS control plants were injured by the glyphosate spray while none of the TIPS-P126S-K296R expressing plants showed visual injury. Panel C) The TIPS plants showed glyphosate injury typical of incomplete tolerance including plant stunting and discolored leaf margins (red arrow) while the TIPS-P126S-K296R plants did not show glyphosate injury.

FIG. 29. Panel A) Outcome of greenhouse spray assay in hybrid background in the following generation, 12 days post treatment, for the non-transgenic control. Panel B) Outcome of greenhouse spray assay in hybrid background in the following generation, 12 days post treatment, for the surviving engineered EPSPS TIPS P126S, M217L, K296R variant. Panel C) Outcome of greenhouse spray assay in hybrid background in the following generation, 12 days post treatment, for the EPSPS TIPS P126S, K296R variant. Panel D) Outcome of greenhouse spray assay in hybrid background in the following generation, 12 days post treatment, for the TIPS positive control. Plants were treated with glyphosate (Roundup PowerMax®3) at the V3 growth stage. Injury ratings were taken at 18 days after treatment, and the panels are shown at 12 days after treatment. The treatments were UTC (untreated control), 1× typical field rate of Roundup (1120 g/ha); and 2× typical field rate of Roundup (2240 g/ha). The plants are shown in pairs (two representative plants for each treatment) with the exception of the UTCs for the experimental samples which are only one plant.

### BRIEF DESCRIPTION OF THE SEQUENCES

SEQ ID NO:1 is the TIPS nucleotide sequence including promoter, transit peptide, exons, introns and 3′ UTR.

SEQ ID NO:2 is the Zea mays EPSPS genomic nucleotide sequence including transit peptide, exons and introns.

SEQ ID NO:3 is the Zea mays EPSPS genomic nucleotide sequence including transit peptide and exons only.

SEQ ID NO:4 is the Zea mays EPSPS genomic nucleotide sequence including exons only.

SEQ ID NO:5 is the Zea mays EPSPS amino acid sequence with the transit peptide.

SEQ ID NO:6 is the Zea mays EPSPS amino acid sequence without the transit peptide.

SEQ ID NO:7 is the Zea mays EPSPS T102I-P106S (TIPS) genomic nucleotide sequence including exons only.

SEQ ID NO:8 is the TIPS amino acid sequence with the transit peptide.

SEQ ID NO:9 is the TIPS amino acid sequence without the transit peptide.

SEQ ID NO:10 is the TIPS-P126S-M217L-K296R amino acid sequence with no transit peptide.

SEQ ID NO:11 is the TIPS-P126S-M217L-K296R amino acid sequence with no transit peptide.

SEQ ID NO:12 is the T102I-P126S-K296R amino acid sequence with no transit peptide.

SEQ ID NO:13 is the P106S-P126S-K296R amino acid sequence with no transit peptide.

SEQ ID NO:14 is the P126S-K296R amino acid sequence with no transit peptide.

SEQ ID NO:15 is the TIPS-P126S amino acid sequence with no transit peptide.

SEQ ID NO:16 is the TIPS-K296R amino acid sequence with no transit peptide.

SEQ ID NO:17 is the TIPS-V66L-M104L amino acid sequence with no transit peptide.

SEQ ID NO:18 is the TIPS-T108S amino acid sequence with no transit peptide.

SEQ ID NO:19 is the TIPS-F150V-H318Y amino acid sequence with no transit peptide.

SEQ ID NO:20 is the TIPS-G116V amino acid sequence with no transit peptide.

SEQ ID NO:21 is the TIPS-1177V amino acid sequence with no transit peptide.

SEQ ID NO:22 is the TIPS-P126L amino acid sequence with no transit peptide.

SEQ ID NO:23 is the P106S-A109V-1163K amino acid sequence with no transit peptide.

### DETAILED DESCRIPTION

5-enolpyruvylshikimate-3-phosphate synthase (EPSPS) catalyzes the reaction of phosphoenolpyruvate (PEP) and shikimate-3-phosphate (S3P) to generate phosphate and 5-enolpyruvylshikimate-3-phosphate (EPSP). This enzyme is well-studied owing to both the central role it plays in aromatic amino acid biosynthesis and its propensity to be inhibited by a common herbicide, glyphosate. Glyphosate's effectiveness against all plant species to inhibit the binding of PEP to EPSPS, its simple synthesis route, and overall low cost has made it the most used herbicide in history. The discovery of a bacterial EPSPS enzyme from Agrobacterium sp. strain CP4 with high glyphosate resistance has enabled transgenic crop designs since the 1990s. Since then, detailed structural and biochemical studies have led to the discovery and rational design of additional glyphosate-resistant mutants. Herbicide tolerant transgenic crops enable better control of weeds and therefore have been widely adopted in many areas, including the United States, where 90% of the soybeans, corn, and cotton grown comprise this trait.

To date, only a small set of mutations are currently known to confer glyphosate resistance to native plant EPSPS and related homologs; and these resistance-conferring mutations also reduce the net activity of this essential enzyme and thus lead to reduced plant growth under native expression. As such, glyphosate insensitivity in class I EPSPS enzymes comes at a high detriment to the catalytic efficiency.

The present disclosure overcomes the limitations known in the art by providing novel, engineered EPSP synthases that exhibit glyphosate resistance and improved catalytic properties (e.g., k_{cat}/Kₘ); the recombinant DNA molecules that encode them, and compositions and methods for using and producing the same. Cells, plants, and seeds expressing engineered EPSP synthases of the present disclosure demonstrate improved glyphosate tolerance and are useful in the methods of agriculture, such as weed control and crop production.

The following definitions and methods are provided to better define the present disclosure and to guide those of ordinary skill in the art in the practice of the present disclosure. Unless otherwise noted, terms are to be understood according to conventional usage by those of ordinary skill in the relevant art.

The present disclosure provides novel, engineered proteins and the recombinant DNA molecules that encode them. As used herein, the term “engineered” refers to a non-natural DNA, protein, cell, or organism that would not normally be found in nature and was created by human intervention. An “engineered protein,” “engineered enzyme,” or “engineered EPSPS,” refers to a protein, enzyme or EPSPS whose amino acid sequence was conceived of and created in the laboratory using one or more of the techniques of biotechnology, protein design, or protein engineering, such as molecular biology, protein biochemistry, bacterial transformation, plant transformation, site-directed mutagenesis, directed evolution using random mutagenesis, genome editing, gene cloning, DNA ligation, DNA synthesis, protein synthesis, and DNA shuffling. For example, an engineered protein may have one or more deletions, insertions, or substitutions relative to the wild-type amino acid sequence of the protein and each deletion, insertion, or substitution may consist of one or more amino acids. For example, genetic engineering can be used to create a DNA molecule encoding an engineered protein, such as an engineered EPSPS that is glyphosate tolerant and comprises at least a first amino acid substitution relative to a wild-type EPSPS protein as described herein.

Examples of engineered proteins provided herein are maize EPSP synthases comprising one or more amino acid substitution(s) chosen from T102I-P106S-P126S-K296R, T102I-P106S-P126S, T102I-P106S-K296R, T102I-P106S-P126S-M217L-K296R, T102I-P106S-V66L-M104L, T102I-P106S-T108S, T102I-P106S-F150V-H318Y, T102I-P106S-P126L, T102I-P106S-G116V, T102I-P106S-1177V, and P106S-A109V-1163K, and combinations thereof, wherein the position of the amino acid substitution(s) is relative to the position of the amino acid sequence provided as SEQ ID NO:6.

In specific embodiments, an engineered protein provided herein comprises one, two, three, four, five, six, seven, cight, nine, ten, or more of any combination of such substitutions. Examples of such combinations include, but are not limited to: T102I-P106S-P126S-K296R, T102I-P106S-P126S, T102I-P106S-K296R, T102I-P106S-P126S-M217L-K296R, T102I-P106S-V66L-M104L, T102I-P106S-T108S, T102I-P106S-F150V-H318Y, T102I-P106S-P126L, T102I-P106S-G116V, T102I-P106S-1177V, and P106S-A109V-1163K.

In one embodiment, engineered proteins provided by the present disclosure are EPSP synthases conferring tolerance to glyphosate. As used herein, “EPSPS” means 5-enolpyruvylshikimate-3-phosphate synthase. EPSPS catalyzes the transfer of the enolpyruvyl moiety of phosphoenolpyruvate (PEP) to the 5-hydroxyl of 3-phosphoshikimate (S3P) to produce phosphate and 5-enolpyruvylshikimate-3-phosphate (EPSP). This reaction is part of the biosynthesis of aromatic amino acids via the shikimate pathway in bacteria, fungi, and plants. Glyphosate is a competitive inhibitor of PEP that when bound to EPSPS inhibits catalysis blocking the shikimate pathway.

Engineered proteins may be produced by changing or modifying a wild-type protein to produce a new protein with modified characteristic(s), for example, a novel combination of useful protein characteristics, such as altered Vₘₐₓ, k_{cat}, Kₘ, Kᵢ, IC₅₀, substrate specificity, inhibitor/herbicide specificity, substrate selectivity, the ability to interact with other components in the cell such as partner proteins or membranes, and protein stability, among others.

Changes may be made at a specific amino acid position in a protein by substituting an alternate amino acid for the amino acid found in that position in the wild-type protein sequence. As used herein, the term “substitution” or “substituting” refers to replacing one amino acid with another amino acid. A substitution is indicated in standard scientific nomenclature by X #Y (where X is the original or wild-type amino acid, #is the amino acid position in the protein's amino acid sequence, and Y is the amino acid to be substituted for X). DNA sequences encoding EPSP synthases with the amino acid substitution(s) described herein can be produced by introducing changes into the DNA sequence encoding the EPSPS using methods known in the art and the information provided in Table 1.

TABLE 1
Second Base in Codon

| | | U | C | A | G | | |
|---|---|---|---|---|---|---|---|
| First Base in Codon | U | 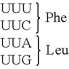 | 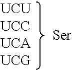 | 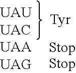 | 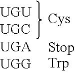 | U C A G | Third Base in Codon |
| | C | 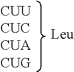 | 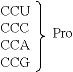 | 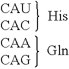 | 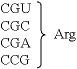 | U C A G | |
| | A | 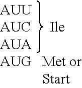 | 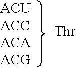 | 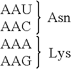 | 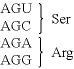 | U C A G | |
| | G | 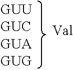 | 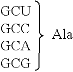 | 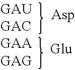 | 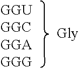 | G | |

EPSPS sequences from different plant species can be aligned and compared with SEQ ID NO: 6 using standard bioinformatic and sequence analysis tools, such as the Clustal software tools or implementations of the Needleman-Wunsch or the Smith-Waterman algorithms. The substitution(s) provided herein thus can be made in any plant EPSP synthases by aligning the amino acid sequence of the target plant EPSPS with SEQ ID NO:6, identifying the equivalent position of the amino acid for the desired substitution in the target EPSPS sequence relative to the specific amino acid position set forth in SEQ ID NO:6, and making the substitution using the methods provided herein.

Amino acid changes may be made as a single amino acid substitution in the protein or in combination with one or more other change(s) or mutation(s), such as one or more other amino acid substitution(s), deletion(s), or addition(s). A protein can be changed or modified by one or more substitutions that are made to the amino acid sequence relative to a reference sequence, such as the wild-type sequence, by changing the DNA sequence encoding the protein. Changes or modifications may be made by any method known to those of skill in the art. In one embodiment, the present disclosure therefore provides an engineered protein, such as an EPSPS having one or more amino acid substitution(s) chosen from T102I-P106S-P126S-K296R, T102I-P106S-P126S, T102I-P106S-K296R, T102I-P106S-P126S-M217L-K296R, T102I-P106S-V66L-M104L, T102I-P106S-T108S, T102I-P106S-F150V-H318Y, T102I-P106S-P126L, T102I-P106S-G116V, T102I-P106S-1177V, and P106S-A109V-1163K, and combinations thereof, wherein the position of the amino acid substitution(s) is relative to the position of the amino acid sequence provided as SEQ ID NO: 6. In specific embodiments, an engineered protein provided herein comprises one, two, three, four, five, six, seven, eight, nine, ten, or more of any combination of such substitutions.

In another embodiment, the present disclosure provides an engineered protein comprising one or more amino acid substitution(s) described herein, and the recombinant DNA molecules encoding it, having at least about 85% sequence identity, about 90% sequence identity, about 91% sequence identity, about 92% sequence identity, about 93% sequence identity, about 94% sequence identity, about 95% sequence identity, about 96% sequence identity, about 97% sequence identity, about 98% sequence identity, about 99% sequence identity, about 99.5% sequence identity, about 99.8% sequence identity and about 99.9% sequence identity to SEQ ID NO: 6.

Engineered proteins provided by the present disclosure thus, in certain embodiments, provide an engineered EPSPS with one or more altered protein characteristics relative to a similar EPSPS, or wild-type EPSPS, found in nature. In one embodiment of the present disclosure, such altered protein characteristics may include those that result in decreased sensitivity, or increased tolerance, to glyphosate or improved enzyme kinetics, as compared to a similar wild-type EPSPS, for instance an EPSPS comprising the sequence of SEQ ID NO:6.

Such EPSPS variants or engineered EPSP synthases that exhibit a decreased affinity for glyphosate while simultaneously maintaining the catalytic efficiency of the enzyme therefore provide a method of achieving glyphosate tolerance in crops. EPSPS variants or engineered EPSP synthases can be evaluated by measuring the enzyme's maximal velocity (Vₘₐₓ), representing how fast the enzyme can catalyze the reaction under substrate saturation conditions, the turnover number (k_{cat}), the Michaelis-Menten Constant (Kₘ), representing the substrate concentration at half the enzyme's catalytic capacity, and the second order rate constant (k_{cat}/Kₘ). The high proportion of carbon flux through the shikimate pathway requires a highly efficient EPSPS (maximum catalytic efficiency) to prevent metabolic limitations or bottlenecks as required by a wide variety of growth conditions in various developmental stages.

As used herein, the term “recombinant” refers to a non-naturally occurring DNA, protein, cell, seed, or organism that is the result of genetic engineering or genome editing and as such would not normally be found in nature and was created by human intervention. A “recombinant DNA molecule” is a DNA molecule comprising a DNA sequence that is the result of human intervention, for example, a DNA molecule that is engineered or a DNA molecule that encodes an engineered protein or engineered enzyme. Another example is a DNA molecule comprised of a combination of at least two DNA molecules heterologous to each other, such as a protein-coding DNA molecule and an operably linked heterologous promoter. Another example is a DNA molecule encoding an EPSPS protein comprising any one or more of the amino acid substitutions described herein. A “recombinant protein” is a protein comprising an amino acid sequence that is the result of human intervention, for example, an engineered protein. A recombinant cell, seed, or organism is a cell, seed, or organism comprising a modified genome, created as a result of the use of genome editing techniques or the use of plant transformation techniques, for example a plant cell, seed, plant, or plant part comprising a DNA molecule or protein of the present disclosure.

As used herein, “wild-type” means a naturally occurring or typically occurring form. A “wild-type DNA molecule” or “wild-type protein” is the version of a DNA molecule or protein that is naturally or typically occurring. For crop plants, this would be the version of a DNA molecule or protein that is typically found in that crop. The DNA sequence or amino acid sequence of the wild-type DNA molecule or protein is the sequence that typically exists in that crop. A wild-type version of a DNA molecule or protein may be useful as a reference DNA molecule or reference protein for comparison with a recombinant or engineered DNA molecule or protein. An example of a wild-type protein useful for comparison with the engineered proteins provided by the present disclosure is the EPSPS from maize provided as SEQ ID NO:6. Other wild-type EPSP synthases useful for comparison with the engineered proteins provided by the present disclosure are known from other plants.

A “wild-type plant” is a naturally occurring plant. Such wild-type plants may also be useful for comparison with a plant comprising a recombinant or engineered DNA molecule or protein. An example of a wild-type plant useful for comparison with plants comprising a recombinant or engineered DNA molecule or protein may be a plant of the same type as the plant comprising the engineered DNA molecule or protein, such as a protein conferring an herbicide tolerance trait, and as such is genetically distinct from the plant comprising the herbicide tolerance trait. An example of a wild-type plant useful for comparison for maize plants includes glyphosate-sensitive LH244 maize (ATCC deposit number PTA-1173, ATCC®, Manassas, Virginia USA).

In certain embodiments, wild-type plants may also be used or referred to as “control plants.” As used herein, “control” means an experimental control designed for comparison purposes. For example, a control plant is a plant of the same type as the experimental plant (that is, the plant to be tested) but does not contain the transgenic insert, recombinant DNA molecule, or genome modification of the experimental plant.

As used herein, the term “DNA” or “DNA molecule” refers to a double-stranded DNA molecule of genomic or synthetic origin (that is, a polymer of deoxyribonucleotide bases or a polynucleotide molecule) read from the 5′ (upstream) end to the 3′ (downstream) end. As used herein, the term “DNA sequence” refers to the nucleotide sequence of a DNA molecule. The nomenclature used herein corresponds to that of by Title 37 of the United States Code of Federal Regulations § 1.822, and set forth in the tables in WIPO Standard ST.25 (1998), Appendix 2, Tables 1 and 3.

The present disclosure provides a nucleic acid molecule encoding a maize EPSPS having one or more amino acid substitution(s) chosen from T102I-P106S-P126S-K296R, T102I-P106S-P126S, T102I-P106S-K296R, T102I-P106S-P126S-M217L-K296R, T102I-P106S-V66L-M104L, T102I-P106S-T108S, T102I-P106S-F150V-H318Y, T102I-P106S-P126L, T102I-P106S-G116V, T102I-P106S-1177V, and P106S-A109V-1163K, and combinations thereof, wherein the position of the amino acid substitution(s) is relative to the position of the amino acid sequence provided as SEQ ID NO:6.

As used herein, the term “protein-coding DNA molecule” refers to a DNA molecule comprising a DNA sequence that encodes a protein. A DNA sequence that encodes a protein (also known as a “protein-coding sequence”) is composed of a series of three-nucleotide sequences called codons, which serve as the genetic information that is used to produce the amino acid sequence of protein. As used herein, the term “protein” refers to a chain of amino acids linked by peptide (amide) bonds and includes both polypeptide chains that are folded or arranged in a biologically functional way and polypeptide chains that are not. As used herein, a “sequence” means a sequential arrangement of nucleotides or amino acids. The boundaries of a protein-coding sequence are usually determined by a translation start codon at the 5′-terminus and a translation stop codon at the 3′-terminus.

As used herein, the term “isolated” refers to at least partially separating a molecule from other molecules typically associated with it in its natural state. In one embodiment, the term “isolated” refers to a DNA molecule that is separated from the nucleic acids that normally flank the DNA molecule in its natural state. For example, a DNA molecule encoding a protein that is naturally present in a bacterium would be an isolated DNA molecule if it was not within the DNA of the bacterium from which the DNA molecule encoding the protein is naturally found. Thus, a DNA molecule fused to or operably linked to one or more other DNA molecule(s) with which it would not be associated in nature, for example as the result of recombinant DNA or plant transformation techniques, is considered isolated herein. Such molecules are considered isolated even when integrated into the chromosome of a host cell or present in a nucleic acid solution with other DNA molecules.

Any number of methods well known to those skilled in the art can be used to isolate and manipulate a DNA molecule, or fragment thereof, as disclosed herein. For example, polymerase chain reaction (PCR) technology can be used to amplify a particular starting DNA molecule and/or to produce variants of the original molecule. DNA molecules, or fragment thereof, can also be obtained by other techniques, such as by directly synthesizing the fragment by chemical means, as is commonly practiced by using an automated oligonucleotide synthesizer.

Because of the degeneracy of the genetic code, a different DNA sequences can encode the same amino acid sequence. For example, Table 1 provides the universal genetic code chart showing all possible mRNA triplet codons (where T in the DNA molecule is replaced by U in the RNA molecule) and the amino acid encoded by each codon. DNA sequences encoding EPSPS with the amino acid substitutions described herein can be produced by introducing changes or mutations into the DNA sequence encoding wild-type EPSPS using methods known in the art and the information provided in Table 1. It is well within the capability of one of skill in the art in view of the present disclosure to create alternative DNA sequences encoding engineered proteins as described herein. These variant or alternative DNA sequences are within the scope of the embodiments described herein. As used herein, references to “essentially the same” sequence refers to sequences which encode amino acid substitutions, deletions, additions, or insertions that do not materially alter the functional activity of the protein encoded by the DNA molecule of the embodiments described herein. Allelic variants of the nucleotide sequences encoding a wild-type or engineered protein are also encompassed within the scope of the embodiments described herein. Substitution of amino acids other than those specifically exemplified or naturally present in a wild-type or engineered EPSPS are also contemplated within the scope of the embodiments described herein, so long as the EPSPS having the substitution still retains substantially the same functional activity described herein.

As used herein, the term “percent sequence identity” or “% sequence identity” refers to the percentage of identical nucleotides or amino acids in a linear polynucleotide or amino acid sequence of a reference (“query”) sequence (or its complementary strand) as compared to a test (“subject”) sequence (or its complementary strand) when the two sequences are optimally aligned (with appropriate nucleotide or amino acid insertions, deletions, or gaps totaling less than 20 percent of the reference sequence over the window of comparison). Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and by computerized implementations of these algorithms such as GAP, BESTFIT, FASTA, and TFASTA available as part of the Sequence Analysis software package of the GCG® Wisconsin Package® (Accelrys Inc., San Diego, CA), MEGAlign (DNAStar Inc., 1228 S. Park St., Madison, WI 53715), and MUSCLE (version 3.6) (RC Edgar, “MUSCLE: multiple sequence alignment with high accuracy and high throughput” Nucleic Acids Research 32 (5): 1792-7 (2004)) for instance with default parameters. An “identity fraction” for aligned segments of a test sequence and a reference sequence is the number of identical components that are shared by the two aligned sequences divided by the total number of components in the portion of the reference sequence segment being aligned, that is, the entire reference sequence or a smaller defined part of the reference sequence. Percent sequence identity is represented as the identity fraction multiplied by 100. The comparison of one or more sequences may be to a full-length sequence or a portion thereof, or to a longer sequence.

As used herein, a “DNA construct” is a recombinant DNA molecule comprising two or more heterologous DNA sequences. DNA constructs are useful for transgene expression and may be comprised in vectors and plasmids. DNA constructs may be used in vectors for transformation, that is the introduction of heterologous DNA into a host cell, to produce transgenic plants and cells, and as such may also be contained in the plastid DNA or genomic DNA of a transgenic plant, secd, cell, or plant part. As used herein, a “vector” means any recombinant DNA molecule that may be used for the purpose of bacterial or plant transformation. DNA molecules provided herein can, for example, be inserted into a vector as part of a construct having the DNA molecule operably linked to a gene expression element that functions in a plant to affect expression of the protein encoded by the DNA molecule. Methods for constructing DNA constructs and vectors are well known in the art and described in detail in, for example, handbooks and laboratory manuals including M. R. Green and J. Sambrook, “Molecular Cloning: A Laboratory Manual” (Fourth Edition) ISBN: 978-1-936113-42-2, Cold Spring Harbor Laboratory Press, NY (2012). The components for a DNA construct, or a vector comprising a DNA construct, include one or more gene expression elements operably linked to a transcribable DNA sequence, such as the following: a promoter for the expression of an operably linked DNA, an operably linked protein-coding DNA molecule, and an operably linked 3′ untranslated region (UTR). Gene expression elements useful in practicing the present disclosure include, but are not limited to, one or more of the following type of elements: promoter, 5′ UTR, enhancer, leader, cis-acting element, intron, targeting or transit sequence, 3′ UTR, and one or more selectable marker transgenes.

The term “transgene” refers to a DNA molecule artificially incorporated into the genome of an organism as a result of human intervention, such as by plant transformation methods. As used herein, the term “transgenic” means comprising a transgene, for example a “transgenic plant” refers to a plant comprising a transgene in its genome and a “transgenic trait” refers to a characteristic or phenotype conveyed or conferred by the presence of a transgene incorporated into the plant genome. As a result, the transgenic plant is something distinctly different from the related wild-type plant and the transgenic trait is a trait not naturally found in the wild-type plant. Transgenic plants of the present disclosure comprise the recombinant DNA molecules and engineered proteins provided by the present disclosure.

As used herein, the term “heterologous” refers to the relationship between two or more things not normally associated in nature, for instance that are derived from different sources or not normally found in nature together in any other manner. For example, a DNA molecule or protein may be heterologous with respect to another DNA molecule, protein, cell, plant, seed, or organism if not normally found in nature together or in the same context. In certain embodiments, a first DNA molecule is heterologous to a second DNA molecule if the two DNA molecules are not normally found in nature together in the same context. For instance, a protein-coding recombinant DNA molecule is heterologous with respect to an operably linked promoter if such a combination is not normally found in nature. Similarly, a protein is heterologous with respect to a second operably linked protein, such as a transit peptide, if such combination is not normally found in nature. In another embodiment, a recombinant DNA molecule encoding an EPSPS is heterologous with respect to an operably linked promoter that is functional in a plant cell if such combination is not normally found in nature. A recombinant DNA molecule also may be heterologous with respect to a cell, seed, or organism into which it is inserted when it would not naturally occur in that cell, seed, or organism.

A “heterologous protein” is a protein present in a plant, seed, cell, tissue, or organism in which it does not naturally occur or operably linked to a protein with which it is not naturally linked. An example of a heterologous protein is an engineered EPSPS protein comprising at least a first amino acid substitution described herein that is expressed in any plant, seed, cell, tissue, or organism. Another example is a protein operably linked to a second protein, such as a transit peptide or herbicide-tolerant protein, with which it is not naturally linked, or a protein introduced into a plant cell in which it does not naturally occur using the techniques of genetic engineering.

As used herein, “operably linked” means two or more DNA molecules or two or more proteins linked in manner so that one may affect the function of the other. Operably linked DNA molecules or operably linked proteins may be part of a single contiguous molecule and may or may not be adjacent. For example, a promoter is operably linked with a protein-coding DNA molecule in a DNA construct where the two DNA molecules are so arranged that the promoter may affect the expression of the transgene.

The DNA constructs of the present disclosure may include a promoter operably linked to a protein-coding DNA molecule provided by the present disclosure, whereby the promoter drives expression of the recombinant protein molecule. Promoters useful in practicing the present disclosure include those that function in a cell for expression of an operably linked polynucleotide, such as a bacterial or plant promoter. Plant promoters are varied and well known in the art and include, for instance, those that are inducible, viral, synthetic, constitutive, temporally regulated, spatially regulated, and/or spatio-temporally regulated.

In one embodiment of the present disclosure, a DNA construct provided herein includes a DNA sequence encoding a targeting sequence that is operably linked to a heterologous DNA sequence encoding a maize EPSPS, whereby the targeting sequence facilitates localizing the polypeptide molecule within the cell. Targeting sequences are known in the art as signal sequences, targeting peptides, localization sequences, and transit peptides. An example of a targeting sequence is a chloroplast transit peptide (CTP), a mitochondrial targeting sequence (MTS), or a dual chloroplast and mitochondrial targeting or transit peptide. By facilitating protein localization within the cell, the targeting sequence may increase the accumulation of recombinant protein, protect the protein from proteolytic degradation, and/or enhance the level of herbicide tolerance, and thereby reduce levels of injury in the cell, seed, or organism after herbicide application. CTPs and other targeting molecules that may be used in connection with the present disclosure are well known in the art.

As used herein, “expression”, “expressing”, “protein expression”, and “expressing a protein” mean the production of a protein through the process of transcribing a DNA molecule into messenger RNA (mRNA) and translating the mRNA into polypeptide chains, which are ultimately folded into proteins. A protein-coding DNA molecule may be operably linked to a heterologous promoter in a DNA construct for use in expressing the protein in a cell transformed with the recombinant DNA molecule.

In one aspect the present disclosure provides cells, tissues, plants, and seeds comprising the recombinant DNA molecules or expressing the engineered proteins, such as the engineered EPSP synthases, of the present disclosure. These cells, tissues, plants, and seeds comprising the recombinant DNA molecules or engineered proteins exhibit tolerance to glyphosate.

One method of producing such cells, tissues, plants, and seeds is through plant transformation. Suitable methods for transformation of host plant cells for use with the current disclosure include any method by which DNA can be introduced into a cell (for example, where a recombinant DNA construct is stably integrated into a plant chromosome) and are well known in the art. Two effective, and widely utilized, methods for cell transformation are Agrobacterium-mediated transformation and microprojectile bombardment-mediated transformation. Microprojectile bombardment methods are illustrated, for example, in U.S. Pat. Nos. 5,550,318; 5,538,880; 6,160,208; and 6,399,861. Agrobacterium-mediated transformation methods are described, for example in U.S. Pat. No. 5,591,616, which is incorporated herein by reference in its entirety.

Another method of producing such cells, tissues, plants, and seeds is through genome cditing. As used herein, the term “genome editing” refers to the use of genome editing methods and a site-specific genome modification enzyme to modify a nucleotide sequence. Suitable methods for altering a wild-type DNA sequence at a pre-determined chromosomal site include any method known in the art. Exemplary methods include the use of sequence specific nucleases, such as zinc-finger nucleases, engineered or native meganucleases, TALE-endonucleases, or an RNA-guided endonuclease (for example, a Clustered Regularly Interspersed Short Palindromic Repeat (CRISPR)/Cas9 system, a CRISPR/Cpf1 system, a CRISPR/CasX system, a CRISPR/CasY system, or a CRISPR/Cascade system). Several embodiments relate to methods of genome editing by using single-stranded oligonucleotides to introduce precise base pair modifications in a plant genome, as described by Sauer et al., Plant Physiology 170 (4): 1917-1928 (2016). Methods of genome editing to modify, delete, or insert nucleic acid sequences into genomic DNA are known in the art.

As used herein, “modified” in the context of a plant, plant seed, plant part, plant cell, and/or plant genome, refers to a plant, plant seed, plant part, plant cell, and/or plant genome comprising an engineered change in the expression level and/or endogenous sequence of one or more genes of interest relative to a wild-type or control plant, plant seed, plant part, plant cell, and/or plant genome. Indeed, the term “modified” may further refer to a plant, plant seed, plant part, plant cell, and/or plant genome having one or more amino acid substitutions affecting an endogenous EPSPS gene introduced through chemical mutagenesis, transposon insertion or excision, or any other known mutagenesis technique, or introduced through genome editing. In an aspect, a modified plant, plant seed, plant part, plant cell, and/or plant genome can comprise one or more transgenes. For clarity, therefore, a modified plant, plant seed, plant part, plant cell, and/or plant genome includes a mutated, edited and/or transgenic plant, plant seed, plant part, plant cell, and/or plant genome having a modified sequence of an EPSPS gene relative to a wild-type or control plant, plant seed, plant part, plant cell, and/or plant genome. Furthermore, the modification may alter the activity of the protein encoded by the EPSPS gene as compared to the activity of the protein encoded by the EPSPS gene in an otherwise identical plant.

Modified plants, plant parts, seeds, etc., may have been subjected to mutagenesis, genome editing or site-directed integration, genetic transformation, or a combination thereof. Such “modified” plants, plant seeds, plant parts, and plant cells include plants, plant seeds, plant parts, and plant cells that are offspring or derived from “modified” plants, plant seeds, plant parts, and plant cells that retain the molecular change (e.g., change in expression level and/or activity in the presence of glyphosate) to the EPSPS gene. A modified seed provided herein may give rise to a modified plant provided herein. A modified plant, plant seed, plant part, plant cell, or plant genome provided herein may comprise a recombinant DNA construct or vector or genome edit as provided herein.

Several embodiments relate to a plant comprising in its genome a modified EPSPS coding sequence, wherein the modified EPSPS coding sequence encodes a glyphosate-tolerant EPSPS as described herein. In certain embodiments, genome editing methods are utilized for the modification or replacement of an existing coding sequence, such as an EPSPS coding sequence, within a plant genome with a sequence encoding an engineered protein, such as an engineered EPSPS coding sequence of the present disclosure. In some embodiments, the native EPSPS coding sequence is modified to comprise one or more targeted nucleotide changes, additions, deletions, or other modifications, such that the modified EPSPS coding sequence encodes a glyphosate-tolerant EPSPS that comprises an amino acid substitution combination selected from the group consisting of: T102I-P106S-P126S-K296R, T102I-P106S-P126S, T102I-P106S-K296R, T102I-P106S-P126S-M217L-K296R, T102I-P106S-V66L-M104L, T102I-P106S-T108S, T102I-P106S-F150V-H318Y, T102I-P106S-P126L, T102I-P106S-G116V, T102I-P106S-1177V, and P106S-A109V-I163K, and combinations thereof, wherein the position of the amino acid substitution(s) is relative to the position of the amino acid sequence provided as SEQ ID NO:6.

Several embodiments relate to the use of known genome editing methods, and a site-specific genome modification enzyme, such as zinc-finger nucleases, engineered or native meganucleases, TALE-endonucleases, or an RNA-guided endonuclease (for example, a Clustered Regularly Interspersed Short Palindromic Repeat (CRISPR)/Cas9 system, a CRISPR/Cpf1 system, a CRISPR/CasX system, a CRISPR/CasY system, a CRISPR/Cascade system) to modify or replace an existing EPSPS coding sequence in the genome of a plant. Several embodiments therefore relate to providing a site-specific genome modification enzyme capable of recognizing a specific nucleotide sequence of interest, such as a maize EPSPS sequence, within a genome of a plant to allow for alteration of the EPSPS sequence by non-templated editing or by templated editing.

Several embodiments relate to a recombinant DNA construct comprising an expression cassette(s) encoding a site-specific nuclease and/or any associated protein(s) to carry out genomc modification. These nuclease-expressing cassette(s) may be present in the same molecule or vector as a donor template for templated editing wherein the donor template encodes a glyphosate-tolerant maize EPSPS protein as described herein in cis or on a separate molecule or vector (in trans). Several methods for templated editing are known in the art involving different sequence-specific nucleases (or complexes of proteins and/or guide RNA) that cut the genomic DNA to produce a double strand break (DSB) or nick at a desired genomic site or locus. As understood in the art, during the process of repairing the DSB or nick introduced by the nuclease enzyme, the donor template DNA may become integrated into the genome at the site of the DSB or nick.

As used herein, the term “site-specific genome modification enzyme” refers to any enzyme that can modify a nucleotide sequence in a sequence-specific manner. In some embodiments, a site-specific genome modification enzyme modifies the genome by inducing a single-strand break. In some embodiments, a site-specific genome modification enzyme modifies the genome by inducing a double-strand break. In some embodiments, a site-specific genome modification enzyme comprises a cytidine deaminase. In some embodiments, a site-specific genome modification enzyme comprises an adenine deaminase. In the present disclosure, site-specific genome modification enzymes include endonucleases, recombinases, transposases, deaminases, helicases and any combination thereof. In some embodiments, the site-specific genome modification enzyme is a sequence-specific nuclease.

In one aspect, the site-specific genome modification enzyme comprises an endonuclease selected from a meganuclease, a zinc-finger nuclease (ZFN), a transcription activator-like effector nucleases (TALEN), an Argonaute (non-limiting examples of Argonaute proteins include Thermus thermophilus Argonaute (TtAgo), Pyrococcus furiosus Argonaute (PfAgo), Natronobacterium gregoryi Argonaute (NgAgo), an RNA-guided nuclease, such as a CRISPR associated nuclease (non-limiting examples of CRISPR associated nucleases include Cas1, Cas1B, Cas2, Cas3, Cas4, Cas5, Cas6, Cas7, Cas8, Cas9 (also known as Csn1 and Csx12), Cas10, Cas12a (also known as Cpf1), Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, CasX, CasY, homologs thereof, or modified versions thereof).

In some embodiments, the site-specific genome modification enzyme comprises a DNA binding domain operably linked to a deaminase. In some embodiments, the site-specific genome modification enzyme further comprises uracil DNA glycosylase (UGI). In some embodiments, the deaminase is a cytidine deaminase. In some embodiments, the deaminase is an adenine deaminase. In some embodiments, the deaminase is an APOBEC deaminase. In some embodiments, the deaminase is an activation-induced cytidine deaminase (AID). In some embodiments, the DNA binding domain is a zinc-finger DNA-binding domain, a TALE DNA-binding domain, a Cas9 nuclease, a Cas12a nuclease, a catalytically inactive Cas9 nuclease, a catalytically inactive Cas12a nuclease, a Cas9 nickase, or a Cpf1 nickase.

In some embodiments, the site-specific genome modification enzyme is a recombinase. Non-limiting examples of recombinases include a tyrosine recombinase attached to a DNA recognition motif provided herein is selected from the group consisting of a Cre recombinase, a Gin recombinase, a F1p recombinase, and a Tnp1 recombinase. In an aspect, a Cre recombinase or a Gin recombinase provided herein is tethered to a zinc-finger DNA-binding domain, or a TALE DNA-binding domain, or a Cas9 nuclease. In another aspect, a serine recombinase attached to a DNA recognition motif provided herein is selected from the group consisting of a PhiC31 integrase, an R4 integrase, and a TP-901 integrase. In another aspect, a DNA transposase attached to a DNA binding domain provided herein is selected from the group consisting of a TALE-piggyBac and TALE-Mutator.

In one aspect, the present disclosure provides cells, plants, and seeds that are tolerant to glyphosate. Such cells, plants, and seeds are useful in the methods of agriculture, such as weed control and crop production.

As used herein, “herbicide” is any molecule that is used to control, prevent, or interfere with the growth of one or more plants. Exemplary herbicides include 5-enolpyruvylshikimate-3-phosphate synthase (EPSPS) inhibitors (for example glyphosate), acetyl-CoA carboxylase (ACCase) inhibitors (for example aryloxyphenoxy propionates and cyclohexanediones), acetolactate synthase (ALS) inhibitors (for example sulfonylureas, imidazolinones, triazolopyrimidines, and triazolinones), synthetic auxins (for example phenoxys, benzoic acids, carboxylic acids, semicarbazones), photosynthesis (photosystem II) inhibitors (for example triazines, triazinones, nitriles, benzothiadiazoles, and ureas), glutamine synthetase (GS) inhibitors (for example glufosinate and bialaphos), 4-hydroxyphenylpyruvate dioxygenase (HPPD) inhibitors (for example isoxazoles, pyrazolones, and triketones), protoporphyrinogen oxidase (PPO) inhibitors (for example diphenylethers, N-phenylphthalimide, aryl triazinones, and pyrimidinediones), very long-chain fatty acid inhibitors (for example chloroacetamides, oxyacetamides, and pyrazoles), cellulose biosynthesis inhibitors (for example indaziflam), photosystem I inhibitors (for example paraquat), microtubule assembly inhibitors (for example pendimethalin), and phytoene desaturase (PDS) inhibitors (for example norflurazone), among others.

As used herein, “glyphosate tolerance” or “glyphosate-tolerant” with respect to a protein means the ability to maintain at least some of its activity or function in the presence of glyphosate. For example, an EPSPS is glyphosate-tolerant if it maintains at least some of its enzymatic activity in the presence of glyphosate. Glyphosate tolerance can be measured by any means known in the art. For example, the enzymatic activity of an EPSPS can be measured by a bacterial assay, such as the growth assays described herein, whereby a recombinant EPSPS is expressed in a bacterial cell otherwise lacking EPSPS activity and the ability of the recombinant EPSPS to complement this knockout phenotype is measured. In another example, enzymatic activity of an EPSPS can be measured by analyzing enzyme kinetics in the presence and absence of glyphosate. Glyphosate tolerance may be complete or partial insensitivity to glyphosate.

As used herein, “glyphosate tolerance” or “glyphosate-tolerant” with respect to an organism, plant, seed, tissue, part, or cell means the organism, plant, seed, tissue, part, or cell's ability to resist the toxic effects of glyphosate when applied. For example, a glyphosate-tolerant plant can survive or continue to grow in the presence of glyphosate. The glyphosate tolerance of a plant, seed, plant tissue, plant part, or cell may be measured by comparing the plant, seed, plant tissue, plant part, or cell to a suitable control. For example, the glyphosate tolerance may be measured by applying glyphosate to a plant comprising a recombinant DNA molecule encoding a modified EPSPS capable of conferring glyphosate tolerance (the test plant) and a plant not comprising the recombinant DNA molecule encoding the modified EPSPS capable of conferring glyphosate tolerance (the control plant) and subsequently comparing the injury rates of the two plants. Glyphosate tolerance of the test plant is indicated by a decreased injury rate when compared to the injury rate of the control plant. A glyphosate-tolerant plant, seed, plant tissue, plant part, or cell exhibits a decreased response to the toxic effects of glyphosate when compared to a control plant, seed, plant tissue, plant part, or cell.

As used herein, a “glyphosate tolerance trait” is a trait imparting improved glyphosate tolerance to a plant as compared to the wild-type plant. Contemplated plants that may be produced with the glyphosate tolerance trait of the present disclosure could include, for instance, any plant including monocot and dicot crop plants, among others. Examples of monocot crop plants that may be produced with the glyphosate tolerance trait of the present disclosure include, but are not limited to, Zea mays, Sorghum bicolor, Triticum aestivum, Secale cereale, Musa paradisiaca L., Musa sapientum L., Allium sativum, Allium ampeloprasum, Allium cepa L., Oryza sativa, Asparagus officinalis, Avena sativa L., and Hordeum vulgare. Examples of dicot crop plants that may be produced with the glyphosate tolerance trait of the present disclosure include, but are not limited to, Glycine max, Gossypium hirsutum, Goyssypium barbadense, Brassica napus, and Brassica rapa.

A maize plant, as referenced herein, refers to any plant selected from the genus Zea, including, but not limited to, any plant selected from the species Zea mays L.

As used herein, a “weed” is any undesired plant. A plant may be considered generally undesirable for agriculture or horticulture purposes (for example, Amaranthus species) or may be considered undesirable in a particular situation (for example, a crop plant of one species in a field of a different species, also known as a volunteer plant). Weeds are commonly known in the art and vary by geography, season, growing environment, and time. Lists of weed species are available from agricultural and scientific societies (such as the Weed Science Society of America and the Canadian Weed Science Society), government agencies (such as the United States Department of Agriculture and the Australia Department of the Environment and Energy), and industry and farmer associations (such as the United Soybean Board, the National Corn Growers Association, and the Canola Council of Canada).

The herbicide application may be the recommended commercial rate (1×) or any fraction or multiple thereof, such as twice the recommended commercial rate (2×). In certain embodiments, herbicide rates may be expressed as grams per hectare (g/h) or pounds per acre (lbs/acre), acid equivalent per pound per acre (lb ae/acre), acid equivalent per gram per hectare (g ae/ha), pounds active ingredient per acre (lb ai/acre), or grams active ingredient per hectare (g ai/ha) depending on the herbicide and the formulation. The plant growth area may or may not comprise weed plants at the time of herbicide application. An herbicidally-effective dose of glyphosate for use in an area for controlling weeds should consist of a range from about 0.1× to about 3× label rate(s) over a growing season. One (1) acre is equivalent to 2.47105 hectares and one (1) pound is equivalent to 453.592 grams. Herbicide rates can be converted between English and metric as: (lb ai/ac) multiplied by 1.12=(kg ai/ha) and (kg ai/ha) multiplied by 0.89=(lb ai/ac).

Herbicide applications may be sequential or tank mixed with one, two, or a combination of several herbicides or any other compatible herbicide. Multiple applications of one herbicide or of two or more herbicides, in combination or alone, may be used over a growing season to areas comprising plants of the present disclosure for the control of a broad spectrum of dicot weeds, monocot weeds, or both, for example, two applications (such as a pre-planting application and a post-emergence application or a pre-emergence application and a post-emergence application) or three applications (such as a pre-planting application, a pre-emergence application, and a post-emergence application or a pre-emergence application and two post-emergence applications).

Accordingly, the current disclosure provides methods for selectively controlling weeds in a field containing a crop that involve planting the field with crop seeds or plants which are glyphosate tolerant as a result of being transformed with a recombinant DNA molecule encoding an EPSPS disclosed herein or an active variant or fragment thereof, or as a result of being modified to comprise the site-specific EPSPS gene modifications disclosed herein, and applying to the crop and weeds in the field a sufficient amount of glyphosate to control the weeds without significantly affecting the crop.

The plants, progeny, seeds, plant cells, and plant parts of the present disclosure may also contain one or more additional traits. Additional traits may be introduced by crossing a plant comprising the recombinant DNA molecules provided by the present disclosure with another plant containing one or more additional trait(s). As used herein, “crossing” means breeding two individual plants to produce a progeny plant. Two plants may be crossed to produce progeny that contain the desirable traits from each parent. As used herein “progeny” means the offspring of any generation of a parent plant, and progeny comprise an herbicide-tolerance trait provided by the present disclosure and inherited from at least one parent plant. Additional trait(s) also may be introduced by any means known in the art. Such additional traits include, but are not limited to, increased insect resistance, increased water use efficiency, increased yield performance, increased drought resistance, increased seed quality, improved nutritional quality, hybrid seed production, and herbicide-tolerance, in which the trait is measured with respect to a wild-type plant. Exemplary additional herbicide tolerance traits may include transgenic or non-transgenic tolerance to one or more herbicides such as ACCase inhibitors (for example, aryloxyphenoxy propionates and cyclohexanediones), ALS inhibitors (for example, sulfonylureas, imidazolinones, triazolopyrimidines, and triazolinones) EPSPS inhibitors (for example, glyphosate), synthetic auxins (for example, phenoxys, benzoic acids, carboxylic acids, semicarbazones), photosynthesis inhibitors (for example, triazines, triazinones, nitriles, benzothiadiazoles, and ureas), glutamine synthesis inhibitors (for example, glufosinate), HPPD inhibitors (for example, isoxazoles, pyrazolones, and triketones), PPO inhibitors (for example, diphenylethers, N-phenylphthalimide, aryl triazinones, and pyrimidinediones), and long-chain fatty acid inhibitors (for example, chloroacetamindes, oxyacetamides, and pyrazoles), among others. Exemplary insect resistance traits may include resistance to one or more insect members within one or more of the orders of Lepidoptera, Coleoptera, Hemiptera, Thysanoptera, Diptera, Hymenoptera, and Orthoptera, among others. Such additional traits are known to one of skill in the art in light of the present disclosure; for example, and a list of such transgenic traits is provided by the United States Department of Agriculture's (USDA) Animal and Plant Health Inspection Service (APHIS).

Plants and progeny that are glyphosate tolerant may be used with any breeding methods that are commonly known in the art. In plant lines comprising two or more traits, the traits may be independently segregating, linked, or a combination of both in plant lines comprising three or more traits. Backcrossing to a parental plant and outcrossing with a non-traited plant are also contemplated, as is vegetative propagation. Descriptions of breeding methods that are commonly used for different traits and crops are well-known to those of skill in the art. To confirm the presence of the transgene(s) or a genome modification in a plant or seed, a variety of assays may be performed. Such assays include, for example, molecular biology assays, such as Southern and northern blotting, PCR, and DNA sequencing; biochemical assays, such as detecting the presence of a protein product, for example, by immunological means (ELISAs and western blots) or by enzymatic function; plant part assays, such as leaf or root assays; and, by analyzing the phenotype of the whole plant.

Introgression of a trait into a plant genotype is achieved as the result of the process of backcross conversion. A plant genotype into which a trait has been introgressed may be referred to as a backcross converted genotype, line, inbred, or hybrid. Similarly, a plant genotype lacking the desired trait may be referred to as an unconverted genotype, line, inbred, or hybrid.

As used herein, the term “comprising” means “including but not limited to”.

Having described several embodiments in detail, it will be apparent that modifications, variations, and equivalent embodiments are possible. Furthermore, it should be appreciated that the examples in the present disclosure are provided as non-limiting examples.

### EXAMPLES

### Example 1: Evolving Dual-Trait EPSP Synthase Variants Using a Synthetic Yeast Selection System

The identification of a native, mutant EPSPS can take advantage of new gene editing technologies like CRISPR/Cas, TILLING, and others without employing traditional biotechnology transgenes. To date, only a small set of mutations are currently known to confer glyphosate resistance to native plant EPSPS and related homologs. However, these resistance-conferring mutations also reduce the net activity of this essential enzyme and thus lead to reduced plant growth under native expression. In enzymes of plant origin and similar homologs (termed Class I EPSPS enzymes), it is well-established that glyphosate insensitivity comes at a high detriment to the catalytic efficiency. Commonly evaluated mutations such as G101A, T102I, and P106S all lead to varying levels of improved glyphosate tolerance, but are not commercially viable as single mutants due to either hindered enzymatic efficiency for G101A and T102I or insufficient glyphosate tolerance for P106S. The double mutant T102I and P106S (denoted as TIPS), was one of the earliest commercially viable glyphosate-resistant EPSPS mutants of plant origin to be discovered. TIPS is a Class I enzyme that is essentially insensitive to glyphosate while still maintaining a reasonable Kₘ value for PEP. Even still, the turnover number (k_{cat}) is significantly reduced in TIPS and manifests as a growth deficit in plants.

In a more recent report, a glyphosate intolerant version of the Zea mays EPSPS with kinetics similar to that of bacterial Agrobacterium sp. strain CP4 gene was evolved by starting from the G101A mutant and discovering at least 17 stacked mutations in varying locations on the protein through multiple evolutionary rounds. Such a high number of mutations can be challenging to constitute in plants using gene editing techniques. Mutations that improve the catalytic efficiency of TIPS with fewer residue changes can establish a commercially viable glyphosate resistant EPSPS variant.

To address the need for identifying novel EPSPS variants, a more rapid screening system than in planta or in vitro experiments is required. However, the subtle growth deficits caused by known mutants in plants are not distinguishable in E. coli or other microbial model systems. As an example, the mutant G101A displayed very similar growth to wild-type EPSPS in an E. coli model even though this variant has only ~1% of the catalytic efficiency of the wild-type enzyme and shows a growth deficiency in plants.

The present example discloses the use of an eukaryotic model, Saccharomyces cerevisiae, as a more applicable host system for evolution of EPSPS variants. Yeast have already been used to study certain herbicide interactions, including herbicides targeting acetyl-CoA carboxylase and other metabolizing enzymes. Moreover, the high similarities in aromatic amino acid biosynthesis between yeast and plants can help lead to new targets for glyphosate tolerance. To establish this system, a synthetic strain of yeast was created that is dependent upon the heterologous expression of EPSPS for growth. Next, a directed evolution strategy was utilized to select for both glyphosate resistance and improved catalytic properties (i.e., k_{cat}/Kₘ) simultaneously through iterative selection strategies. It was demonstrated that additional mutations in the background of the TIPS mutant can greatly improve catalytic efficiency (k_{cat}/Kₘ) while maintaining high glyphosate insensitivity. In doing so, this example demonstrates the importance of synthetic selection strategies on the trajectory of enzyme evolution for dual-trait optimization.

### Materials and Methods

### Strains and Media

Yeast plasmids were propagated in E. coli DH10B. Saccharomyces cerevisiae strain BY4741 was obtained from the European Saccharomyces cerevisiae Archive for Functional Analysis (EUROSCARF, Y00000). Yeast genomic DNA was extracted using the Wizard Genomic DNA Purification Kit (Promega). Yeast cells were routinely grown in YPD, YSC, and YMM media. YMM medium was used for selections and characterization of EPSPS mutants and contains 1×YNB (6.7 g/L of Yeast Nitrogen Base (YNB, Difco)), 1× glucose (20 g/L), 1× Histidine, and 1× Methionine. Solid media contained 2.0% agar. Yeast and bacterial strains were stored at −80° C. in 20% glycerol in 2 mL Cryogenic Storage Vials (Fisherbrand).

### Transformations

E. coli transformations were routinely performed via electroporation with a BioRad Genepulser Xcell at 2.5 kV in 2 mm Electroporation Cuvettes (Bioexpress). Typically, 100 ng of cloning product either from Gibson assembly or ligation were added to 50 μL of competent cells. Transformations were rapidly recovered in 500 μL of SOC media. Recovered cultures were then plated or streaked on LB agar plates containing 100 μg/mL ampicillin and incubated overnight at 37° C. The next day, individual colonies were picked into 4 mL of LB media containing 100 μg/mL ampicillin and grown overnight at 30° C. The following day, plasmids were then isolated (GeneJET Plasmid Miniprep Kit, Thermo Scientific) and confirmed via Sanger sequencing.

Yeast transformations were routinely performed using Frozen EZ Yeast Transformation II Kit (Zymo Research) according to the manufacturer's instructions. In general, 25 μL of chemically competent S. cerevisiae BY4741 strains were transformed with 100-500 ng of plasmid DNA. The cells were incubated at 30° C. for 1-2 hours and then plated on selective media, either YSC-Ura, YSC-Leu, and YSC-Ura-Leu for 2 days at 30° C. Individual colonies were then picked at random into 1-2 mL of appropriate selective media and grown 2 days at 30° C. with 225 RPM orbital shaking.

### Plasmid and Strain Construction

Oligonucleotides were synthesized by Integrated DNA Technologies. PCR was performed using Q5 Hot Start polymerases (NEB). PCRs and other DNA products were cleaned using a NucleoSpin Gel and PCR Clean-up kit (Macherey Nagel). Vectors were digested using various restriction enzymes as noted (NEB) and prepared for ligation using Antarctic phosphatase (NEB). The low copy CEN/ARS vector backbone from a modified Mumberg collection p415-TEF1-PRM9t and p416-PTEF1-CYC1t with strong TEF1 promoter were used for all initial experiments unless otherwise noted (Crook, et al., Nucleic Acids Res. 39: e92 (2011); Mumberg, et al., Gene 156:119-122 (1995)). ScARO1 was PCR amplified from S. cerevisiae BY4741 genomic DNA and cloned into plasmid p415-PTEF1-PRM9t via the Gibson method for overexpression in S. cerevisiae (Gibson, Nat. Methods 6:343-345 (2009)).

ZmEPSPS was codon optimized for S. cerevisiae, synthesized as a gBlock (IDT), and cloned initially into plasmid p416-PTEF1-CYC1t. These plasmids are referred to as p415-TEF-ARO1mut (S. cerevisiae endogenous gene) and p416-TEF-EPSPS (Z. mays homolog). The TEF1 promoter was selected for these vectors because it is reported to be a strong promoter that exhibits moderately more consistent activity than other very strong promoters across different carbon sources and concentrations of glucose (Partow, et al., Yeast 27:955-964 (2010)). Promoters for the promoter series were either synthesized via oligonucleotide synthesis (IDT) for short core promoters Core1p and Core4p or amplified from the S. cerevisiae genome for STE5p and CYC1p (Redden and Alper, Nat. Commun. 6:7810 (2015)). The resulting plasmids are referred to as p416-Core1p (or other promoter)-EPSPS (low copy, CEN/ARS) and p415-TEF-ARO1mut (low copy, CEN/ARS).

### Mutagenesis Methods and Library Construction for Mutants of ARO1 and EPSPS

ScARO1 point mutants were generated using Agilent QuikChange Multi Site-Directed Mutagenesis Kit. ZmEPSPS was amplified using Agilent Mutazyme II to introduce mutations into the coding sequence (CDS) to generate mutagenesis pools. Two mutation frequencies were targeted; approximately 1-3 and 3-5 mutations per CDS length, denoted as low and medium frequency. The resulting PCR product pool was cloned into p416-Core1p through either Gibson assembly or ligation cloning with T4 DNA Ligase and transformed into Escherichia coli DH10B, where approximately 8×10⁵ to 4×10⁶ variants per library were harvested depending on the mutagenesis pool for transformation into yeast (Mumberg, et al., supra; Gibson, supra). DNA was isolated and sequenced from ten individual colonies and an average of 1-3 mutations per CDS for low frequency and 2-5 mutations per CDS for medium frequency was achieved for each library, respectively; limited wild-type template sequence was observed. The low copy plasmid p416-Core1p was selected as the recipient vector for this experiment to minimize the potential impact of plasmid copy number variation and to ensure maximize growth differentiation when comparing mutant EPSPS sequences (Karim, et al., FEMS Yeast Research 13:107-116 (2013)). After DNA isolation from E. coli, the library was transformed into S. cerevisiae strain sKR-024 using a high-efficiency lithium acetate protocol (Gietz and Schiestl, Nat. Protoc. 2:31-34 (2007)), yielding libraries of approximately 3×10⁵ to 2×10⁶ variants, depending on cloning and transformation efficiency.

### Flow Cytometry

Yeast cultures were either started directly from a random colony or from freezer stock and picked in triplicate into appropriate selective media, either YSC-Ura, YSC-Leu, and YSC-Ura-Leu. Cultures were grown for 48 hours at 30° C. to saturation phase. Fully grown yeast cultures were then diluted back to OD 0.01 and allowed to grow for 48 hours at 30° C. in a 96 deep-well plate shaker. Fluorescence was then analyzed via flow cytometry (BD Accuri C6 Flow Cytometer, BD Biosciences) at an excitation of 588 nm and emission of 633 nm for mKate2 RFP. Yeast populations of interest were then gated according to relative size and complexity using a logarithmic plot of side scatter (SSC) and forward scatter (FSC) and spectra were generated outlining RFP fluorescence intensity vs. cell count. FlowJo software was used to analyze all flow cytometry data.

### Directed Evolution Workflow

Yeast library pools were started from freezer stock and moved into yeast minimal media with or without glyphosate depending on the selection criteria applied. In general, library pools were diluted back to an OD of 0.01 and allowed to grow until saturation. Depending on mutants present in each pool this typically ranged from 48-96 hours. Once saturation phase was reached, a portion of the pools were freezer stocked in 20% glycerol and then sub-cultured into the next set of media, either containing or lacking glyphosate. Isolation of mutants was then performed by selecting random colonies on agar plates, either containing or lacking glyphosate as indicated in workflow diagrams. Unless otherwise indicated, final selections were all isolated on agar plates lacking glyphosate. EPSPS library ORFS were isolated from random colonies that survived on glyphosate selection on plates using a modified colony PCR method with a boiling step containing NaOH and Triton X-100. PCRs were confirmed via gel electrophoresis, cleaned then subjected to Sanger sequencing. Sequenced PCR products were subsequently re-amplified and transformed into a clean strain of S. cerevisiae sKR-024 for confirmation of phenotype in biological triplicate.

### In Vivo Characterization of EPSPS Variants and Growth Rate Analysis

EPSPS variants were re-cloned into a clean p416-Core1p backbone and transformed into strain sKR-024 as described above. Colonies were then picked in triplicate into YSC-Ura-Leu and outgrown for 2 days at 30° C. Cultures were then diluted back to OD 0.001 in yeast minimal media (YMM) containing varying amounts of glyphosate as indicated. Growth rate measurements were then obtained using an Infinite M200 PRO microplate reader (Tecan, Männedorf, Switzerland) with incubation at 30° C. for 72-120 hours depending on growth rate of selected variants.

### Statistical Analyses

All statistical analyses were performed using GraphPad Prism's built-in functions. One way analysis of variance (ANOVA) was used to determine statistical differences between group means. Pairs of group means were compared Tukey's Post-Hoc test. Number of biological replicates and significance levels are highlighted as appropriate.

### In Vitro Characterization of EPSPS Mutants

Activity was determined by quantifying release of Pᵢ, coupled to a reaction with MESG, catalyzed by purine-nucleoside phosphorylase using the EnzChek Phosphate Assay Kit from Invitrogen, first published in 1992. 95 μL of master mix was added to each well and 5 μL of the varied substrate dilutions were multi-channeled into the plate to start the reaction simultaneously for all conditions. Absorbance measurements at 360 nm were taken every 10 seconds for 3 minutes to capture the initial reaction rate of each condition. Varying levels of substrate were added to generate the resulting reaction rate curves. Each condition was performed in triplicate. Kᵢ was determined as previously described (Lu, et al., Protein Engineering, Design and Selection 30:395-399 (2017)). Data was fit to the Michaelis-Menten kinetics equation and were also validated through GraphPad Prism's built-in function for each variant.

### Protein Expression and Purification

For protein expression of EPSPS and its variants, E. coli BL21 (DE3) was used as the expression host. Electrocompetent cells were transformed with the corresponding constructed plasmids, pET28a (+) with selected EPSPS variants (wild-type, TIPS, TIPS P126S, TIPS K296R, TIPS P126S/K296R). A single colony of an E. coli BL21 (DE3) strain harboring one of the constructed plasmids was inoculated into 2 mL of Luria Bertani broth (LB) medium with 50 μg/mL kanamycin and grown overnight at 37° C./225 rpm. The overnight-grown culture (using 100 μl) was scaled up with 2000-fold dilution in a 500-mL triple baffled shake flask and grown to a cell density of 0.8 (optical density [OD600]) at 37° C./225 rpm. Protein expression was induced by adding 0.2 mM of isopropyl β-D-1-thiogalactopyranoside (IPTG) and cells were cultured for 24 hours at 20° C./225 rpm. The induced cell culture was harvested by centrifugation at 4,000 g and 4° C. for 20 mins. Cell pellets were then resuspended in 25 mL of Dulbecco's Phosphate Buffered Saline (DPBS) (Thermo Fisher Scientific, Waltham, MA) pH 7.0 buffer containing 10 mM imidazole, 1 g/L of lysozyme and 5 μl of Pierce™ Universal Nuclease (Thermo Fisher Scientific, Waltham, MA), followed by mixing on a rocker for 30 mins at 4° C. Subsequently, cells were lysed by sonication and the resulting cell lysate was centrifuged at 14,000 g and 4° C. for 20 minutes to obtain the supernatant that contains soluble proteins. Target proteins from the supernatant was purified by HisPur™ Ni-NTA Resin (Thermo Fisher Scientific, Waltham, MA) according to the manufacturer's instruction.

The eluate was dialyzed with 3C protease added to the dialysis cassette, into the appropriate buffer followed by size-exclusion fast protein liquid chromatography. All EPSP synthase variants were stored in 20 mM Tris (pH 7.5), 100 mM NaCl and 10 mM β-mercaptoethanol. The protein concentration was then determined by using the Coomassie Plus Bradford Assay kit (Thermo Fisher Scientific) and the Infinite M200 PRO microplate reader (Tecan, Männedorf, Switzerland) to measure the absorbance of assay mixtures. The presence and purity of the purified proteins were assessed by sodium dodecyl sulfate-polyacrylamide gel electrophoresis.

### Protein Crystallization

To identify crystallization conditions of Zea mays EPSP synthase variants, 20 mg/ml purified wild-type EPSP synthase sample was pre-incubated with 5 mM shikimate-3-phosphate and 5 mM glyphosate for 3 hours at 4° C., and then screened with sparse-matrix screening using Phoenix robotic system (Art Robinson). The initial crystallization hit was identified with rod-shaped crystals appearing after incubating screening plates at 25° C. for three days. The crystallization conditions for all EPSP synthase variants were optimized as 0.1M MES, 25% PEG 8000, pH 6.5, set up as sitting-drop vapor diffusion. All sitting drops were set up by mixing 1.33 μl of reservoir solution and 0.67 μl of 20 mg/ml protein sample.

### X-Ray Diffraction Data Collection, Data Processing, and Model Refinement

Individual EPSP synthase crystals were flash-frozen in liquid nitrogen after being cryoprotected with 30% (v/v) glycerol. X-ray diffraction data were collected at the 23-ID-B beamline in Advanced Photon Source (Lemont, IL). The X-ray diffraction patterns for all EPSP synthase crystals were processed to ~2 Å resolution using HKL2000 (Otwinowski and Minor, Meth. Enzymol. 276:307-326 (1997)). The wild-type EPSP synthase structure was solved by molecular replacement with EPSP synthase from E. coli as the initial search model (PDB code 1G6T). The molecular replacement solution for wild-type EPSP synthase was iteratively built and refined using Coot (Emsley, et al., Acta Crystallogr. D Biol. Crystallogr. 66:486-501 (2010)) and Phenix (Liebschner, Acta Cryst. D 75:861-877 (2019)) refinement packages. Crystal structures of other variants were solved by molecular replacement with wild-type EPSP synthase structure and iteratively refined. Procheck and MolProbity evaluated the quality of the finalized EPSP synthase structures. The final diffraction data collection and structural determination statistics arc shown in Table 2.

TABLE 2

| | | | | |
|---|---|---|---|---|
| | | | | TIPS P126S |
| WT | TIPS | TIPS P126S | TIPS K296R | K296R |

Data collection

| | | | | | |
|---|---|---|---|---|---|
| Space group | P1 | P1 | P1 | P1 | P1 |

Cell dimensions

| | | | | | |
|---|---|---|---|---|---|
| a, b, c (Å) | 46.52, 63.28, 69.96 | 46.57, 57.95, 72.91 | 46.53, 63.55, 70.23 | 46.17, 62.71, 68.43 | 46.32, 63.11, 69.79 |
| α, β, γ (°) | 84.76, 85.51, 85.55 | 86.52, 81.56, 79.62 | 85.87, 85.78, 85.50 | 86.54, 86.35, 86.68 | 85.36, 85.84, 85.53 |
| Resolution (Å) | 50.00-2.25 | 50.00-1.90 | 50.00-2.27 | 50.00-2.28 | 50.00-1.90 |
| | (2.29-2.25)* | (1.93-1.90) | (2.31-2.27) | (2.32-2.28) | (1.93-1.90) |
| R_{sym} | 0.052 (0.163) | 0.082 (0.249) | 0.061 (0.141) | 0.098 (0.254) | 0.066 (0.162) |
| Rₚᵢₘ | 0.049 (0.151) | 0.076 (0.240) | 0.042 (0.095) | 0.059 (0.172) | 0.044 (0.111) |
| CC ½ ^{Y} | 0.978 (0.933) | 0.951 (0.880) | 0.986 (0.966) | 0.982 (0.920) | 0.981 (0.954) |
| I/σ | 11.2 (3.5) | 6.9 (1.6) | 17.1 (6.3) | 8.9 (2.3) | 16.3 (4.3) |
| Completeness (%) | 90.1 (79.9) | 84.9 (83.1) | 89.0 (87.7) | 90.5 (88.3) | 90.2 (91.5) |
| Redundancy | 2.0 (1.9) | 1.8 (1.7) | 3.0 (3.0) | 3.6 (2.9) | 3.1 (2.9) |

Refinement

| | | | | | |
|---|---|---|---|---|---|
| Resolution (Å) | 48.74-1.93 | 45.27-1.90 | 48.91-1.83 | 44.60-2.18 | 39.62-1.60 |
| | (2.00-1.93) | (1.97-1.90) | (1.89-1.83) | (2.25-2.18) | (1.66-1.60) |
| No. reflections | 50845 (4129) | 49633 (4760) | 61556 (5474) | 34616 (2602) | 86456 (5925) |
| R_{work} | 0.1676 (0.2381) | 0.1562 (0.2029) | 0.1522 (0.2034) | 0.1490 (0.1826) | 0.1523 (0.2045) |
| R_{free}^{±} | 0.2037 (0.3093) | 0.1971 (0.2433) | 0.1823 (0.2575) | 0.1972 (0.2528) | 0.1759 (0.2388) |
| No. atoms | 7315 | 7235 | 7358 | 6997 | 7429 |
| Protein | 6592 | 6483 | 6570 | 6555 | 6613 |
| Ligand/ion | 64 | 52 | 64 | 52 | 82 |
| Water | 659 | 700 | 724 | 390 | 734 |

B-factors (Å²)

| | | | | | |
|---|---|---|---|---|---|
| Protein | 20.3 | 15.5 | 17.5 | 22.2 | 17.2 |
| Ligand/ion | 17.3 | 12.8 | 19 | 21.9 | 19.6 |
| Water | 30.2 | 27.8 | 30.1 | 29.7 | 30.9 |

R.m.s. deviations

| | | | | | |
|---|---|---|---|---|---|
| Bond lengths (Å) | 0.0043 | 0.0065 | 0.0049 | 0.0069 | 0.0056 |
| Bond angles (°) | 0.73 | 0.82 | 0.79 | 0.89 | 0.85 |

Ramachandran plot

| | | | | | |
|---|---|---|---|---|---|
| Favored | 97.39% | 98.04% | 97.39% | 98.17% | 97.73% |
| Allowed | 2.61% | 1.96% | 2.61% | 1.83% | 2.27% |
| Outliers | 0.00% | 0.00% | 0.00% | 0.00% | 0.00% |
| Molprobity | 1.30 / 99th | 1.12 / 100th | 1.24 / 99th | 1.43 / 99th | 1.08 / 99th |
| score\hat{ } | percentile | percentile | percentile | percentile | percentile |

*Values for the corresponding parameters in the outermost shell in parenthesis.
^{Y} CC_{1/2} is the Pearson correlation coefficient for a random half of the data, the two numbers represent the lowest and highest resolution shell, respectively.
^{±}R_{free} is the R_{work} calculated for about 10% of the reflections randomly selected and omitted from refinement.
\hat{ }MolProbity score is calculated by combining clashscore with rotamer and Ramachandran percentage and scaled based on X-ray resolution. The percentage is calculated with 100th percentile as the best and 0th percentile as the worst among structures of comparable resolution.

### Results

Creating an S. cerevisiae Selection Host Strain Dependent on EPSPS Expression

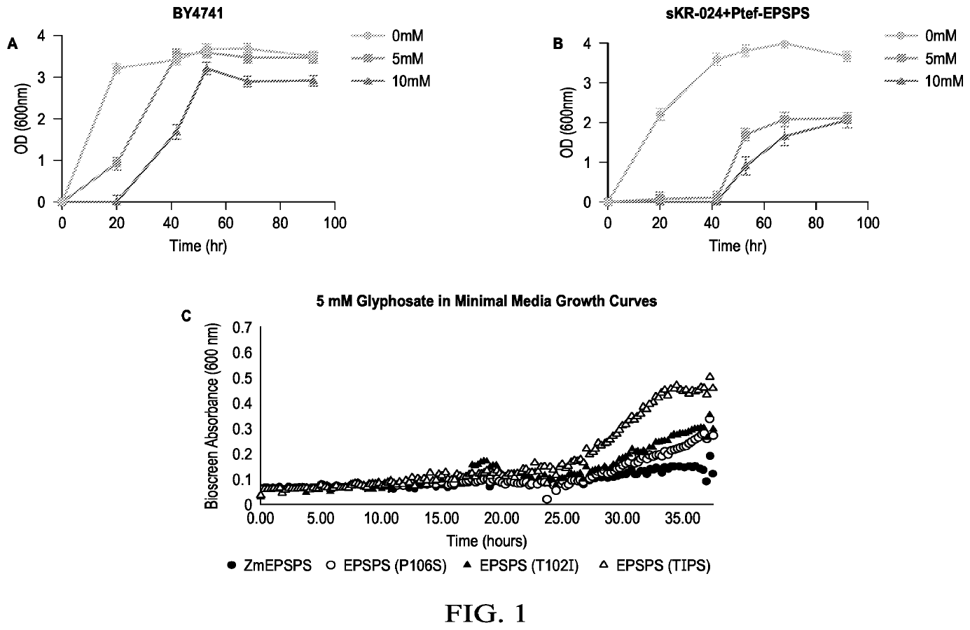

Previous research has primarily focused on E. coli as a chassis for selecting improved EPSP synthases. The present example details the development of S. cerevisiae as a model eukaryotic system for glyphosate resistance, thus requiring synthetically addicting this cell to the expression and activity of EPSPS in an effort to create a growth-based selection scheme. Yeast have previously been reported to be resistant to glyphosate. Validating this finding, it was found that wild-type S. cerevisiae BY4741 is relatively insensitive to high quantities of glyphosate and showed slight growth inhibition at 5 mM and 10 mM concentrations, thus suggesting that the native EPSP synthase subunit of ScARO1 is not inhibited strongly by the compound (FIG. 1).

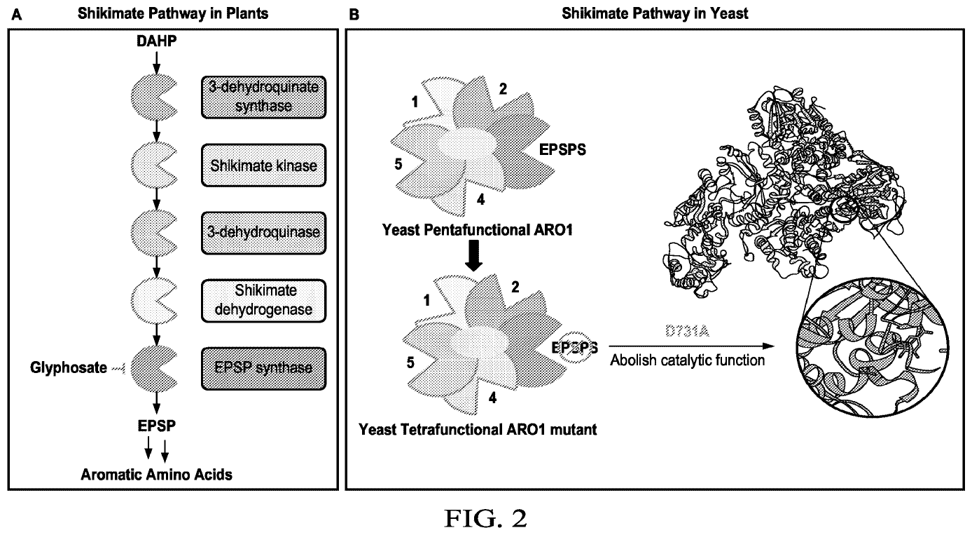

In order to create a selection scheme, it was necessary to remove the native function of EPSPS and complement a mutant version. However, since yeast ARO1 is pentafunctional and the EPSPS function is in the middle of this protein, the elimination of function using previously developed approaches such as truncations were not feasible (FIG. 2). Eliminating ARO1 EPSPS function was accomplished through testing a series of alanine mutations and screening on media lacking aromatic amino acids. To guide mutation selection, a homology model for yeast ARO1p was generated using the structurally similar pentafunctional AROM complex from Chaetomium thermophilum as a template (FIG. 2). Using this model, structural data from similar EPSPS enzymes were assessed to identify mutations that were likely to disrupt only the EPSPS function while keeping the other four enzyme functions intact. Through this analysis, mutations at residues D731 and C853 as well as a triple mutant composed of residues K425, S426, and R430 were evaluated. This analysis suggested that the single mutants each disrupt a purported vital residue in the reaction mechanism and the triple mutant acted to disrupt the entire active site.

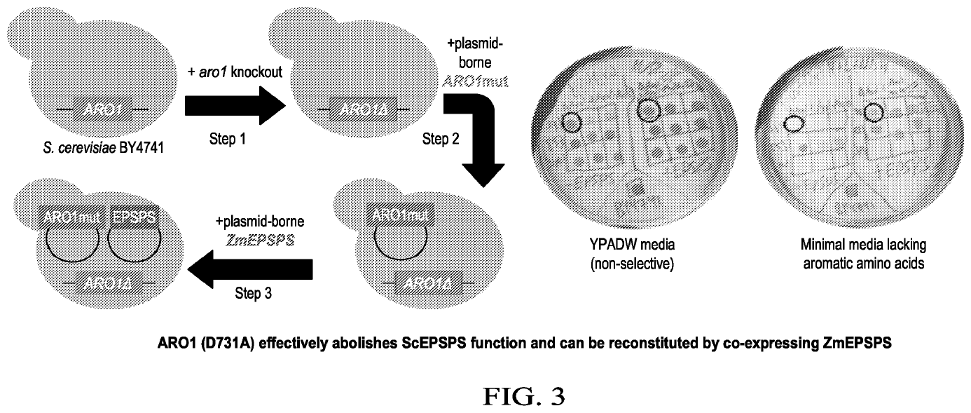

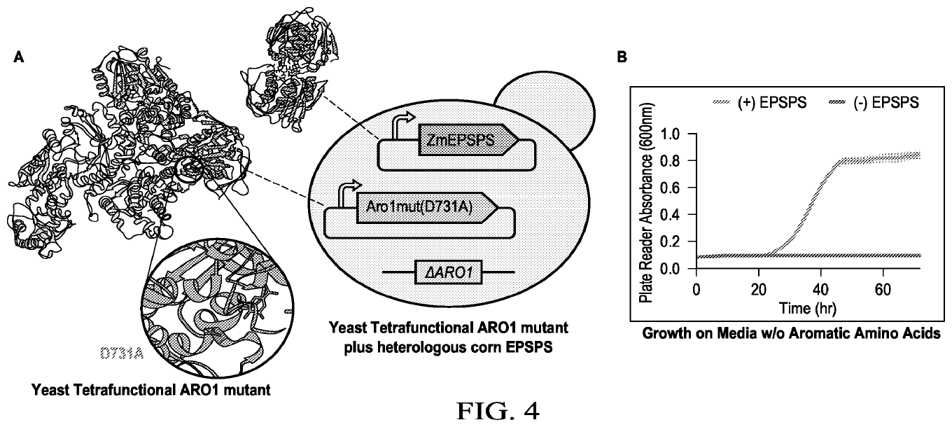

Each of the three identified aro1 mutants was expressed on a plasmid (p415-PTEF-PRM9t) in a deletion strain, S. cerevisiae Δaro1::KanMX. The resulting strains were then plated on solid media with and without aromatic amino acids to test for growth complementation. Both D731A and the triple alanine substitution mutant did not grow on media lacking aromatic amino acids (FIG. 3), whereas C853A retained its native activity and thus was not a viable option. Having identified a putative EPSPS-null allele, the same strains were transformed with a plasmid containing wild-type EPSPS from Zea mays (ZmEPSPS) to see if the aromatic amino acid synthesis capacity can be revived through complementation. Expression of aro1-D731A effectively abolishes only ScEPSPS function and the growth can be reconstituted by complementing ZmEPSPS. This final strain, containing the knockout of native aro1 function and replacement with p415-PTEF-aro1 (D731A)-PRM9t, was grown in liquid media with and without heterologous ZmEPSPS expressed to validate the desired growth-based selection strategy (FIG. 4) and was thus used for all future experiments in this example. This EPSPS dependent yeast (denoted as sKR-024) serves as a synthetic yeast model chassis to study known mutants and discover new high functional variants.

### Characterizing the Synthetic EPSPS Mutation Screening System

After developing the ZmEPSPS-dependent strain, next it was sought to confirm whether this complementation could elicit and recapitulate glyphosate sensitivity and plant responses in S. cerevisiae. To do so, strain sKR-024 was grown in varying glyphosate concentrations to determine sensitivity. This strain indeed displayed a growth deficit in glyphosate compared to WT BY4741, but over time was able to eventually show growth even at high glyphosate concentrations (FIG. 1). This escape phenotype is likely due to the high expression of ZmEPSPS achieved here with the strong TEF1 promoter and parallels previous literature that observed improved glyphosate resistance in some weeds that have developed high expression of wild-type EPSPS. This observation suggests that this synthetic yeast system can recapitulate accurate growth trends from plants.

To determine if this system could further corroborate observations from previous literature, the well-studied EPSPS mutants T102I and P106S, and the combined double mutant TIPS (T102I-P106S) were generated and complemented into this yeast system. These mutations were reported to result in varying degrees of glyphosate resistance. As displayed in FIG. 1 (Panel C), it was confirmed that experimental growth data in yeast generally matched the literature trends related to glyphosate tolerance for these EPSPS variants with P106S<T102I<TIPS.

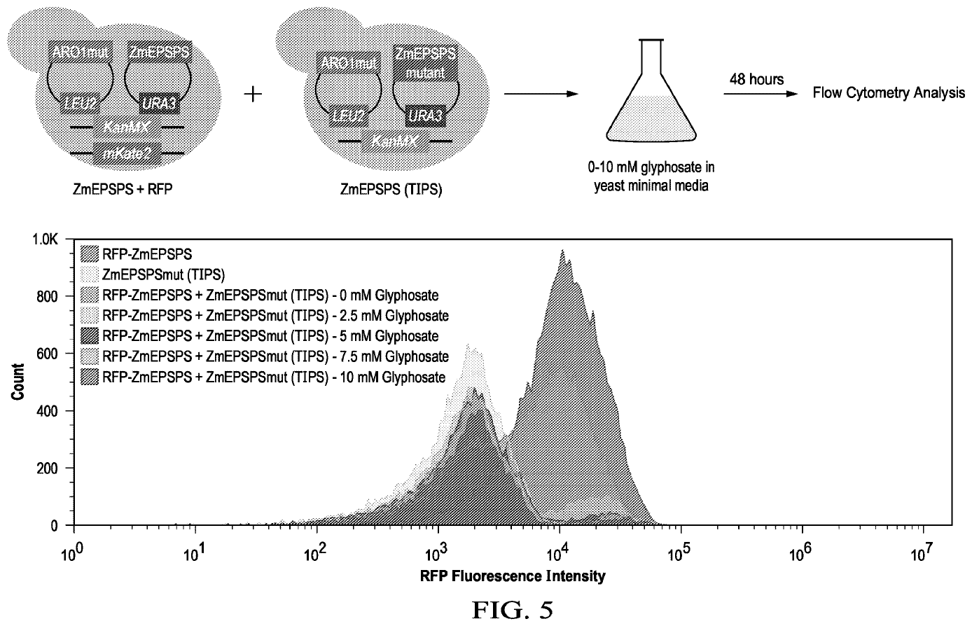

Next, it was sought to demonstrate that this difference in tolerance led to a selectable phenotype. To do so, a mock selection for glyphosate resistance was achieved by transforming wild-type EPSPS into sKR-024 containing an RFP cassette and TIPS into sKR-024 without an RFP cassette to enable flow cytometry tracking of population distributions. A 50:50 starting inoculum of each strain was cultured in yeast minimal media containing 0, 2.5, 5, 7.5, or 10 mM glyphosate. Yeast containing wild-type EPSPS (RFP fluorescence around 104 RFU) began to decrease in count rapidly after the addition of glyphosate in a titratable manner with glyphosate (FIG. 5). On the other hand, the non-fluorescent strain containing the TIPS variant showed consistent cell count across multiple levels of glyphosate as predicted by its significantly higher Kᵢ for glyphosate.

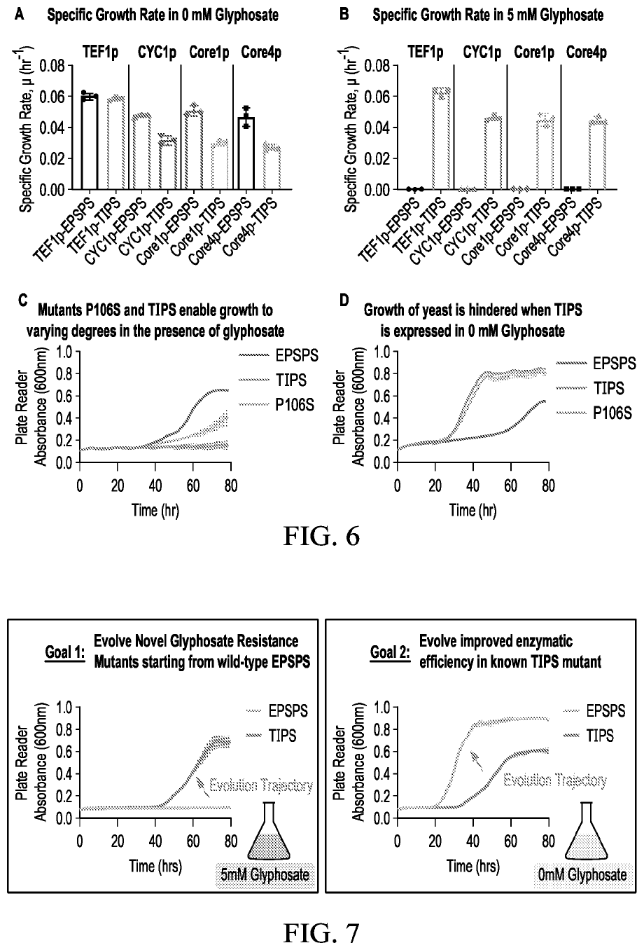

A final observation from this initial mock selection experiment was the consistently lower cell count of TIPS relative to wild-type EPSPS in the absence of glyphosate, thus suggestive of a lower overall fitness with TIPS. Previous literature has reported that mutants resulting in higher glyphosate tolerance negatively impacted Kₘ and/or k_{cat}. In plant models, this phenotypically leads to a significant growth deficit in EPSPS mutant expressing plants when glyphosate is not present. To further explore this phenotype (and enable a more powerful dual-selection strategy), it was examined if lower expression of the complemented EPSPS enzyme could further exacerbate this deficit and lead to stronger selection pressures. To do so, a synthetic promoter series was constructed consisting of (in order of reported expression level) TEF1p, CYC1p, STE5p, and synthetic minimal yeast promoters Core1p and Core4p. In doing so, it was found that weaker expression of EPSPS and TIPS did correlate to a lower specific growth rate, longer lag times, and often lower final OD values (FIG. 6). However, the reduction in growth rate was much greater for TIPS than for EPSPS when the expression strength was weakened (FIG. 6, Panel A). Thus, this weaker expression exploits the kinetic deficiency of TIPS and provides a window of selection for discovering EPSPS variants stemming from a TIPS background (and other backgrounds). As synthetic promoter Core1p generated the greatest difference in growth rates between EPSPS and TIPS, it was chosen for all future selection studies.

Having established low copy number and weak promoter expression of EPSPS variants, it was sought to recharacterize phenotypes under field conditions that are used in planta. FIG. 6, Panels C and D, thus shows the observed growth characteristics of the present synthetic yeast model with expression of wild-type EPSPS, P106S, and T102I-P106S (TIPS) in cither 0.5 mM or 0 mM glyphosate. It has been reported previously that under standard glyphosate foliar application, 0.3 mM or more of glyphosate (gly) can accumulate in a plant's root nodules and meristems. In yeast minimal media (0 mM gly), both EPSPS and P106S grew very similarly, supporting the fact that they have similar kinetic properties. However, TIPS grew at a significant deficit, as seen in plant hosts. In 0.5 mM gly on the other hand, EPSPS was completely inhibited, showing no growth, while P106S showed slightly higher growth than EPSPS after 80 hours, supporting its 5-fold higher Kᵢ value for glyphosate. TIPS, which was highly insensitive to glyphosate, grew well in the presence of glyphosate, yet still showed less than WT overall growth owing to low catalytic efficiency. These trends support reported literature of mutants P106S and TIPS grown in various plant hosts. As a result, the present synthetic yeast model system closely resembles in planta observations and thus can be used to select for dual-trait EPSPS variants aimed at both enzymatic efficiency and glyphosate resistance.

### Flexible Selections for the Directed Evolution of EPSP Synthases Using a Synthetic Yeast System

With a selection host in place, it was next sought to evaluate various directed evolution strategies to generate novel variants of corn EPSPS. Mutagenesis libraries were generated using both EPSPS and TIPS variant as background scaffolds. These mutagenic pools were placed under selection separately to observe their independent outcomes towards the goal(s) of improved glyphosate-resistance and/or improved overall catalytic activity (FIG. 7). Different selection regimes were evaluated for their mutational and phenotypic outcomes, leading to confirmation that through flexible selection criteria (i.e., varying the number of subcultures in non-glyphosate media vs. glyphosate media), it is possible to bias the evolutionary outcomes.

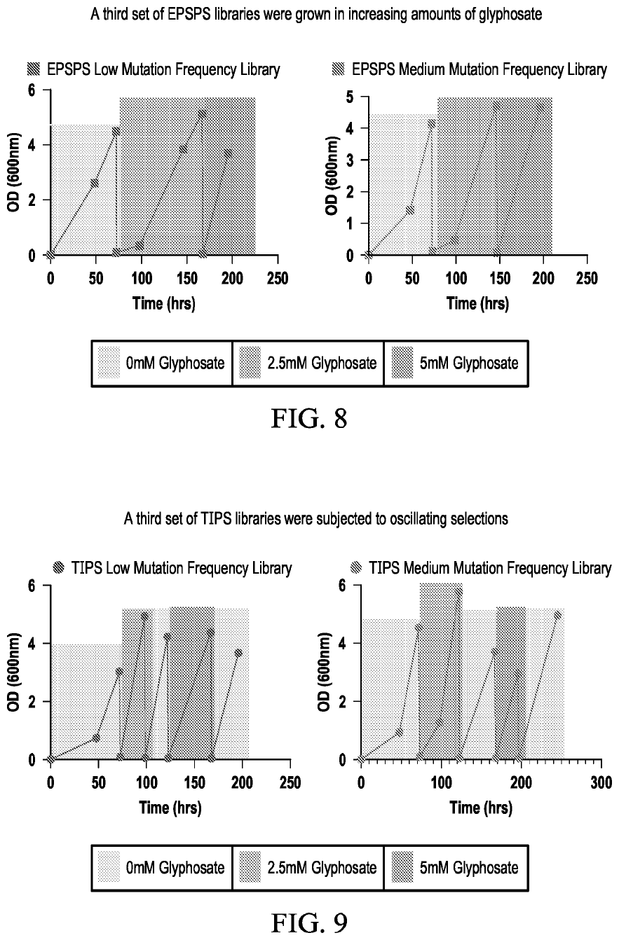

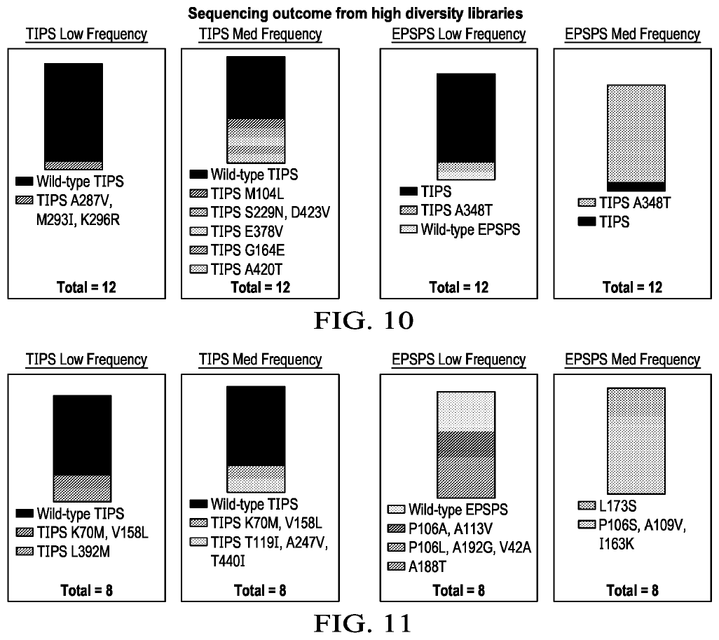

As a first pass, mutagenesis libraries from both EPSPS and TIPS background were grown in increasing amounts of glyphosate, starting with 0 mM and ending with 5 mM (FIG. 8 and FIG. 9). For EPSPS, library pools were simply grown in the presence of increasing levels of glyphosate thinking that wild-type EPSPS already confers high growth in non-glyphosate and thus improved tolerance was necessary. In the case of TIPS, oscillating between glyphosate and non-glyphosate conditions could prioritize mutations that confer improved growth in non-glyphosate media while maintaining a relatively high glyphosate tolerance already seen in this variant. Multiple, parallel pools from these backgrounds were placed in separate flasks under these selection criteria to observe mutational outcomes (FIG. 10 and FIG. 11).

For the EPSPS library pools, a few new mutations were discovered including A188T, which is in an alpha helix close to P106S and located in a position that may play a significant role in the conformational change that occurs when both substrates, PEP and S3P, are bound. However, mutations with the well-studied proline to serine mutation (P106S) dominated the EPSPS background, thus showcasing the strength of the present synthetic yeast model for selection of glyphosate-resistant mutants. For the TIPS mutagenesis pools, a few new mutations appeared in this initial first pass, yet the selection outcomes were primarily dominated by TIPS. As a result, a better set of outgrowth criteria would balance the selection poles, since growth in non-glyphosate seemingly has a lower selective pressure between EPSPS and TIPS than glyphosate-containing conditions.

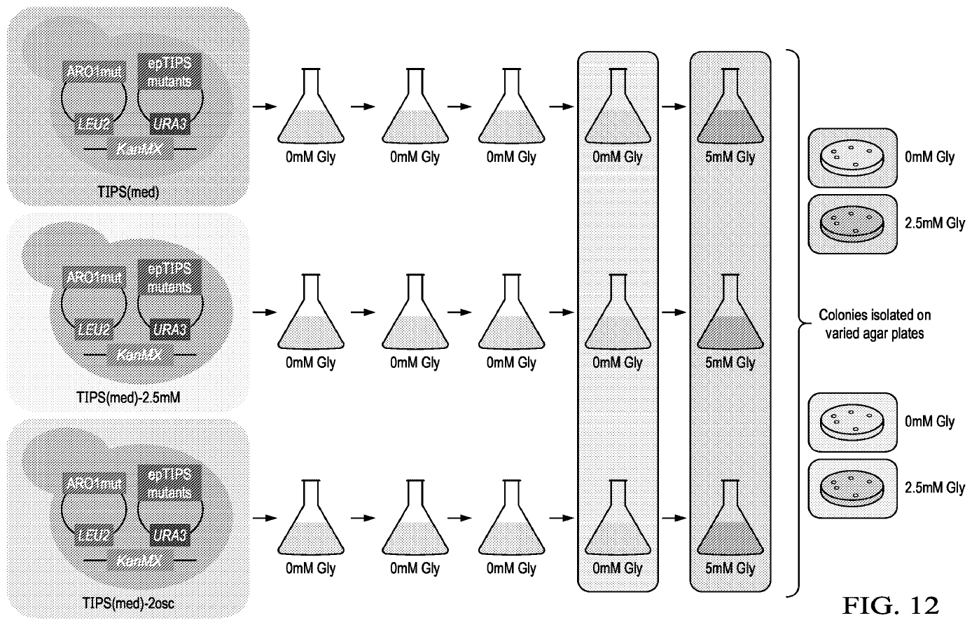

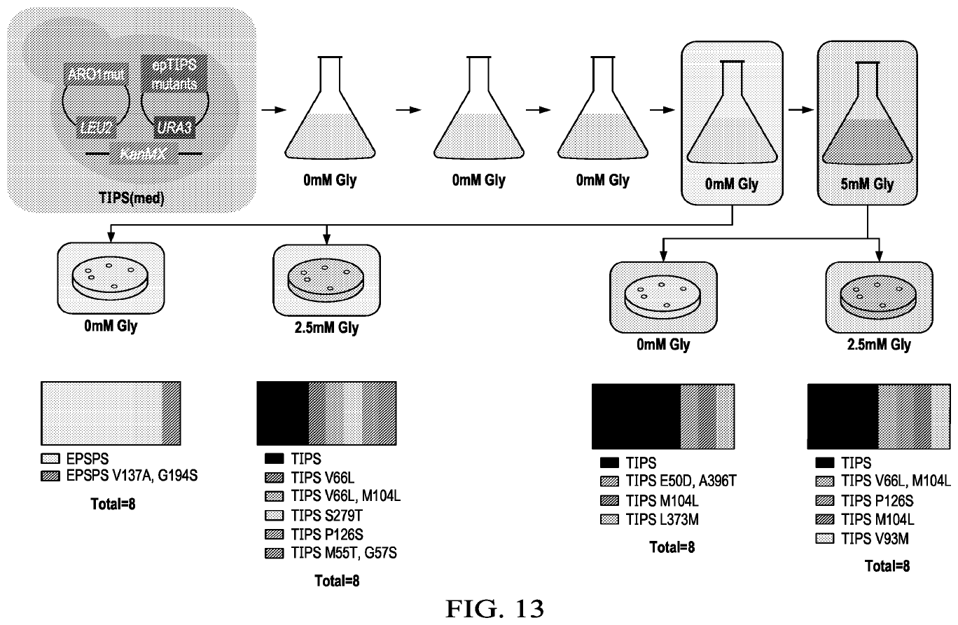

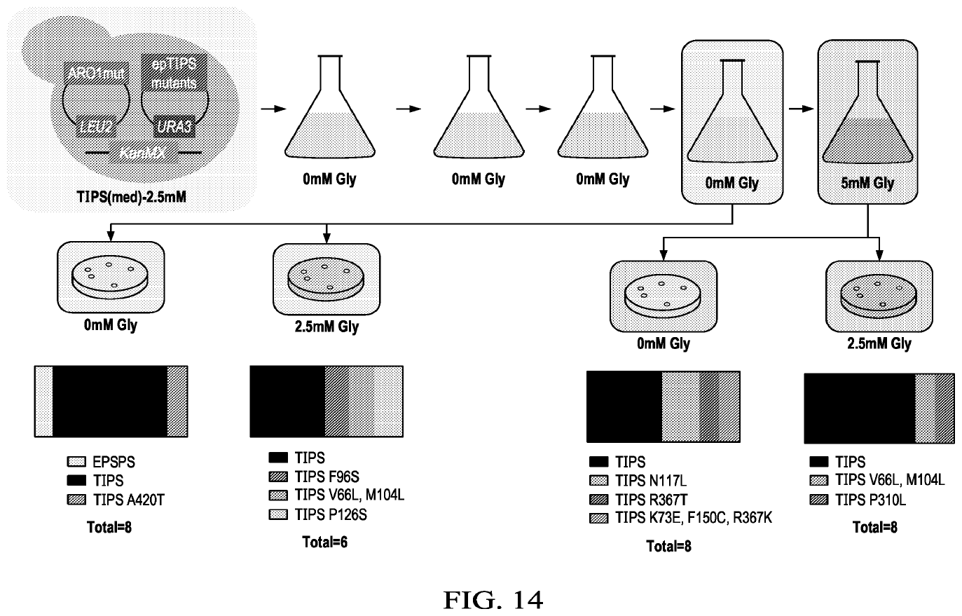

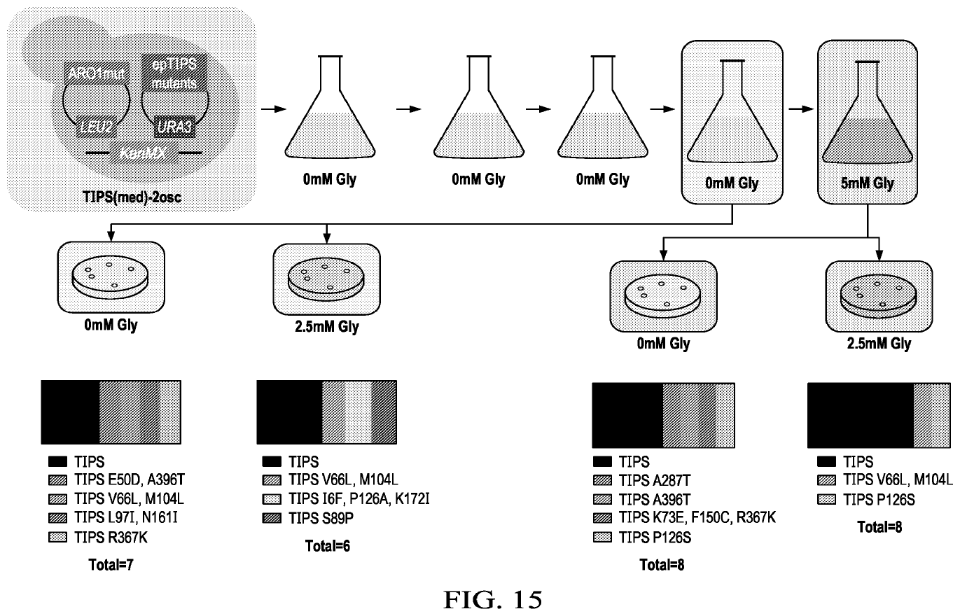

To achieve a better balance, multiple outgrowths in non-glyphosate containing media were studied, ending with 1-2 outgrowths. To do so, one of the cultures was a fresh library, TIPS (med), with no exposure to any strong selections, one culture was the same library that had a single outgrowth in 2.5 mM glyphosate, and the last was the outcome of the oscillating selection ending on 2.5 mM glyphosate from FIG. 9 (schematically depicted in FIG. 12). Through this experiment, it was sought to evaluate if using various starting points would lead to variations in the outcomes and inform a better directed evolution campaign for dual-trait evolution. Several interesting observations emerged from the outcomes of these selection strategies (FIG. 13, FIG. 14 and FIG. 15). First, it was possible to revert the TIPS mutant back to wild-type EPSPS by selecting in 0 mM for 4 subcultures followed by plating on 0 mM glyphosate agar plates. This shows the utility of this selection pole for improved basal enzymatic traits and activity. Second, it was necessary to have a single outgrowth on high levels of glyphosate in either liquid media or agar to completely eliminate any variants with low glyphosate tolerance, validating the strength of this selection pole in comparison to the non-glyphosate selection pole. More subtle titrations with weaker content (such as 0.25 mM) could further improve this selection. Third, it was observed that the resulting mutants varied depending on the exact selection pressure and evolutionary trajectory, suggesting that each approach generates slightly different traits in glyphosate and non-glyphosate media. With it established that balancing the selection poles is important to deliver tailored protein traits, a more informed directed evolution campaign strategy was undertaken.

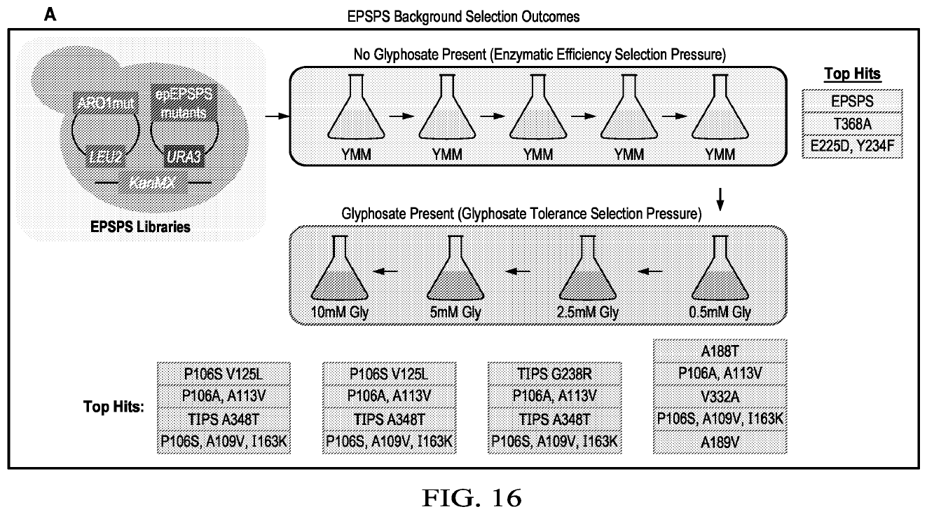

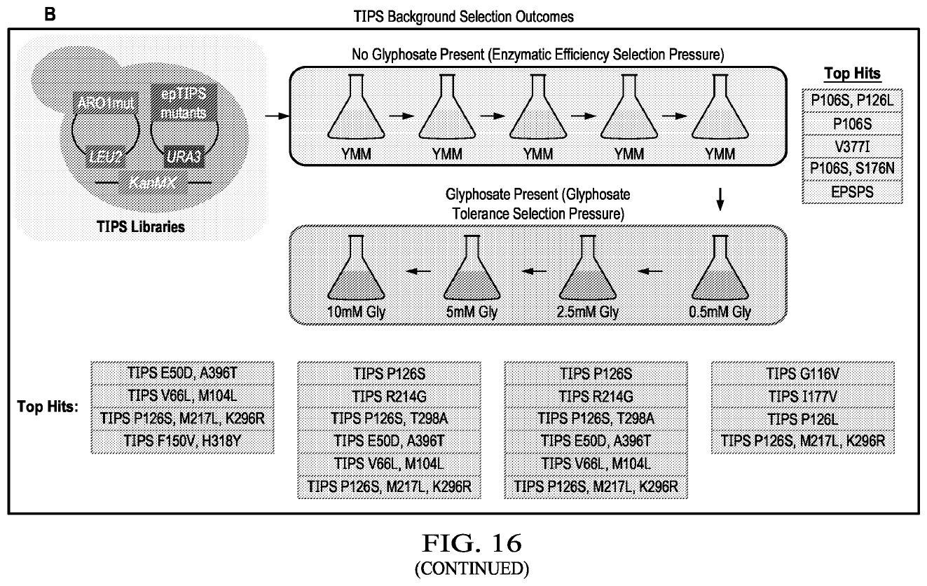

A final directed evolution campaign was then pursued with the same library mutagenesis pools starting from either EPSPS or TIPS genetic background using the optimized selection strategy shown in FIG. 16. The first stage selects for high functioning EPSPS variants that confer strong growth characteristics and the second stage subcultures into increasing levels of glyphosate to provide a graded selection pressure to unearth glyphosate tolerant mutants. The top hits from each stage of these selections are shown in FIG. 16. Depending on the selection pressure applied, different outcomes were observed. For example, from the EPSPS background, mutants P106S-V125L and P106A-A113V emerged from the high glyphosate (10 mM glyphosate) selections, whereas single mutations such as V332A and A188T emerged from lower glyphosate (0.5 mM glyphosate) selections. Interestingly, significant improvements in EPSPS under non-glyphosate conditions were not observed, although a few mutants such as T368A and E225D-Y234F were isolated after multiple outgrowths in non-glyphosate media. Presumably, selection in non-glyphosate could discover variants stemming from wild-type EPSPS with better enzymatic efficiency, although these mutations were not observed. For the TIPS background library pools, a large range of mutational outcomes were observed. Under non-glyphosate selection, TIPS was either reverted to variants containing only P106S or entirely back to wild-type EPSPS, along with a few other single mutations such as V377I.

When the pools were moved back into glyphosate containing media, all resulting variants contained the TIPS mutation plus a few other additional mutations. Of note were mutants TIPS-V66L-M104L and TIPS-P126S-M217L-K296R, which were highly dominant in their respective pools and were not observed in the first pass unbalanced oscillating selection.

By employing such a bespoke dual-trait selection strategy that balanced the two selection poles, novel mutations were discovered that were isolated in greater abundance than the starting points, EPSPS and TIPS. Interestingly, single mutations that have been previously reported to have high glyphosate insensitivity but extremely low catalytic efficiency, such as G101A and T102I, were not observed from any of the selection pools, further validating the strength of the present selection strategy to enable dual-traits phenotypes.

In Vivo Characterization of Novel Mutants from Varied Selection Criteria

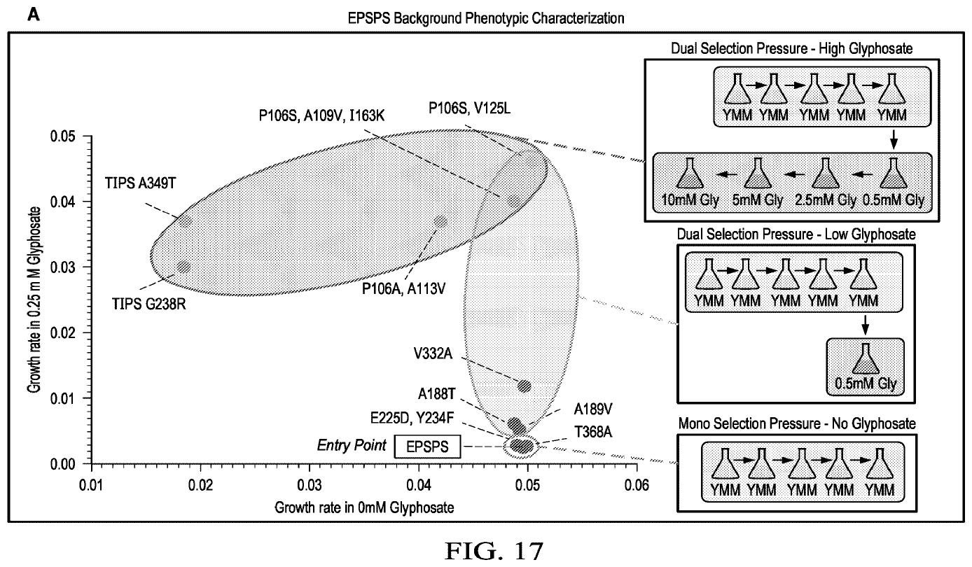

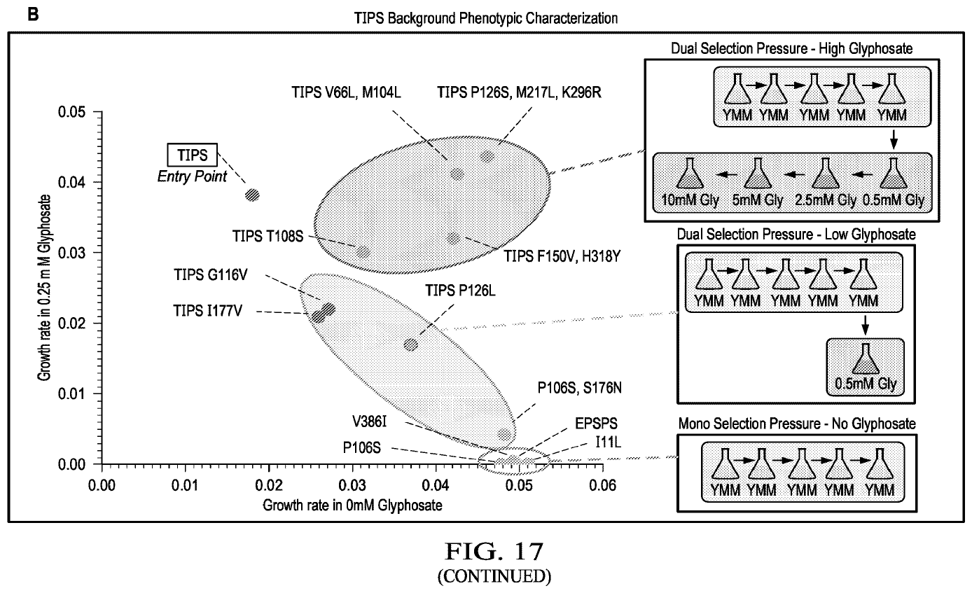

Select mutants isolated from the final directed evolution campaign with the improved selection strategy were further evaluated in assorted glyphosate concentrations to determine growth characteristics. Mutants isolated from an EPSPS background were evaluated in media containing 0 mM and 0.25 mM glyphosate with the latter being the concentration at which wild-type EPSPS cannot grow after 100 hours. Mutants isolated from a TIPS background were evaluated in media containing 0 mM and 5 mM glyphosate, since the goal for this campaign was to maintain high glyphosate tolerance while enhancing catalytic efficiency. These results are shown in FIG. 17 as a phenotypic landscape. The non-glyphosate mono selection highlighted in blue represents mutations resulting from 5 subcultures in 0 mM glyphosate. The low glyphosate dual selection highlighted in light green represents mutants resulting from an additional subculture into 0.5 mM glyphosate. Lastly, the high glyphosate dual selection highlighted in dark green represent mutations isolated after subcultures in increasing levels of glyphosate, up to 10 mM.

These results demonstrate that flexible selection criteria can generate tailored enzymes. For example, pools grown solely in yeast minimal media without glyphosate give phenotypes that tend to concentrate in the lower right quadrant of the phenotypic landscape where normal enzyme activity and growth characteristics dominate. For EPSPS, this resulted in the isolation of wild-type EPSPS along with a few other mutations with similar growth characteristics to EPSPS. For TIPS, reversions of the T102I or even complete reversion back to EPSPS was seen in selections that overly biased improved catalytic efficiency. Interestingly, the single subculture in 0.5 mM glyphosate, representing a low glyphosate dual selection, resulted in new mutants that did not dominate in the 0 mM glyphosate mono selection but have moderate glyphosate tolerance, such as A118T and V332A from the EPSPS background and TIPS-P126L and TIPS-1177V from the TIPS background. Lastly, selection in higher concentrations of glyphosate (2.5, 5, and 10 mM) resulted primarily in mutants that retain high glyphosate tolerance and also high growth in non-glyphosate, such as TIPS-V66L-M104L and TIPS-P126S-M217L-K296R from the TIPS background.

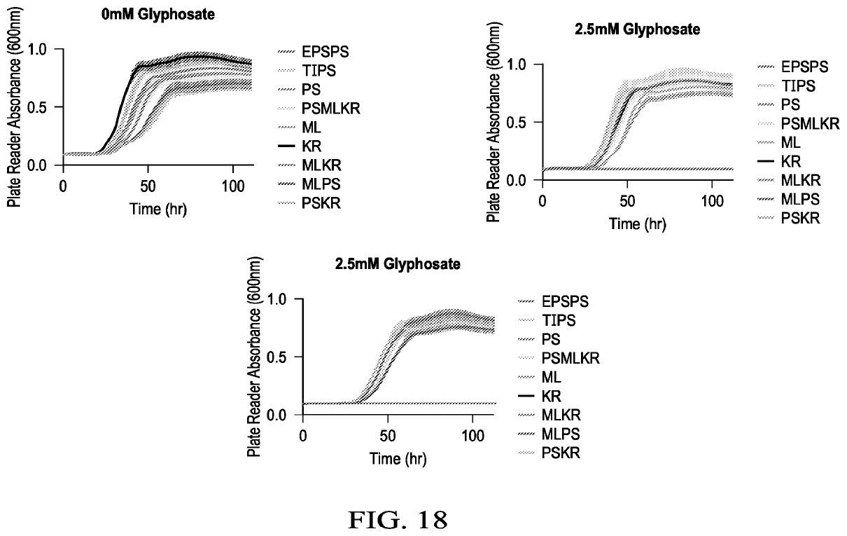

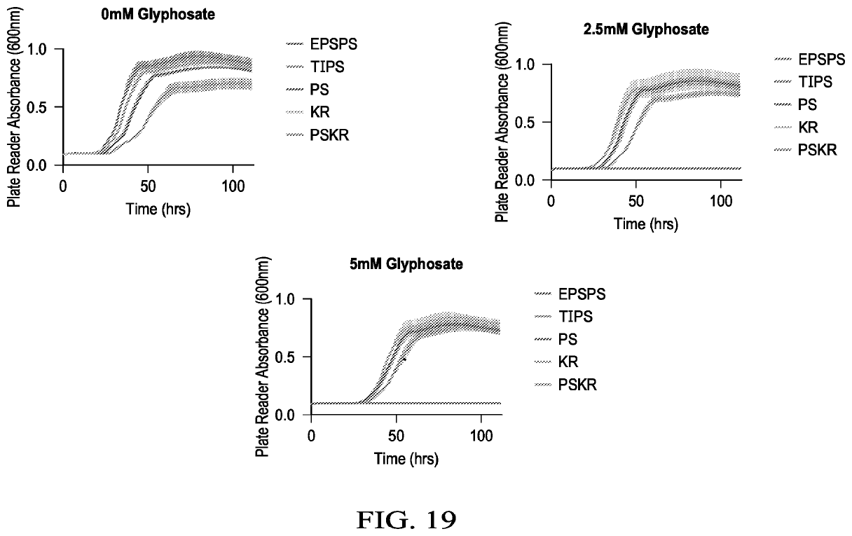

In one embodiment, the top performing mutant, TIPS-P126S-M217L-K296R and single mutant TIPS P126S were further evaluated more rigorously to determine specific growth rates in comparison to EPSPS and TIPS. TIPS P126S/M217L/K296R conferred high glyphosate tolerance (even higher than TIPS) and retained normal growth characteristics in the synthetic yeast host, similar to that of EPSPS, with only a slightly extended lag time. To determine the underlying mutations here that led to the highest improvement in growth phenotype, next were generated each combination of mutant TIPS-P126S-M217L-K296R (FIG. 18) and found that M217L did not confer any improved growth traits and may even be slightly deleterious in some conditions. Moreover, both P126S and K296R as single mutants improved growth in non-glyphosate media compared to TIPS and the additive effect of both P126S and K296R can further improve growth phenotype to nearly wild-type growth levels (FIG. 19).

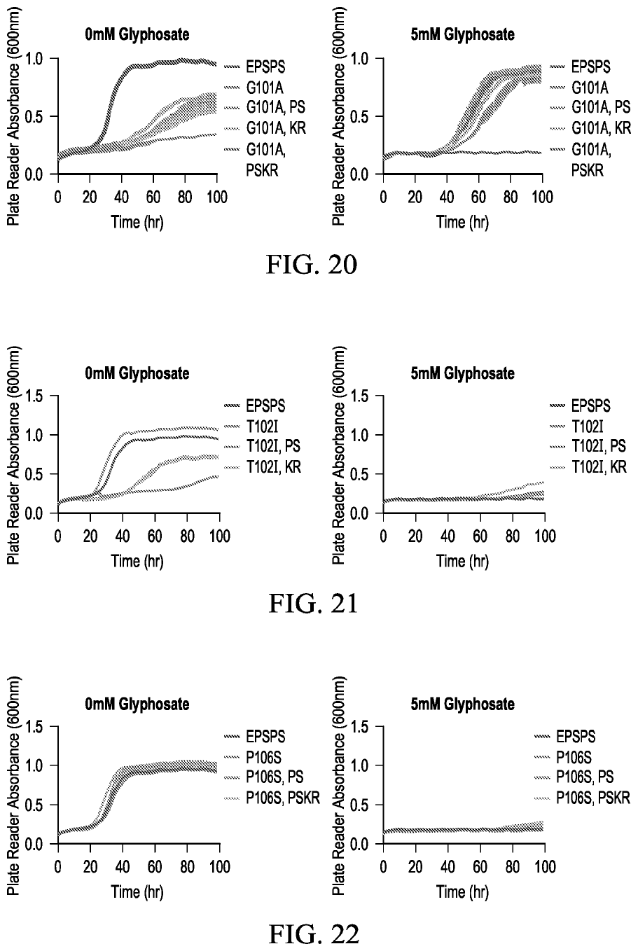

Next, the portability of mutants P126S and K296R to other glyphosate resistant mutant scaffolds including G101A (FIG. 20), T102I (FIG. 21), and P106S (FIG. 22) was determined. It was found that these mutations were capable of conferring improved growth in both 0 mM and 5 mM glyphosate for all known glyphosate resistant single mutant backgrounds. Of note, significant improvements were observed to both G101A and T102I in 0 mM, two mutants that independently have abysmally low catalytic efficiencies and strong growth defects. Furthermore, mutations P126S and K296R, in certain combinations, led to improved growth in 5 mM glyphosate for certain variants. For example, P106S with double mutant P106S-K296R was capable of observable growth in 5 mM glyphosate, whereas P106S on its own exhibited no growth in high levels of glyphosate.

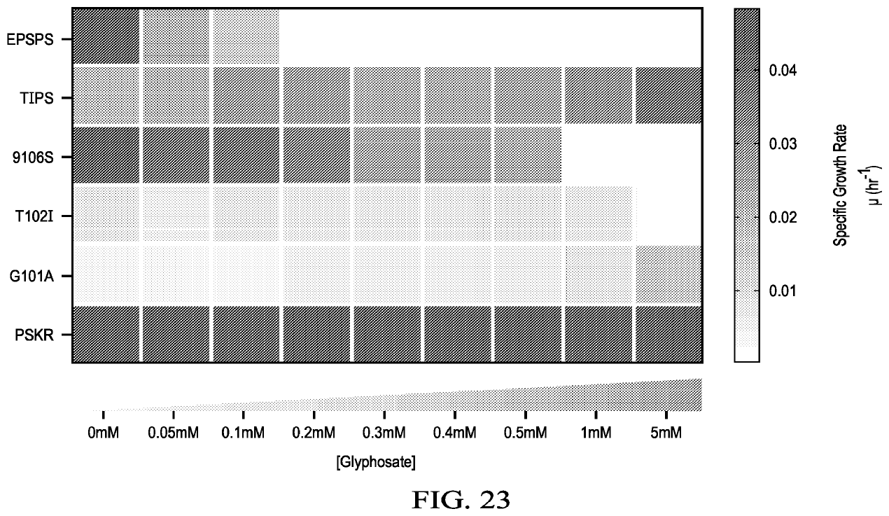

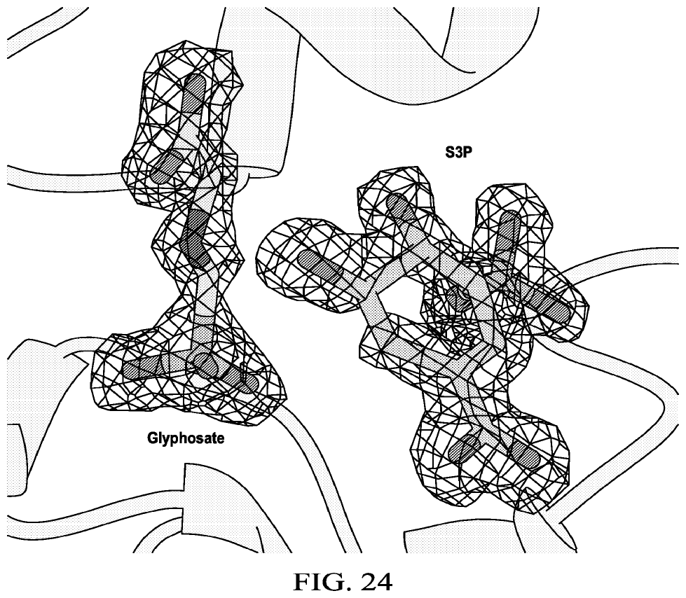

Lastly, mutant TIPS-P106S-K296R was evaluated at varying levels of glyphosate, from mild to extreme. TIPS-P106S-K296R performed extremely well in every condition of glyphosate, ranging from 0.05 mM to 5 mM, and as shown previously, performed very similarly to wild-type EPSPS in 0 mM glyphosate (FIG. 23). TIPS-P106S-K296R is thus extremely robust and confers a highly similar growth rate across any condition, further supporting its potential use in planta.

### In Vitro Characterization and Crystallization of TIPS P126S, K296R

In another embodiment, the highest performing mutant, TIPS-P106S-K296R, was characterized in vitro to further understand the kinetic and structural basis of improvement. As shown in Table 3, the enzymatic efficiency of mutant TIPS-P106S-K296R is significantly higher, roughly 2.5-fold, than the evolutionary starting point TIPS. Enzymatic activity, including inhibitory constant Kᵢ, for EPSPS and TIPS closely corroborate values determined by previous reports. Mutations P126S and K296R have beneficial effects on their own to both Kₘ and k_{cat} and are additive when combined, similar to the observations of the in vivo growth characterization. Furthermore, the high Kᵢ is maintained for each of the variants, showing similar or slightly higher values of around 800 μM compared to the starting point, TIPS. These findings confirm the phenotype endowed by the dual-trait selection pressure employed during the directed evolution campaigns. Kinetic parameters of maize EPSPS with mutations discovered through directed evolution. In Table 4, initial velocity data were measured in triplicate and fit to the Michaelis-Menten equation. Values represent mean±S.E. for 3 sets of rate measurements. Values for Kₘ were obtained by varying one substrate, with the other present at saturation (roughly 10 times its Kₘ) and therefore arc apparent Kₘ. Values shown for k_{cat} are those obtained from substrate saturation with PEP, which in no case differed significantly from those obtained with S3P.

TABLE 3

| | | Kₘ | Kₘ | k_{cat}/Kₘ | k_{cat}/Kₘ | |
|---|---|---|---|---|---|---|
| Variant | k_{cat} | (PEP) | (S3P) | (PEP) | (S3P) | Kᵢ |
| EPSPS | 2138 + 33 | 15.2 + 1.4 | 19.0 + 1.6 | 141 | 113 | 0.060 + 0.007 |
| TIPS | 152 + 9.9 | 26.2 + 3.2 | 33.2 + 4.2 | 6 | 5 | 739 + 52 |
| TIPS-P126S | 214 + 10.2 | 13.7 + 1.6 | 28.2 + 3.0 | 16 | 8 | 859 + 42 |
| TIPS-K296R | 165 + 13.3 | 24.0 + 2.5 | 16.5 + 2.3 | 7 | 10 | 841 + 61 |
| TIPS-P126S- | 224 + 14.0 | 15.5 + 0.7 | 16.8 + 2.1 | 15 | 14 | 871 + 54 |
| K296R | | | | | | |

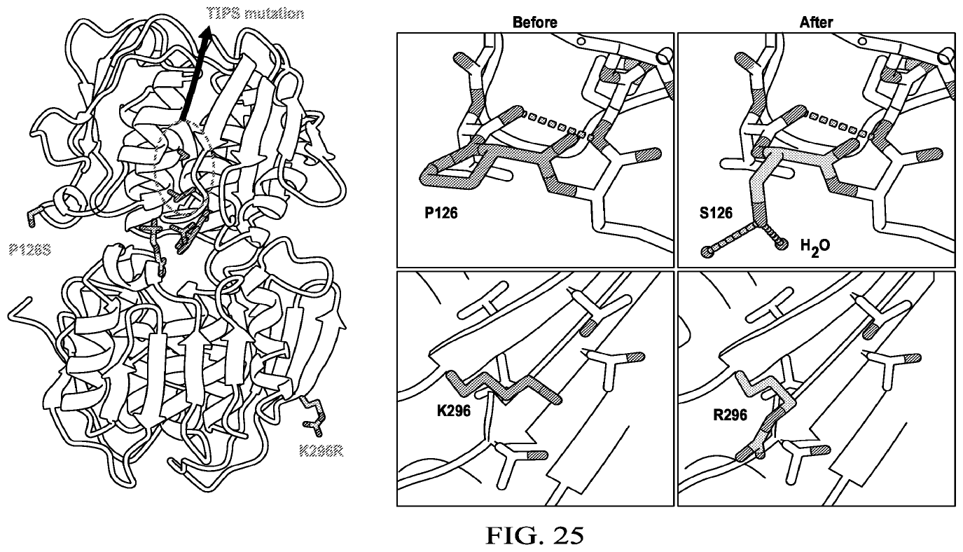

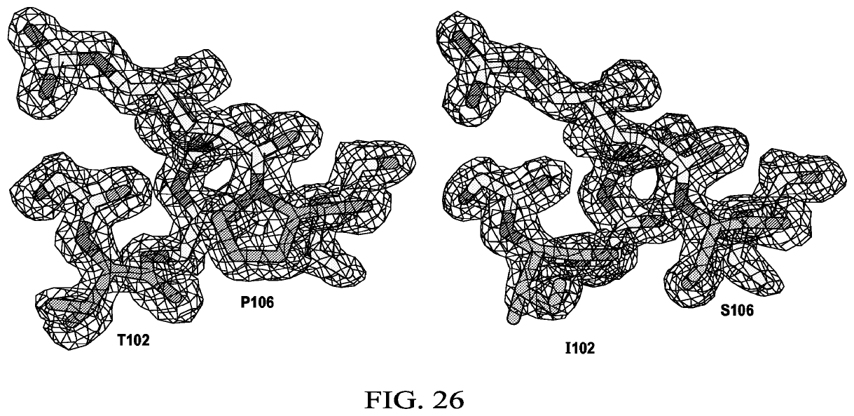

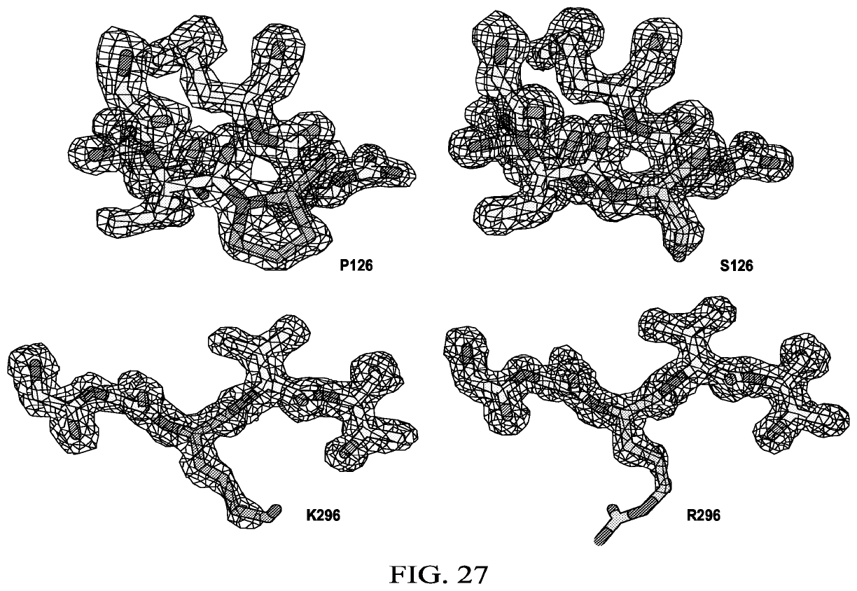

To understand how P126S and K296R mutations improve the enzyme activity, all Zea mays EPSPS variants (Wild-type, TIPS, TIPS-P126S, TIPS-K296R, TIPS-P126S-K296R) were investigated by X-ray crystallography. The crystal structure of ZmEPSPS has not yet been reported in the literature, only deduced in silico by similarity based on the recently reported structure of Arabidopsis thaliana EPSPS31. Each of the five variants was co-crystallized with shikimate-3-phosphate (S3P) and glyphosate to capture the state close to the active reaction center. The crystals diffracted, and the final structures were refined with resolutions extending to ~2 Å for all five variants (Table 3, above). With the low symmetry space group (P1) for the protein, it was only possible to scale two of the variants (TIPS-K296R and TIPS-P126S-K296R) to completeness of less than 90% due to the limitation of crystal orientation (Table 3, above). However, all ligand densities were very clear in the structures (FIG. 24). This analysis showcased that Z. mays EPSPS preserves an identical configuration to that of other reported EPSP synthases. As observed previously, the domains are highly dynamic and close upon ligand association. For each of the mutants, the locations and identities of the mutated residues were clearly distinguishable from the high-resolution structures (FIG. 25, FIG. 26 and FIG. 27), yet the overall folding of the protein was nearly identical. The structure of TIPS variant is also consistent with the previous observation that TIPS mutation endows glyphosate resistance to the enzyme by shifting the location of Gly101.

Unlike the TIPS mutation sites, P126S and K296R locate ~14 Å and ~26 Å away from the active site, thus making it impossible to directly contribute to protein-ligand interactions (FIG. 25). Nevertheless, both mutations stabilize the surface energy of protein and serve to allosterically improve the enzyme activity. For example, P126 is a nonpolar residue located at a sharp beta-turn at the protein surface of EPSPS. A small polar residue like serine replaces proline, relieving the torsion tension of such a tight turn while being further stabilized by the solvation effect (FIG. 25). On the other side of the protein surface, K296 is a positively charged residue that another positively charged residue, arginine, can replace. Although the interaction around K296 (or R296) is not well defined due to structure flexibility, substitution with arginine may provide better surface stability due to increased electrostatic interactions or hydrogen bonding (FIG. 25).

Selection systems are critical for determining the outcomes of directed evolution campaigns. In this example, a synthetic yeast host was developed to characterize and evolve novel EPSP synthase variants. By evaluating various selection pressures and the outcomes of multiple mutagenesis pools, the present synthetic yeast host can enable the selection of EPSPS variants with tunable dual traits through outgrowth in media lacking or containing glyphosate. Using these flexible selections, it is possible to isolate variants with specific phenotypes simply by altering the outgrowth conditions and varying the number of subcultures. This resulted in the isolation of variants with properties ranging from low glyphosate tolerance or low enzymatic efficiency to variants with combined high glyphosate tolerance and high enzymatic efficiency. By balancing the selection pressure through dual-trait outgrowth stages, the well-studied double mutant TIPS was evolved to grow better in a wide range of glyphosate exposure levels with only a single round of evolution. In doing so, the mutational and phenotypic landscape of evolutionary outcomes for this protein was characterized. With the most promising protein variants, changes of a few mutations can surprisingly boost enzymatic efficiency in vitro multiple fold while maintaining robust glyphosate tolerance. Given the findings here, more targeted directed evolution campaigns, such as to the loop region near residues 125-130 that closes upon binding of S3P, and additional mutagenesis rounds would likely yield fruitful outcomes with improved phenotypes. The present synthetic yeast host is useful for rapidly prototyping novel EPSPS variants that can be further studied in planta. Altogether, this example highlights the potential for dynamic selection criteria to tune proteins towards a specific dual-trait phenotype of interest and for yeast models as a general synthetic tool to study herbicide mechanisms.

### Example 2: Characterization of Selected Variants in Zea mays Plants

The EPSPS variants were characterized in a plant system to determine if any of the observed improvements showed biological improvements in planta. Maize was used as a model system to test the functionality of the EPSPS variants because of detailed previous characterization of glyphosate tolerance in maize and a suitable transgenic plant generation protocol that relies on glyphosate. Plant transformation vectors were generated in which the native maize EPSPS sequence was used, which includes the native gene promoter elements, the native introns and exons and the native 3′ untranslated region and introduced mutations that corresponded to each of the selected amino acid changes. The native sequence was used to mimic as closely as possible how the selected EPSPS variants would perform if the variants were introduced directly in the genome without the help of strong expression elements. The following variants were selected for testing in a transgenic maize system: TIPS (as a control); TIPS-P126S-M217L-K296R; TIPS-P126S-K296R; T102I-P126S-K296R (where only the T102I change is present); P106S-P126S-K296R (where only the P106S change is present); and P126S-K296R (where neither of the TIPS changes are present).

Transgenic maize plants that expressed each of the six EPSPS variants were generated to determine if the variants conferred glyphosate tolerance to plants. The full DNA sequence encoding TIPS EPSPS (SEQ ID NO:1) was cloned from maize genomic DNA. In this sequence, the promoter and 5′ UTR are nucleotides 1:2556; the chloroplast transit peptide sequence is nucleotides 2557:2742; EXON 1 is nucleotides 2743:2856; INTRON 1 is nucleotides 2857:3384; EXON 2 is nucleotides 3385:3626; INTRON 2 is nucleotides 3627:3725; EXON 3 is nucleotides 3726:3879; INTRON 3 is nucleotides 3880:4152; EXON 4 is nucleotides 4153:4367; INTRON 4 is nucleotides 4368:4877; EXON 5 is nucleotides 4878:4995; INTRON 5 is nucleotides 4996:5155; EXON 6 is nucleotides 5156:5366; INTRON 6 is nucleotides 5367:5446; EXON 7 is nucleotides 5447:5508; INTRON 7 is nucleotides 5509:5617; EXON 8 is nucleotides 5618:5836; and the 3′ UTR is nucleotides 5837:6368. Mutations were then introduced into the EPSPS coding sequence in order to produce each of the six EPSPS variants to be tested. These mutated full genomic DNA sequences were then each cloned as a single expression cassette into plant transformation vectors.

Agrobacterium tumefaciens-mediated plant transformation was performed using methods known in the art. Glyphosate selection was used to select for transgenic events containing the transgene of interest and further screened for only those EPSPS variants that provided effective glyphosate tolerance in a plant cell. While all six EPSPS variants began the transformation process with the same amount of starting maize cellular material, there were differences in the number of small plantlets recovered that were capable of being transplanted to greenhouse soil media (Table 4). Transgenic events were further analyzed to determine transgene copy number. Healthy events with single copy of the transgene of interest were grown in the greenhouse soil media. Only three of the six EPSPS variants had more than one plant surviving. Up to twenty-five of these developing R0 plants were transplanted to larger pots and allowed to grow to maturity for seed collection in the greenhouse.

Two of the six EPSPS variants had developing plants greater than twenty-five. The extra developing R0 plants from these two variants were utilized in a glyphosate spray test to determine the tolerance provided by the EPSPS variants to a typical post glyphosate application to young plants. The R0 plants were sprayed with glyphosate applied postemergence (POST) at 0.75 lb ae (acid equivalent)/acre (0.84 kg ae/ha) at the V3-V4 stage. Treated plants were evaluated for injury seven days after glyphosate application.

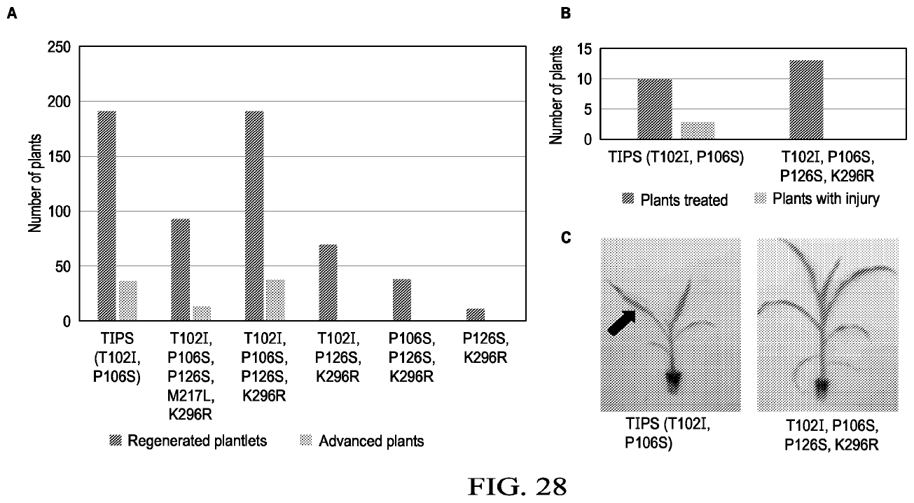

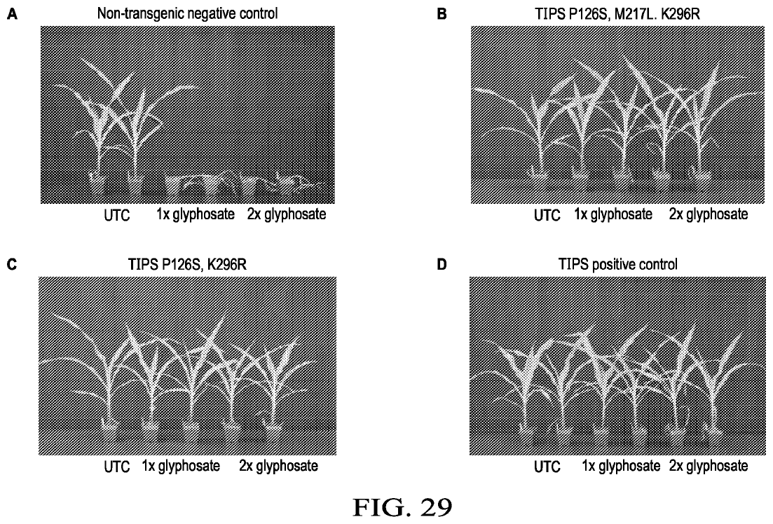

The results showed that two of the experimental EPSPS variants, TIPS-P126S-M217L-K296R and TIPS-P126S-K296R, were capable of providing glyphosate tolerance to enable successful transformation and growth of healthy plants to produce seeds. One of these experimental EPSPS variants, TIPS-P126S-K296R, produced young plants that showed better glyphosate tolerance than the TIPS control with 0% of the sprayed plants showing injury while 30% of the TIPS control plants showed injury (Table 4). The results are shown in FIG. 28.

TABLE 4

| | | | | Percent of |
|---|---|---|---|---|
| | Number | Number of | Number of | sprayed plants |
| | of | young plants | plants | showing |
| | plantlets | passing quality | sprayed with | glyphosate |
| EPSPS Variant | generated | control checks | glyphosate | injury |
| TIPS | 192 | 35 | 10 | 30% |
| TIPS-P126S-M217L-K296R | 92 | 13 | 0 | NA |
| TIPS-P126S-K296R | 190 | 38 | 13 | 0% |
| T102I-P126S-K296R | 69 | 1 | 0 | NA |
| P106S-P126S-K296R | 39 | 0 | 0 | NA |
| P126S-K296R | 11 | 1 | 0 | NA |

### Example 3: In Planta Testing of Selected EPSPS Variants in Maize

To further evaluate the performance of EPSPS variants for glyphosate tolerance, transgenic maize plants comprising a single copy of the transgene encoding the EPSPS variants TIPS-P126S-M217L-K296R or TIPS-P126S-K296R were grown to maturity. Homozygous transgenic plants were crossed with an inbred line to produce hybrid seeds.

Plants from the hybrid seeds (hemizygous for the transgenes) of two independent transformation events for each of the two variants were grown in the greenhouse for glyphosate spray test. Roundup® PowerMax3, a commercial formulation of glyphosate, was applied at the third leaf stage at two rates: 1120 gram/hectare (1× the field rate) and 2240 gram/hectare (2× the field rate). The controls included an untreated untransformed control without glyphosate treatment, an untransformed control sprayed with glyphosate, and a TIPS positive control both sprayed with glyphosate. Glyphosate herbicide injury (plant stunting, chlorosis, necrosis, malformation, and death) was assessed by visual inspection of the plants 18 days after herbicide treatment. Herbicide injury rate was presented on a scale of 0 to 100 with “0” being no visible crop injury and “100” being complete crop injury or death.

The results of the glyphosate spray test are summarized in Table 5. Both TIPS-P126S-M217L-K296R and TIPS-P126S-K296R variants provided excellent tolerance to an equivalent 1× field dose of glyphosate. Furthermore, variant TIPS-P126S-M217L-K296R provided full tolerance to the 2× field rate indicating robust tolerance. Both TIPS-P126S-K296R and the TIPS control plants showed minor injuries at the 2× field rate. This testing confirms that both TIPS-P126S-M217L-K296R and TIPS-P126S-K296R EPSPS variants can provide robust tolerance in greenhouse testing designed to mimic field conditions.

TABLE 5
Performance of EPSPS Variants Following Glyphosate Application

| | Average Injury | | | |
|---|---|---|---|---|
| | At 1120 | Std. | At 2240 | Std. |
| EPSPS Variant | g/ha | Dev. | g/ha | Dev. |
| TIPS | 0 | 0 | 1.25 | 5 |
| TIPS-P126S-M217L-K296R | 0 | 0 | 0 | 0 |
| TIPS-P126S-K296R | 0 | 0 | 17 | 15.85 |
| Untransformed Control | 100 | 0 | 100 | 0 |

The Examples illustrate certain embodiments of the present disclosure. It should be appreciated by those of skilled in the art that many modifications can be made in the specific examples which are disclosed and still obtain a similar result. Certain agents which are both chemically and physiologically related may be substituted for the agents described herein while achieving the same or similar results. All such substitutions and modifications apparent to those skilled in the art are deemed to be within the scope of the invention.

1. A recombinant DNA molecule encoding a glyphosate-tolerant 5-enol-pyruvyl-shikimate-3-phosphate synthase (EPSPS), wherein the EPSPS comprises at least a first amino acid substitution combination selected from the group consisting of: T102I-P106S-P126S-K296R, T102I-P106S-P126S, T102I-P106S-K296R, T102I-P106S-P126S-M217L-K296R, T102I-P106S-V66L-M104L, T102I-P106S-T108S, T102I-P106S-F150V-H318Y, T102I-P106S-P126L, T102I-P106S-G116V, T102I-P106S-1177V, and P106S-A109V-1163K, wherein the position of the amino acid substitution is relative to the position of the amino acid sequence provided as SEQ ID NO: 6.

2. The recombinant DNA molecule of claim 1, wherein the EPSPS is a maize EPSPS.

3. The recombinant DNA molecule of claim 1, wherein the EPSPS confers increased tolerance to glyphosate or increased enzymatic efficiency as compared to an EPSPS lacking said combination.

4. The recombinant DNA molecule of claim 1, wherein the EPSPS comprises at least two of said substitution combinations.

5. A glyphosate-tolerant EPSPS comprising at least a first amino acid substitution combination selected from the group consisting of: T102I-P106S-P126S-K296R, T102I-P106S-P126S, T102I-P106S-K296R, T102I-P106S-P126S-M217L-K296R, T102I-P106S-V66L-M104L, T102I-P106S-T108S, T102I-P106S-F150V-H318Y, T102I-P106S-P126L, T102I-P106S-G116V, T102I-P106S-1177V, and P106S-A109V-1163K, wherein the position of the amino acid substitution is relative to the position of the amino acid sequence provided as SEQ ID NO: 6.

6. The glyphosate-tolerant EPSPS of claim 5, wherein the EPSPS confers increased tolerance to glyphosate or increased enzymatic efficiency as compared to an EPSPS lacking said combination, or as compared to an EPSPS containing only T102I-P106S.

7. The glyphosate-tolerant EPSPS of claim 5, wherein the EPSPS comprises at least two of said substitution combinations.

8. A plant, seed, cell, plant part, or commodity product comprising the recombinant DNA molecule of claim 1.

9. The plant of claim 8, wherein the plant exhibits increased glyphosate tolerance when compared to a plant lacking said combination.

10. The plant of claim 9, wherein the plant is a corn, soy, cotton, canola, wheat, rice, alfalfa, sugar beet, oilseed rape or sugar cane plant.

11. A transgenic maize plant comprising a recombinant DNA molecule encoding a glyphosate-tolerant 5-enol-pyruvyl-shikimate-3-phosphate synthase (EPSPS), wherein the EPSPS comprises at least a first amino acid substitution combination selected from the group consisting of: T102I-P106S-P126S-K296R, T102I-P106S-P126S, T102I-P106S-K296R, T102I-P106S-P126S-M217L-K296R, T102I-P106S-V66L-M104L, T102I-P106S-T108S, T102I-P106S-F150V-H318Y, T102I-P106S-P126L, T102I-P106S-G116V, T102I-P106S-1177V, and P106S-A109V-I163K, wherein the position of the amino acid substitution is relative to the position of the amino acid sequence provided as SEQ ID NO:6, and wherein said EPSPS confers glyphosate tolerance to said transgenic maize plant.

12. The transgenic maize plant of claim 11, wherein said glyphosate tolerance is greater than the glyphosate tolerance conferred by said EPSPS that comprises only a T102I-P106S substitution.

13. A seed, cell or plant part of the plant of claim 11, wherein the seed, cell or plant part comprises said recombinant DNA molecule.

14. A method for conferring glyphosate tolerance to a plant comprising expressing in the plant the glyphosate-tolerant EPSPS of claim 5.

15. The method of claim 14, comprising introducing the recombinant DNA molecule of claim 1 into the genome of said plant or a progenitor thereof.

16. A method for producing a glyphosate-tolerant EPSPS comprising introducing into a nucleic acid molecule encoding a plant EPSPS at least a first amino acid substitution combination selected from the group consisting of: T102I-P106S-P126S-K296R, T102I-P106S-P126S, T102I-P106S-K296R, T102I-P106S-P126S-M217L-K296R, T102I-P106S-V66L-M104L, T102I-P106S-T108S, T102I-P106S-F150V-H318Y, T102I-P106S-P126L, T102I-P106S-G116V, T102I-P106S-1177V, and P106S-A109V-1163K, wherein the position of the amino acid substitution is relative to the position of the amino acid sequence provided as SEQ ID NO:6.

17. The method of claim 16, wherein said introducing is carried out in vitro.

18. The method of claim 16, wherein said introducing is carried out in planta.

19. The method of claim 16, wherein said introducing comprises use of at least a first site-specific endonuclease.

20. The method of claim 16, wherein the method comprises introducing at least two of said substitution combinations into said EPSPS.

21. A method for controlling weeds in a plant growth area, comprising contacting a plant growth area comprising the plant or seed of claim 8 with glyphosate, wherein the plant or seed is tolerant to glyphosate, and wherein weeds are controlled in the plant growth area.

22. A yeast cell comprising a knockout of native aro1 function, and further comprising a recombinant DNA molecule comprising a heterologous promoter operably linked to an aro1 coding sequence comprising a D731A mutation, wherein said yeast cell is not able to grow on media without aromatic amino acids.

23. The yeast cell of claim 22, further comprising a heterologous nucleic acid molecule encoding an EPSPS, wherein said yeast cell is able to grow on media without aromatic amino acids.

24. The yeast cell of claim 22, wherein the EPSPS is a glyphosate-tolerant maize EPSPS.

25. The yeast cell of claim 22, wherein the yeast cell is a Saccharomyces cerevisiae cell.

26. A method for identifying a glyphosate tolerant EPSPS having improved growth, improved glyphosate tolerance or improved enzymatic efficiency when compared to wild-type EPSPS, comprising the steps of:
a) obtaining a yeast cell according to claim 22; and
b) identifying the yeast cell as capable of growing in the presence of glyphosate and therefore comprising a glyphosate tolerant EPSPS having improved growth, improved glyphosate tolerance or improved enzymatic efficiency when compared to wild-type EPSPS.

27. The method of claim 26, wherein the method comprises obtaining a population of yeast cells collectively comprising a plurality of heterologous nucleic acid molecules encoding mutant EPSPS proteins and identifying at least one of the yeast cells as having improved growth, improved glyphosate tolerance or improved enzymatic efficiency.

28. The method of claim 26, further comprising cloning the nucleic acid molecule encoding said glyphosate tolerant EPSPS from said yeast cell or a progeny thereof.

29. A method of identifying a plant, seed, cell, or plant part as comprising glyphosate tolerance, the method comprising applying glyphosate to the plant, seed, cell, or plant part that comprises the recombinant DNA molecule of claim 1.

30. The method of claim 29, further comprising applying said glyphosate to a population of plants, seeds, cells, or plant parts.

31. The method of claim 30, comprising applying said glyphosate to a culture of cells.
